(19) United States
(12) Patent Application Publication
(10) Pub. No.: US 2023/0293406 A1
(43) Pub. Date: Sep. 21, 2023

(54) Compositions Comprising (Bio)-Alkanediols and Cooling Agents

(71) Applicant: Symrise AG, Holzminden (DE)

(72) Inventors: Christin Koch, Detmold (DE); Sabine Lange, Holzminden (DE); Nikolas Bugdahn, Holzminden (DE); Ricarda Kraeling, Holzminden (DE); Steffen Nordzieke, Göttingen (DE)

(21) Appl. No.: 18/009,291

(22) PCT Filed: Dec. 9, 2021
(86) PCT No.: PCT/EP2021/084969

§ 371 Date: Dec. 8, 2022

(30) Foreign Application Priority Data
Dec. 9, 2020 (WO) PCT/EP2020/085168

(51) Int. Cl. A61K 8/34; A61K 47/44; A61K 47/10; A61K 8/92; A61Q 19/00; A61Q 15/00; A61K 8/37; A61Q 17/00

(52) U.S. Cl. CPC A61K 8/345; A61K 47/44; A61K 47/10; A61K 8/922; A61Q 19/00; A61Q 15/00; A61K 8/34; A61K 8/37; A61Q 17/005; A61K 2800/10

(57) ABSTRACT

The present invention relates to a homecare product composition or a cosmetic or pharmaceutical composition comprising or consisting of an effective amount of 1,2-nonanediol or 2,3-nonanediol or a mixture comprising 1,2-nonanediol and 2,3-nonanediol or of a specific alkanediol having a carbon chain of 5 to 14 carbon atoms or a mixture of two or more different specific alkanediols having a carbon chain of 5 to 14 carbon atoms, in combination with an effective amount of at least one cooling agent. Additionally, the present invention relates to the use of 1,2-nonanediol or 2,3-nonanediol or a mixture comprising 1,2-nonanediol and 2,3-nonanediol or the use of a specific alkanediol having a carbon chain of 5 to 14 carbon atoms or a mixture of two or more different specific alkanediols having a carbon chain of 5 to 14 carbon atoms for malodour reduction or for inhibiting malodour development or for body odour control or for masking insect alluring odours. Furthermore, the present invention relates to a cosmetic product comprising a cosmetic composition of the present invention. Finally, the present invention relates to the use of 1,2-nonanediol and/or 2,3-nonanediol or of a specific alkanediol having a carbon chain of 5 to 14 carbon atoms or a mixture of two or more different specific alkanediols having a carbon chain of 5 to 14 carbon atoms for preparing a cosmetic product of the present invention or a homecare product.

### TECHNICAL FIELD

The present invention relates to a homecare product composition or cosmetic or pharmaceutical composition comprising or consisting of an effective amount of 1,2-nonanediol and/or 2,3-nonanediol or of a specific alkanediol or a mixture of two or more different specific alkanediols, in combination with an effective amount of at least one cooling agent. Additionally, the present invention relates to the use of 1,2-nonanediol and/or 2,3-nonanediol or of a specific alkanediol or a mixture of two or more different specific alkanediols for malodour reduction or for inhibiting malodour development or for body odour control or for masking insect alluring odours. Furthermore, the present invention relates to a cosmetic product comprising a cosmetic composition of the present invention. Finally, the present invention relates to the use of 1,2-nonanediol and/or 2,3-nonanediol or of a specific alkanediol or a mixture of two or more different specific alkanediols for producing a cosmetic product of the present invention or a homecare product.

### BACKGROUND ART

The origin of malodour or body odour of humans or other mammalians usually lies in the sweat glands located in the skin. There are two different types of sweat glands the eccrine sweat glands and the apocrine sweat glands. In humans the eccrine sweat glands are distributed all over the body and they produce a water-based secretion for cooling the body. The locations of the apocrine sweat glands are rather limited and in humans they are located e.g. in the armpits. The apocrine sweat glands produce a substance that is thicker than eccrine sweat and provides nutrients for bacteria on the skin. In addition, the apocrine sweat contains other compounds e.g. pheromone-like compounds.

It is known that microbes living on the human skin produce a variety of different compounds like formic acid, butyric acid, 3-hydroxy-3-methylhexanoic acid and 3-methyl-3-sulfanylhexane-1-ol from the sweat produced by the apocrine sweat glands. Several compounds produced by the microbes are responsible for an unpleasant odour e.g. butyric acid has a very intense and unpleasant odour.

In this context, important microorganisms living on the skin or scalp of humans are microorganisms from the genus Staphylococcus, Corynebactrerium, Anaerococcus, Finegoldia, Moraxella, Porphyromonas, Fusobacterium, Aspergillus, Candida, Malassezia, Escherichia, Peptoniphilus; Streptococcus, Lactobacillus; Gardnerella, Fannyhessea, Epidermophyton, Trichophyton, Fusobacterium, Cutibacterium, in particular the microorganisms Staphylococcus epidermidis; Staphylococcus hominis; Corynebacterium xerosis; Corynebacterium jeikeium; Anaerococcus octavius; Finegoldia magna; Moraxella osloensis; Moraxella atlantae; Porphyromonas gingivalis; Aspergillus brasiliensis; Candida albicans; Escherichia coli; Staphylococcus aureus; Peptoniphilus lacrimalis; Streptococcus agalactiae; Lactobacillus acidophilus; Gardnerella vaginalis; Fannyhessea vaginae (Atopobium vaginae); Epidermophyton floccosum; Trichophyton rubrum; Streptococcus mutans; Fusobacterium nucleatum. In the past, the contributions of Anaerococcus octavius and Corynebacterium jeikeium to body odour development have been underestimated. Recently, it has been discovered that the microorganisms Anaerococcus octavius and Corynebacterium jeikeium are of special relevance for the production of compounds having an unpleasant smell from human sweat.

In addition, the pheromone-like compounds like the steroids androstenone and androsterone sulfate which are directly produced by the apocrine sweat glands also are responsible for an unpleasant malodour or body odour.

The compounds produced by microbes from human or animal sweat, and the compounds directly produced by the apocrine sweat glands also lead to a malodour smell of old age.

To efficiently reduce malodour or inhibit malodour development or control body odour a compound or composition needs to reduce the formation of sweat and/or the microbial activity on the skin. Additionally, the compound or composition must cover the unpleasant smell of different compounds produced by microbes or directly produced by the sweat glands.

In this context, it is to be noted that the substances used in cosmetics and pharmaceuticals as well as in homecare products must fulfil certain requirements. The compounds must be toxicologically acceptable, readily tolerated by the skin, stable in conventional cosmetic and/or pharmaceutical formulations or homecare product formulations, inexpensive to prepare, easy to formulate and the compounds need to be active at different pH-values. Furthermore, the substance must not have an unpleasant smell. In the search for new substances which are useful in cosmetic and pharmaceutical compositions there is no predictable connection between the chemical structure and other physico-chemical parameters relevant to the field of cosmetics and pharmaceuticals, i.e. the toxicological acceptability, the skin tolerability, the stability, solubility and formulation properties and the smell of a substance. Moreover, homecare products such as laundry detergents (in powdered or liquid form), fabric conditioner, dishwashing detergents, floor and surface cleaners, glass cleaners, air freshers and the like should be able to efficiently reduce the malodour or unpleasant smell such as from human sweats or from animal sweats on home furnishings and textiles such as pillows, sofa covers etc., clothes, carpets and the like.

EP 2774481 B1 discloses an antimicrobial mixture comprising 4-hydroxyacetophenone and at least one second antimicrobial agent selected from the group consisting of 1,3-propanediol, methyl propanediol, 1,2-pentanediol, 1,2-hexanediol, 1,2-octanediol, ethylhexylglycerin, hexoxy-propan-1,2-diol, heptoxy-propan-1,2-diol, octoxy-propan-1,2-diol, 3-benzyloxy-propan-1,2-diol, 3-phenyl-ethyloxy-propan-1,2-diol, 3-phenylpropyloxy-propan-1,2-diol, 3-methylbenzyloxy-propan-1,2-diol, 3-phenyl propanol, 2-phenoxyethanol. The antimicrobial mixtures have a synergistic antimicrobial effect and can be used for combating underarm and foot odour or body odour in general.

However, the homecare, cosmetic and pharmaceutical industry is permanently searching for new ingredients and compositions which can further improve the usefulness of homecare products as well as cosmetic and pharmaceutical compositions in malodour reduction or inhibiting malodour development or body odour control, respectively.

### SUMMARY OF THE INVENTION

It is a problem of the present invention to provide compounds and cosmetic or pharmaceutical compositions or homecare product compositions which can be efficiently used in malodour reduction or inhibiting malodour development or body odour control or masking insect alluring odours.

In order to accomplish the above problem, the present invention provides a cosmetic or pharmaceutical composition or a homecare product composition comprising or consisting of
(a1) 1,2-nonanediol;
(b1) at least one cooling agent;
(c1) optionally at least one antimicrobial compound; and
(d1) optionally at least one active substance for a cosmetic or pharmaceutical composition or a homecare product composition and/or additive;
or
(a2) 2,3-nonanediol;
(b2) at least one cooling agent;
(c2) optionally at least one antimicrobial compound; and
(d2) optionally at least one active substance for a cosmetic or pharmaceutical composition or a homecare product composition and/or additive;
or
(a3) a mixture of 1,2-nonanediol and 2,3-nonanediol;
(b3) at least one cooling agent;
(c3) optionally at least one antimicrobial compound; and
(d3) optionally at least one active substance for a cosmetic or pharmaceutical composition or a homecare product composition and/or additive.

Furthermore, the present invention provides a cosmetic or pharmaceutical composition or a homecare product composition, comprising or consisting of
(a) at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol;
(b) at least one cooling agent;
(c) optionally at least one antimicrobial compound; and
(d) optionally at least one active substance for a cosmetic or pharmaceutical composition or a homecare product composition and/or additive.

Moreover, the present invention provides for the use of 1,2-nonanediol and/or 2,3-nonanediol, or at least one linear alkanediol or a mixture of at least one first linear alkanediol with one or more second linear alkanediols as defined above for malodour reduction or for inhibiting malodour development or for body odour control or for masking insect alluring odours.

In addition, the present invention provides a cosmetic product comprising a cosmetic composition according to the present invention, wherein the product is a deodorant and/or antiperspirant, an aerosol-based deo spray, deo pump spray, deo stick, deo roll on, deo cream, deo wipes, deo crystals, pickering emulsions, hydrodisperion gels, skin balms, shampoo, shower gel, foam bath, micellar water, facial cleansing solutions, cleansing wipes, intimate spray, intimate cream, intimate wash lotion, intimate wipes, aerosol-based foot spray, foot pump spray, foot bath, foot balm, soap, liquid washing, shower and bath preparation, bath product (capsule, oil, tablet, salt, bath salt, soap, etc.), effervescent preparation, mouth wash concentrate, mouth wash ready to use, repellent, anti-insect application, in particular insect repellent lotion, spray, solution or cream, mosquito repellent, human-smell-masking application, perfume composition and tooth paste.

Finally, the present invention provides for the use of 1,2-nonanediol and/or 2,3-nonanediol, or at least one linear alkanediol or a mixture of at least one first linear alkanediol with one or more second linear alkanediols as defined above for producing a cosmetic product according to the present invention or a homecare product.

The present invention is based on the recognition that the cosmetic or pharmaceutical compositions or the homecare product composition according to the present invention comprising 1,2-nonanediol and/or 2,3-nonanediol, or at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different from the first linear alkanediol, in combination with at least one cooling agent provide an excellent cooling effect, an excellent antimicrobial effect and an excellent malodour coverage effect. Therefore, the utilisation of the cosmetic or pharmaceutical compositions or the homecare product composition according to the present invention allow for a reduced production of sweat by the sweat glands due to the excellent cooling effect. Moreover, the production of compounds having an unpleasant odour from sweat by microbes on the skin is inhibited due to the excellent antimicrobial effect. Additionally, the malodour of compounds contained in sweat is efficiently reduced due to the coverage of unpleasant odours by the cosmetic or pharmaceutical composition or the corresponding homecare product composition (malodour coverage). Moreover, insect alluring odours such as the odour of 1-octen-3-ol are efficiently covered. Consequently, the homecare product compositions as well as the cosmetic or pharmaceutical compositions according to the present invention are highly efficient in malodour reduction, in inhibiting malodour development, in body odour control and in masking insect alluring odours, respectively. Finally, the cosmetic or pharmaceutical compositions and the homecare product compositions according to the present invention are toxicologically acceptable, readily tolerated by the skin, stable, inexpensive to prepare, easy to formulate, active at different pH-values and do not possess an unpleasant smell.

### DESCRIPTION OF FIGURES

FIGS. 1a and 1b are diagrams showing the odour modification results for C7 (heptanediol) and C8 (octanediol) alkanediols.

FIG. 2 is a diagram showing the 1-octen-3-ol odour modification results for C7 (heptanediol) and C8 (octanediol) alkanediols.

FIGS. 3a and 3b are diagrams showing the masking effect according to Example 7 (Samples A, B, C, D).

### DETAILED DESCRIPTION OF THE INVENTION

The present invention is specified in the appended claims. The invention itself, and its preferred variants, other objects and advantages, are however also apparent from the following detailed description in conjunction with the accompanying examples.

In a first aspect, the present invention relates to a homecare product composition or a cosmetic or pharmaceutical composition comprising or consisting of:
(a1) 1,2-nonanediol;
(b1) at least one cooling agent;
(c1) optionally at least one antimicrobial compound; and
(d1) optionally at least one active substance for a cosmetic or pharmaceutical composition or a homecare product composition and/or additive.

Alternatively, the present invention relates in a first aspect to a homecare product composition or a cosmetic or pharmaceutical composition comprising or consisting of:
(a2) 2,3-nonanediol;
(b2) at least one cooling agent;
(c2) optionally at least one antimicrobial compound; and
(d2) optionally at least one active substance for a cosmetic or pharmaceutical composition or a homecare product composition and/or additive.

In a further alternative, the present invention relates in a first aspect to a homecare product composition or a cosmetic or pharmaceutical composition comprising or consisting of:
(a3) a mixture of 1,2-nonanediol and 2,3-nonanediol;
(b3) at least one cooling agent;
(c3) optionally at least one antimicrobial compound; and
(d3) optionally at least one active substance for a cosmetic or pharmaceutical composition or a homecare product composition and/or additive.

In a preferred variant of the first aspect of the present invention, the component (b) and/or component (c) is different from component (a).

Furthermore, in a second aspect the present invention relates to a homecare product composition or a cosmetic or pharmaceutical composition, comprising or consisting of:
(a) at least one linear alkanediol having a carbon chain of 5 to 14 carbon atoms or a mixture comprising at least one first linear alkanediol having a carbon chain of 5 to 14 carbon atoms and one or more second linear alkanediol(s) having a carbon chain of 5 to 14 carbon atoms which is different to the first linear alkanediol;
(b) at least one cooling agent;
(c) optionally at least one antimicrobial compound; and
(d) optionally at least active substance for a cosmetic or pharmaceutical composition or a homecare product composition and/or additive.

In a preferred variant of the second aspect of the present invention, the component (b) and/or component (c) is different from component (a).

In a more preferred variant of the second aspect, the present invention relates to a composition, comprising or consisting of:
(a′) a mixture comprising at least one first linear alkanediol having a carbon chain of 5 to 14 carbon atoms and one or more second linear alkanediol(s) having a carbon chain of 5 to 14 carbon atoms, wherein the number of the carbon atoms of the first and the second alkanediol is either same or different;
(b′) at least one cooling agent; and
(c′) optionally at least one antimicrobial compound; and
(d′) optionally at least active substance for a cosmetic or pharmaceutical composition or a homecare product composition and/or additive.

The term “comprising” means that the named components in the composition are essential, but other components may be added and is still embraced by the present invention. The term “consisting of” as used according to the present invention means that the total amount of components (a) to (d) adds up to 100% by weight, based on the total weight of the composition, and signifies that the subject matter is closed-ended and can only include the limitations that are expressly recited. Whenever reference is made to “comprising” it is intended to cover both meanings as alternatives, that is the meaning can be either “comprising” or “consisting of”, unless the context dictates otherwise.

The term “at least one cooling agent” means that the homecare product composition or cosmetic or pharmaceutical composition according to the present invention can comprise either one cooling agent or a mixture of two, three, four, five, six or even more different cooling agents.

The term “at least one linear alkanediol” means that the homecare product composition or cosmetic or pharmaceutical composition according to the present invention can comprise either one linear alkanediol or a mixture of two, three, four, five, six or even more different linear alkanediols having a carbon chain of 5 to 14 carbon atoms.

The term “at least one first linear alkanediol” means that the homecare product composition or cosmetic or pharmaceutical composition according to the present invention can comprise either one first linear alkanediol or a mixture of two, three, four, five, six or even more different first linear alkanediols having a carbon chain of 5 to 14 carbon atoms.

The term “one or more second linear alkanediol(s)” means that the homecare product composition or cosmetic or pharmaceutical composition according to the present invention can comprise either one second linear alkanediol or a mixture of two, three, four, five, six or even more different second linear alkanediols having a carbon chain of 5 to 14 carbon atoms.

The term “at least one antimicrobial compound” means that the homecare product composition or cosmetic or pharmaceutical composition according to the present invention can comprise either one antimicrobial compound or a mixture of two, three, four, five, six or even more different antimicrobial compounds.

The term “at least one active substance for a cosmetic or pharmaceutical composition or a homecare product composition and/or additive” means that the homecare product composition or cosmetic or pharmaceutical composition according to the present invention can comprise either active substance for a cosmetic or pharmaceutical composition or a homecare product composition and/or additive or a mixture of two, three, four, five, six or even more different active substances for a cosmetic or pharmaceutical composition or a homecare product composition and/or additives.

The term “synergistic effect” is an effect achieved when two different chemical substances or biological structures interact resulting in accumulative overall effect that is greater than the sum of individual effects of any of them. In the present invention, the synergistic effect relates to a synergistic malodour reduction efficiency.

The malodour reducing or covering effect in the context of the present invention means that the offensive or to most people unpleasant olfactory perception which is usually caused by particularly odorous substances, is perceivably reduced, covered or even inhibited. Thereby, the reduction is not merely based on masking with strong fragrance.

Antimicrobial effect in the context of the present invention means that the microorganisms' growth is reduced or even inhibited, so that the number of living microorganisms is reduced.

Alkanediols are glycols, i.e. any of a class of organic compounds belonging to the alcohol family; in the molecule of a glycol, two hydroxyl (—OH) groups are attached to different carbon atoms of a carbon chain, i.e. they are alcohols (divalent alcohols).

Component (a)

The component (a) in the composition according to the first aspect of the present invention is either 1,2-nonanediol (a1) or 2,3-nonanediol (a2), or a mixture comprising both nonanediols, i.e. 1,2-nonanediol plus 2,3-nonanediol (a3).

In a preferred embodiment of the present invention the homecare product composition or the cosmetic or pharmaceutical composition comprises the component (a) which is defined as 1,2-nonanediol and/or 2,3-nonandediol. The components (a) 1,2-nonanediol and/or 2,3-nonandediol belong to the category of alkanediols and are straight chain alkanediols with two functional OH-groups in vicinal positions (alpha,beta-position or beta,gamma-position, respectively) and nine carbon atoms (C9) according to the following general formulae (left: 1,2-nonanediol; right: 2,3-nonanediol):

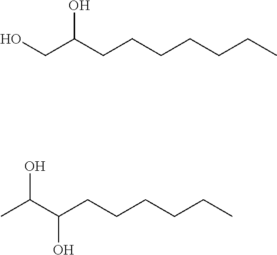

Both compounds 1,2-nonanediol and 2,3-nonandiol exhibit an antimicrobial effect and a malodour coverage effect. Consequently, both compounds are highly suitable as ingredients in compositions for use in malodour reduction or inhibiting malodour development or body odour control or for repelling insects. Preferably, in a homecare product composition or a cosmetic or pharmaceutical composition comprises component (a), wherein component (a) is 1,2-nonanediol and/or 2,3-nonanediol, preferably component (a) is 1,2-nonanediol or a mixture of 1,2-nonanediol and 2,3-nonanediol, and even more preferred component (a) is 1,2-nonanediol.

In the context of the present invention the term “malodour coverage” means that the unpleasant smell of one or more substances or a composition is entirely suppressed or reduced, but the coverage is not achieved by a beneficial intrinsic odour of an added compound. Generally, an unpleasant smell of a substance or a composition is improved by adding a compound exhibiting a malodour coverage effect, however the added compound does not improve the unpleasant smell of the substance or composition due to a beneficial intrinsic odour of the added compound. The added compound is an odour neutral molecule without a beneficial intrinsic odour. Therefore, in the context of the present invention the term “malodour coverage” does not relate to fragrance compounds which only cover an unpleasant smell due to a strong beneficial intrinsic odour.

In the context of the present invention, the term “inhibiting malodour development” means that the formation of an unpleasant smell is entirely suppressed or reduced. For example, the suppression or inhibition of the formation of an unpleasant smell may be achieved by the suppression or inhibition of the production of sweat and/or by the suppression or inhibition of microbial activity.

In the context of the present invention, the term “malodour reduction” means that the formation of an unpleasant smell is entirely suppressed or inhibited and/or an already existing unpleasant smell is entirely suppressed or reduced. For example, the suppression or inhibition of the formation of an unpleasant smell may be achieved by the suppression or inhibition of the production of sweat and/or by the suppression or inhibition of microbial activity. The suppression or reduction of an already existing unpleasant smell is achieved by malodour coverage. Therefore, “malodour reduction” may be achieved by inhibiting malodour development and/or malodour coverage.

In the context of the present invention, the term “body odour control” means that the malodour reduction takes place on a human or mammalian body.

In the context of the present invention, the term “masking insect alluring odours” means that the odours which allure or attract insects such as biting insects are covered or masked via the “malodour coverage” effect of the present invention. For example, the malodour and insect alluring odour of 1-octen-3-ol is covered or masked via the excellent “malodour coverage” effect of the compositions according to the present invention.

In a preferred embodiment of the present invention the homecare product composition or the cosmetic or pharmaceutical composition according to the second aspect of the present invention comprises the component (a) which is defined in a first alternative as at least one linear alkanediol having a carbon chain of 5 to 14 carbon atoms or in a second alternative as a mixture of or comprising at least one first linear alkanediol having a carbon chain of 5 to 14 carbon atoms with one or more second linear alkanediol(s) having a carbon chain of 5 to 14 carbon atoms which is different to the first linear alkanediol.

The component (a) which is defined as at least one linear alkanediol having a carbon chain of 5 to 14 carbon atoms or a mixture of or comprising at least one first linear alkanediol having a carbon chain of 5 to 14 carbon atoms with one or more second linear alkanediol(s) having a carbon chain of 5 to 14 carbon atoms which is different to the first linear alkanediol exhibits an antimicrobial effect and a malodour coverage effect. Consequently, the component (a) is highly suitable as ingredient in compositions for use in malodour reduction or inhibiting malodour development or body odour control or for repelling insects.

Since the first linear alkanediol of the invention can be selected from the same lists and types of compounds as the linear alkanediol according to the first alternative of the second aspect, in the following, reference is made to the linear alkanediol in general, which can also be the first linear alkanediol. In mixtures of first and second linear alkanediols, where these are specifically different, the specific terms “first” and “second” will be used to distinguish the two alkanediol components.

In a first preferred alternative, the component (a) is at least one linear alkanediol having a carbon chain of 5 to 14 carbon atoms.

Moreover, in a second preferred alternative, the component (a) is a mixture of at least one first linear alkanediol having a carbon chain of 5 to 14 carbon atoms with one or more second linear alkanediol(s) having a carbon chain of 5 to 14 carbon atoms which is different to the first linear alkanediol. Preferably, the component (a) is a mixture of one first linear alkanediol with three second linear alkanediol(s) which are different to the first linear alkanediol, more preferably the component (a) is a mixture of one first linear alkanediol with two second linear alkanediol(s) which are different to the first linear alkanediol, most preferably the component (a) is a mixture of one first linear alkanediol with one second linear alkanediol which is different to the first linear alkanediol.

In the present invention the linear alkanediol, the first linear alkanediol and the second linear alkanediol consist of a chain of 5 to 14 carbon atoms joined to each other by single covalent bonds with two OH-functional groups attached to two different carbon atoms in the chain.

The at least one linear alkanediol having a carbon chain of 5 to 14 carbon atoms or the at least one first linear alkanediol having a carbon chain of 5 to 14 carbon atoms in the homecare product composition or the cosmetic or pharmaceutical composition according to the present invention are preferably selected from the group consisting of pentanediol, hexanediol, heptanediol, octanediol, nonanediol, decanediol, undecanediol, dodecanediol, tridecanediol, and mixtures thereof, more preferably selected from the group consisting of heptanediol, octanediol, nonanediol, decanediol, undecanediol, dodecanediol and tridecanediol, even more preferably selected from the group consisting of heptanediol, nonanediol, undecanediol and tridecanediol, still more preferably selected from the group consisting of heptanediol, nonanediol and undecanediol, still more preferably selected from the group consisting of nonanediol and undecanediol, most preferably the at least one linear alkanediol or the at least one first linear alkanediol are a nonanediol.

Thus, in a more preferred variant, the at least one linear alkanediol or the at least one first linear alkanediol is nonanediol.

According to another even more preferred variant the at least one linear alkanediol having a carbon chain of 5 to 14 carbon atoms or the at least one first linear alkanediol having a carbon chain of 5 to 14 carbon atoms is selected from the group consisting of heptanediol, octanediol and nonanediol.

In an alternative, the composition according to the second aspect of the present invention comprises a mixture comprising at least one first linear alkanediol having a carbon chain of 5 to 14 carbon atoms and one or more second linear alkanediol(s) having a carbon chain of 5 to 14 carbon atoms which is/are different from the first linear alkanediol(s).

This means that the alkanediol mixture is a mixture or combination comprising at least one first linear alkanediol having a carbon chain of 5 to 14 carbon atoms and one or more, i.e. two, three, four or more second linear alkanediol(s) having a carbon chain of 5 to 14 carbon atoms. In this alternative, the first linear alkanediol can be any of the alkanediols as described herein for the linear alkanediol in general and in particular for the first alternative of the second aspect.

For example, the mixture can include one first linear alkanediol and one, two, three or more second linear alkanediols; or the mixture can include two first linear alkanediols with one, two, three or more second linear alkanediols, etc. with the proviso, that in each mixture, the first linear alkanediol(s) and the second linear alkanediol(s) are different from each other as specified above.

The phrase “different from each other means” that the first linear alkanediol(s) and the second linear alkanediol(s) in the mixture are either different with regard to the length of their carbon chain, i.e. number of the carbon atoms, and/or with regard to their constitutional isomerism or with regard to their stereoisomerism.

Likewise, the number of the carbon atoms of the first linear alkanediol(s) and the second linear alkanediol(s) in the mixture can also be same. For example, the first linear alkanediol and the second linear alkanediol have a carbon chain of 7 carbon atoms, but the first linear alkanediol and the second linear alkanediol are different with regard to their constitutional isomerism or with regard to their stereoisomerism.

The second linear alkanediol preferably consists of a chain of 5 to 14 carbon atoms joined to each other by single covalent bonds with two OH-functional groups attached to two different carbon atoms in the chain.

The one or more second linear alkanediol(s) having a carbon chain of 5 to 14 carbon atoms in the homecare product composition or the cosmetic or pharmaceutical composition according to the present invention is/are preferably selected from the group consisting of pentanediol, hexanediol, heptanediol, octanediol, nonanediol, decanediol, undecanediol, dodecanediol, tridecanediol and tetradecanediol. Preferably, the at least one/one or more second linear alkanediol is/are selected from the group consisting of pentanediol, hexanediol, heptanediol, octanediol, nonanediol, decanediol, undecanediol, dodecanediol and tridecanediol, more preferably selected from the group consisting of heptanediol, octanediol, nonanediol, decanediol, undecanediol, dodecanediol and tridecanediol, even more preferably selected from the group consisting of heptanediol, nonanediol, undecanediol and tridecanediol, still more preferably selected from the group consisting of heptanediol, nonanediol and undecanediol, most preferably selected from the group consisting of nonanediol and undecanediol.

If both the first and the second linear alkanediol are an alkanediol having the same chain length, then the second linear alkanediol is a different constitutional isomer or stereoisomer from the first linear alkanediol.

The term “alkanediol” within the context of the present invention also includes its constitutional isomers or position isomers. Constitutional isomers are compounds that have the same molecular formula and different connectivity. Position isomers, a particular form of constitutional isomerism, are structural isomers that can be viewed as differing only on the position of a functional group on a parent structure, which in this case is the position of the two alcohol functions.

Depending on the number of the carbon atoms of the carbon chain of the alkanediol, there are various position isomers of the alkanediol: (x,x+1)-constitutional isomers; (x,x+2)-constitutional isomers; (x,x+3)-constitutional isomers; etc. and alpha,omega-constitutional isomers when the alcohol functions are at the terminal ends of the carbon chain, wherein x stands for the number of the carbon atom in the alkanediol chain, to which the OH-groups of the alkanediol are chemically bonded. For example: if x is 1, the two OH-groups of the alkanediol are chemically bonded to the C1 and C2 carbon atoms in the alkanediol chain; if x is 2, the two OH-groups of the alkanediol are chemically bonded to the C2 and C3 carbon atoms in the alkanediol chain, etc.

In the (x,x+1)-constitutional isomers, the two OH-functional groups are vicinal attached to two different adjacent carbon atoms in the chain. In the (x,x+2)-constitutional isomers, the two OH-functional groups are attached to two different carbon atoms in the chain, wherein the two carbon atoms are separated by one C-atom. In the (x,x+3)-constitutional isomers, the two OH-functional groups are attached to two different carbon atoms in the chain where the two carbon atoms are separated by two C-atoms. In the alpha,omega-constitutional isomers, the two functional groups are attached to the first C-atom and to the terminal C-atom.

In a preferred variant of the homecare product composition or the cosmetic or pharmaceutical composition according to the present invention, the linear alkanediol having a carbon chain of 5 to 14 carbon atoms, is preferably a vicinal (x,x+1)-diol, selected from the group consisting of a 1,2-diol, 2,3-diol, 3,4-diol, 4,5-diol and further (x,x+1)-diols, and mixtures thereof, preferably an alpha,beta-1,2-constitutional isomer.

In a preferred variant of the homecare product composition or the cosmetic or pharmaceutical composition according to the present invention, the first linear alkanediol and/or the second linear alkanediol having a carbon chain of 5 to 14 carbon atoms is a vicinal (x,x+1)-diol, selected from the group consisting of a 1,2-diol, 2,3-diol, 3,4-diol, 4,5-diol and further (x,x+1)-diols, and mixtures thereof, preferably an alpha,beta-1,2-constitutional isomer.

In a further preferred variant of the homecare product composition or the cosmetic or pharmaceutical composition according to the present invention, the linear alkanediol having a carbon chain of 5 to 14 carbon atoms, is preferably a non-vicinal (x,x+2)-diol, selected from the group consisting of a 1,3-diol, 2,4-diol, 3,5-diol, and further (x,x+2)-diols or mixtures thereof, preferably an alpha,gamma-1,3-constitutional isomer.

In a preferred variant of the homecare product composition or the cosmetic or pharmaceutical composition according to the present invention, the first linear alkanediol and/or the second linear alkanediol having a carbon chain of 5 to 14 carbon atoms is a non-vicinal (x,x+2)-diol, selected from the group consisting of a 1,3-diol, 2,4-diol, 3,5-diol, 4,6-diol and further (x,x+2)-diols or mixtures thereof, preferably an alpha,gamma-1,3-consitutional isomer.

In a further preferred variant of the homecare product composition or the cosmetic or pharmaceutical composition according to the present invention, the linear alkanediol having a carbon chain of 5 to 14 carbon atoms, is preferably a non-vicinal (x,x+3)-diol, selected form the group consisting of a 1,4-diol, 2,5-diol, and further (x,x+3)-diols or mixtures thereof, preferably an alpha,delta-1,4-constitutional isomer.

In a preferred variant of the homecare product composition or the cosmetic or pharmaceutical composition according to the present invention, the first linear alkanediol and/or the secondary linear alkanediol having a carbon chain of 5 to 14 carbon atoms is a non-vicinal (x,x+3)-diol, selected form the group consisting of a 1,4-diol, 2,5-diol, and further (x,x+3)-diols or mixtures thereof, preferably an alpha,delta-1,4-constitutional isomer.

In a preferred variant of the homecare product composition or the cosmetic or pharmaceutical composition according to the invention, the linear alkanediol or the first linear alkanediol and/or the second linear alkanediol is preferably an alpha,omega-alkanediol, more preferably, 1,7-heptanediol or 1,9-nonanediol or 1,11-undecanediol.

According to the present invention, vicinal (x,x+1)-diols are most preferred, such as alpha,beta or beta,gamma or gamma,delta etc.

In a still preferred variant of the homecare product composition or the cosmetic or pharmaceutical composition according to the present invention, the linear alkanediol and in particular the first linear alkanediol and/or the second linear alkanediol, is a 1,2-alkanediol, a 2,3-alkanediol or a 3,4-alkanediol or mixtures thereof.

In the most preferred variant of the homecare product composition or the cosmetic or pharmaceutical composition according to the present invention the at least one linear alkanediol, the at least one first linear alkanediol and/or the one or more second linear alkanediol(s) is/are a (x,x+1)-constitutional isomer, in particular a 1,2-alkanediol, a 2,3-alkanediol or a 3,4-alkanediol or mixtures thereof, preferably a 1,2-alkanediol or a 2,3-alkanediol or mixtures thereof.

In a preferred variant, the composition according to the second aspect of the present invention comprises a mixture comprising at least one first linear alkanediol having a carbon chain of 5 to 14 carbon atoms and one or more second linear alkanediol(s) having a carbon chain of 5 to 14 carbon atoms wherein the number of the carbon atoms of the first and the second alkanediol is either same or different.

If both, the first and the second alkanediol have the same number of carbon atoms, such an alkanediol combination is herein also referred to as “homo alkanediol mixture” or “homo combination”. For example, the first linear alkanediol and the second linear alkanediol have a carbon chain of 7 carbon atoms, but the first linear alkanediol and the second linear alkanediol are different with regard to their constitutional isomerism or with regard to their stereoisomerism.

If the first and the second alkanediol have a different number of carbon atoms, such an alkanediol combination is herein also referred to as “hetero alkanediol mixture” or “hetero combination”. For example, the first linear alkanediol has a carbon chain of 7 carbon atoms and the second linear alkanediol has a carbon chain of 8 carbon atoms. However, beyond that, the first linear alkanediol and the second linear alkanediol can be different with regard to their constitutional isomerism or with regard to their stereoisomerism.

In a preferred variant of the compositions according to the second aspect of the present invention, the at least one linear alkanediol or the at least one first linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol,
1,2-dodecanediol, 2,3-dodecanediol, 3,4-dodecanediol,
1,2-tridecanediol, 2,3-tridecanediol, 3,4-tridecanediol, and mixtures thereof.

Preferably, the at least one linear alkanediol and the at least one first linear alkanediol are selected from the group consisting of:
1,2-pentanediol, 2,3-pentanediol,
1,2-hexanediol, 2,3-hexanediol,
1,2-heptanediol, 2,3-heptanediol,
1,2-octanediol, 2,3-octanediol,
1,2-nonanediol, 2,3-nonanediol,
1,2-decanediol, 2,3-decanediol,
1,2-undecanediol, 2,3-undecanediol,
1,2-dodecanediol, 2,3-dodecanediol,
1,2-tridecanediol, 2,3-tridecanediol, and mixtures thereof, and preferably the at least one linear alkanediol or the at least one first linear alkanediol is selected from the group consisting of 1,2-pentanediol, 2,3-pentanediol, 1,2-hexanediol, 2,3-hexanediol, 1,2-heptanediol, 2,3-heptanediol, 1,2-octanediol, 2,3-octanediol, 1,2-nonanediol, 2,3-nonanediol, 1,2-undecanediol, 2,3-undecanediol, and mixtures thereof.

According to another preferred variant, of the aforesaid alkanediols, the alpha,beta-constitutional isomers and/or beta,gamma-constitutional isomers are preferred: 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, 2,3-dodecanediol, or 2,3-tridecanediol. Said alkanediols are liquid at a purity of 90 to 99%.

Of the aforesaid liquid alkanediols 1,2-pentanediol, 2,3-pentanediol, 1,2-hexanediol, 2,3-hexanediol, 1,2-heptanediol, 2,3-heptanediol, 2,3-octanediol, 2,3-nonanediol or mixtures of said liquid alkanediols are particularly preferred. Said alkanediols can be easier incorporated into semi-finished products or final products.

In a more preferred variant, the at least one linear alkanediol or the at least one first linear alkanediol is selected from the group consisting of 1,2-alkanediols having an uneven number of carbon atoms of 5 to 13, i.e., 1,2-pentanediol, 1,2-heptanediol, 1,2-nonanediol, 1,2-undecanediol, 1,2-tridecanediol and mixtures thereof.

In a more preferred variant, the linear alkanediol or the first linear alkanediol is selected from the group consisting of 1,2-alkanediols having an uneven number of carbon atoms of 7, 9, 11 and 13, i.e. 1,2-heptanediol, 1,2-nonanediol, 1,2-undecanediol or 1,2-tridecanediol, or is selected from the group consisting of 1,2-alkanediols having an uneven number of carbon atoms of 9 and 11, i.e. 1,2-nonanediol and 1,2-undecanediol, or is selected from the group consisting of 1,2-alkanediols having an uneven number of carbon atoms of 9 and 13, i.e. 1,2-nonanediol and 1,2-tridecanediol, or is selected from the group consisting of 1,2-alkanediols having an uneven number of carbon atoms of 11 and 13, i.e. 1,2-undecanediol and 1,2-tridecanediol.

In a more preferred variant, the at least one linear alkanediol or the at least one first linear alkanediol is selected from the group consisting of 2,3-alkanediols having an uneven number of carbon atoms of 5 to 13, i.e., 2,3-pentanediol, 2,3-heptanediol, 2,3-nonanediol, 2,3-undecanediol, 2,3-tridecanediol and mixtures thereof.

In a still more preferred variant, the linear alkanediol or the first linear alkanediol is selected from the group consisting of 2,3-alkanediols having an uneven number of carbon atoms of 7, 9, 11 and 13, i.e. 2,3-heptanediol, 2,3-nonanediol, 2,3-undecanediol or 2,3-tridecanediol, or is selected from the group consisting of 2,3-alkanediols having an uneven number of carbon atoms of 9 and 11, i.e. 2,3-nonanediol and 2,3-undecanediol, or is selected from the group consisting of 2,3-alkanediols having an uneven number of carbon atoms of 9 and 13, i.e. 2,3-nonanediol and 2,3-tridecanediol, or is selected from the group consisting of 2,3-alkanediols having an uneven number of carbon atoms of 11 and 13, i.e. 2,3-undecanediol and 2,3-tridecanediol.

In a further preferred variant of the composition according to the second aspect of the present invention, the linear alkanediol or the first linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-undecanediol, 2,3-undecanediol, 3,4-undecanediol, and mixtures thereof.

Even more preferred, in the composition according to the second aspect of the present invention, the at least one linear alkanediol or the at least one first linear alkanediol is an alpha,beta or a beta,gamma-diol such as 1,2-heptanediol, 2,3-heptanediol, or a mixture thereof. However, the at least one linear alkanediol or the at least one first linear alkanediol can also preferably be an alpha,beta or a beta,gamma-diol as either 1,2-octanediol, 2,3-octanediol, or a mixture thereof. A mixture comprising 1,2-heptanediol and 1,2-octanediol or a mixture of 1,2-heptanediol and 2,3-octanediol or a mixture of 1,2-octanediol and 2,3-heptanediol is also possible.

In this case, most preferred, the linear alkanediol or the first linear alkanediol is 1,2-heptanediol or 1,2-octanediol.

Even more preferred, the linear alkanediol or the first linear alkanediol is 1,2-nonanediol.

According to another preferred variant the at least one linear alkanediol or the at least one first linear alkanediol is selected from the group consisting of 1,2-heptanediol, 2,3-heptanediol, 1,2-octanediol, 2,3-octanediol, 1,2-nonanediol, 2,3-nonanediol, 1,2-decanediol, 2,3-decanediol, 1,2-undecanediol, 2,3-undecanediol, 1,2-dodecanediol, 2,3-dodecanediol, 1,2-tridecanediol, 2,3-tridecanediol and mixtures thereof, even more preferably selected from the group consisting of 1,2-heptanediol, 2,3-heptanediol, 1,2-nonanediol, 2,3-nonanediol, 1,2-undecanediol 2,3-undecanediol, 1,2-tridecanediol, 2,3-tridecanediol and mixtures thereof, still more preferably selected from the group consisting of 1,2-hepantediol, 2,3-heptanediol, 1,2-nonanediol, 2,3-nonanediol, 1,2-undecanediol, 2,3-undecanediol and mixtures thereof, still more preferably selected from the group consisting of 1,2-nonanediol, 2,3-nonanediol, 1,2-undecanediol, 2,3-undecanediol and mixtures thereof, most preferably the at least one linear alkanediol or the at least one first linear alkanediol are 1,2-nonanediol and/or 2,3-nonanediol.

In a preferred embodiment of the homecare product composition or the cosmetic or pharmaceutical composition according to the present invention, the at least one linear alkanediol or the at least one first linear alkanediol is selected from the group consisting of:
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-undecanediol, 2,3-undecanediol, 3,4-undecanediol, and mixtures thereof.

In a more preferred embodiment of the homecare product composition or the cosmetic or pharmaceutical composition according to the present invention, the at least one linear alkanediol or the at least one first linear alkanediol is selected from the group consisting of:
1,2-nonanediol, 2,3-nonanediol, 3,4-nonanediol,
1,2-undecanediol, 2,3-undecanediol, 3,4-undecanediol, and mixtures thereof.

In a more preferred embodiment of the homecare product composition or the cosmetic or pharmaceutical composition according to the present invention, the at least one linear alkanediol or the at least one first linear alkanediol is selected from the group consisting of:
1,2-heptanediol, 2,3-heptanediol, 3,4-heptanediol,
1,2-nonanediol, 2,3-nonanediol, 3,4-nonanediol, and mixtures thereof.

More preferred, in the composition according to the second aspect of the present invention, the at least one linear alkanediol or the at least one first linear alkanediol is selected from the group consisting of 1,2-nonanediol, 2,3-nonanediol, 3,4-nonanediol, and mixtures thereof.

Preferably, the one or more second linear alkanediol(s) having a carbon chain of 5 to 14 carbon atoms in the homecare product composition or the cosmetic or pharmaceutical composition according to the present invention is selected from the group consisting of 1,2-pentanediol, 2,3-pentanediol, 1,2-hexanediol, 2,3-hexanediol, 1,2-heptanediol, 2,3-heptanediol, 1,2-octanediol, 2,3-octanediol, 1,2-nonanediol, 2,3-nonanediol, 1,2-decanediol, 2,3-decanediol, 1,2-undecanediol, 2,3-undecanediol, 1,2-dodecanediol, 2,3-dodecanediol, 1,2-tridecanediol, 2,3-tridecanediol, 1,2-tetradecanediol, 2,3-tetradecanediol and mixtures thereof, more preferably selected from the group consisting of 1,2-petanediol, 2,3-pentanediol, 1,2-hexanediol, 2,3-hexanediol, 1,2-heptanediol, 2,3-heptanediol, 1,2-octanediol, 2,3-octanediol, 1,2-nonanediol, 2,3-nonanediol, 1,2-decanediol, 2,3-decanediol, 1,2-undecanediol, 2,3-undecanediol, 1,2-dodecanediol 2,3-dodecanediol, 1,2-tridecanediol, 2,3-tridecanediol and mixtures thereof, even more preferably selected from the group consisting of 1,2-heptanediol, 2,3-heptanediol, 1,2-octanediol, 2,3-octanediol, 1,2-nonanediol, 2,3-nonanediol, 1,2-decanediol, 2,3-decanediol, 1,2-undecanediol, 2,3-undecanediol, 1,2-dodecanediol, 2,3-dodecanediol, 1,2-tridecanediol, 2,3-tridecanediol and mixtures thereof, still more preferably selected from the group consisting of 1,2-heptanediol, 2,3-heptanediol, 1,2-nonanediol, 2,3-nonanediol, 1,2-undecanediol, 2,3-undecanediol, 1,2-tridecanediol, 2,3-tridecanediol and mixtures thereof, still more preferably selected from the group consisting of 1,2-heptanediol, 2,3-heptanediol, 1,2-nonanediol, 2,3-nonanediol, 1,2-undecanediol, 2,3-undecanediol and mixtures thereof, most preferably selected from the group consisting of 1,2-nonanediol, 2,3-nonanediol, 1,2-undecanediol, 2,3-undecanediol, and mixtures thereof.

In a more preferred variant, the second linear alkanediol is selected from the group consisting of 1,2-alkanediols having an uneven number of carbon atoms of 5 to 13, i.e. 1,2-pentanediol, 1,2-heptanediol, 1,2-nonanediol, 1,2-undecanediol, 1,2-tridecanediol, and mixtures thereof.

In a more preferred variant, the second linear alkanediol is selected from the group consisting of 1,2-alkanediols having an uneven number of carbon atoms of 7, 9, 11 and 13, i.e. 1,2-heptanediol, 1,2-nonanediol, 1,2-undecanediol and 1,2-tridecanediol, or is selected from the group consisting of 1,2-alkanediols having an uneven number of carbon atoms of 9 and 11, i.e. 1,2-nonanediol and 1,2-undecanediol, or is selected from the group consisting of 1,2-alkanediols having an uneven number of carbon atoms of 9 and 13, i.e. 1,2-nonanediol and 1,2-tridecanediol, or is selected from the group consisting of 1,2-alkanediols having an uneven number of carbon atoms of 11 and 13, i.e. 1,2-undecanediol and 1,2-tridecanediol.

In a more preferred variant, the second linear alkanediol is selected from the group consisting of 1,2-alkanediols having an uneven number of carbon atoms of 9, 11 and 13, i.e. 1,2-nonanediol, 1,2-undecanediol or 1,2-tridecanediol.

In a more preferred variant, the second linear alkanediol is selected from the group consisting of 2,3-alkanediols having an uneven number of carbon atoms of 5 to 13, i.e. 2,3-pentanediol, 2,3-heptanediol, 2,3-nonanediol, 2,3-undecanediol, 2,3-tridecanediol, and mixtures thereof.

In a still more preferred variant, the second linear alkanediol is selected from the group consisting of 2,3-alkanediols having an uneven number of carbon atoms of 7, 9, 11 and 13, i.e. 2,3-heptanediol, 2,3-nonanediol, 2,3-undecanediol and 2,3-tridecanediol, or is selected from the group consisting of 2,3-alkanediols having an uneven number of carbon atoms of 9 and 11, i.e. 2,3-nonanediol and 2,3-undecanediol, or is selected from the group consisting of 2,3-alkanediols having an uneven number of carbon atoms of 9 and 13, i.e. 2,3-nonanediol and 2,3-tridecanediol, or is selected from the group consisting of 2,3-alkanediols having an uneven number of carbon atoms of 11 and 13, i.e. 2,3-undecanediol and 2,3-tridecanediol.

In a still more preferred variant, the second linear alkanediol is selected from the group consisting of 2,3-alkanediols having an uneven number of carbon atoms of 9, 11 and 13, i.e. 2,3-nonanediol, 2,3-undecanediol or 2,3-tridecanediol.

In a preferred embodiment of the homecare product composition or the cosmetic or pharmaceutical composition according to the present invention, the second linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol,
1,2-dodecanediol, 2,3-dodecanediol, 3,4-dodecanediol,
1,2-tridecanediol, 2,3-tridecanediol, 3,4-tridecanediol, and mixtures thereof.

Of the aforesaid alkanediols, the following (x,x+1) constitutional isomers are particularly preferred: 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, 2,3-dodecanediol, or 2,3-tridecanediol. Said alkanediols are liquid at a purity of 90 to 99%.

Of the aforesaid liquid alkanediols 1,2-pentanediol, 2,3-pentanediol, 1,2-hexanediol, 2,3-hexanediol, 1,2-heptanediol, 2,3-heptanediol, 2,3-octanediol, 2,3-nonanediol or mixtures of said liquid alkanediols are particularly preferred. Said alkanediols can be easier incorporated into semi-finished products or final products.

In a more preferred embodiment of the homecare product composition or the cosmetic or pharmaceutical composition according to the present invention, the second linear alkanediol is selected from the group consisting of:
1,2-heptanediol, 2,3-heptanediol, 3,4-heptanediol,
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol,
1,2-dodecanediol, 2,3-dodecanediol, 3,4-dodecanediol,
1,2-tridecanediol, 2,3-tridecanediol, 3,4-tridecanediol, and mixtures thereof.

In an even more preferred embodiment of the homecare product composition or the cosmetic or pharmaceutical composition according to the present invention, the second linear alkanediol is selected from the group consisting of:
1,2-heptanediol, 2,3-heptanediol, 3,4-heptanediol,
1,2-nonanediol, 2,3-nonanediol, 3,4-nonanediol,
1,2-undecanediol, 2,3-undecanediol, 3,4-undecanediol,
1,2-tridecanediol, 2,3-tridecanediol, 3,4-tridecanediol, and mixtures thereof.

In a more preferred variant of the composition according to the second aspect of the present invention, the one or more second linear alkanediol(s) is/are selected from the group consisting of:
1,2-pentanediol, 2,3-pentanediol,
1,2-hexanediol, 2,3-hexanediol,
1,2-heptanediol, 2,3-heptanediol,
1,2-octanediol, 2,3-octanediol,
1,2-nonanediol, 2,3-nonanediol,
1,2-decanediol, 2,3-decanediol,
1,2-undecanediol, 2,3-undecanediol,
1,2-dodecanediol, 2,3-dodecanediol,
1,2-tridecanediol, 2,3-tridecanediol, and mixtures thereof;
   and preferably the one or more second linear alkanediol(s) is/are selected from the group consisting of 1,2-pentanediol, 2,3-pentanediol, 1,2-hexanediol, 2,3-hexanediol, 1,2-heptanediol, 2,3-heptanediol, 1,2-octanediol, 2,3-octanediol, 1,2-nonanediol, 2,3-nonanediol, 1,2-undecanediol, 2,3-undecanediol, and mixtures thereof.

Even more preferred, in the composition according to the second aspect of the present invention, the one or more second linear alkanediol(s) is/are an alpha,beta or a beta,gamma-diol selected from the group consisting of:
1,2-heptanediol, 2,3-heptanediol,
1,2-octanediol, 2,3-octanediol,
1,2-nonanediol, 2,3-nonanediol,
1,2-undecanediol, 2,3-undecanediol, and mixtures thereof.

In a particularly preferred variant, the homecare product composition or the cosmetic or pharmaceutical composition according to the present invention which comprises a mixture or a combination of the at least one first linear alkanediol with one or more second linear alkanediol(s) thus includes any one of the following mixtures/combinations:
1,2-pentanediol in combination with one of 1,2-hexanediol, 1,2-heptanediol, 1,2-octanediol, 1,2-nonanediol, 1,2-decanediol, 1,2-undecanediol, 1,2-dodecanediol, or 1,2-tridecanediol;
1,2-hexanediol in combination with one of 1,2-pentanediol, 1,2-heptanediol, 1,2-octanediol, 1,2-nonanediol, 1,2-decanediol, 1,2-undecanediol, 1,2-dodecanediol, or 1,2-tridecanediol;
1,2-heptanediol in combination with one of 1,2-pentanediol, 1,2-hexanediol, 1,2-octanediol, 1,2-nonanediol, 1,2-decanediol, 1,2-undecanediol, 1,2-dodecanediol, or 1,2-tridecanediol;
1,2-octanediol in combination with one of 1,2-pentanediol, 1,2-hexanediol, 1,2-heptanediol, 1,2-nonanediol, 1,2-decanediol, 1,2-undecanediol, 1,2-dodecanediol, or 1,2-tridecanediol;
1,2-nonanediol in combination with one of 1,2-pentanediol, 1,2-hexanediol, 1,2-heptanediol, 1,2-octanediol, 1,2-decanediol, 1,2-undecanediol, 1,2-dodecanediol, or 1,2-tridecanediol;
1,2-decanediol in combination with one of 1,2-pentanediol, 1,2-hexanediol, 1,2-heptanediol, 1,2-octanediol, 1,2-nonanediol, 1,2-undecanediol, 1,2-dodecanediol, or 1,2-tridecanediol;
1,2-undecandiol in combination with one of 1,2-pentanediol, 1,2-hexanediol, 1,2-heptanediol, 1,2-octanediol, 1,2-nonanediol, 1,2-decanediol, 1,2-dodecanediol, or 1,2-tridecanediol;
1,2-dodecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-tridecanediol;
1,2-tridecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-dodecanediol;
2,3-petanediol in combination with one of 1,2-hexanediol, 1,2-heptanediol, 1,2-octanediol, 1,2-nonanediol, 1,2-decanediol, 1,2-undecanediol, 1,2-dodecanediol, or 1,2-tridecanediol;
2,3-hexanediol in combination with one of 1,2-pentanediol, 1,2-heptanediol, 1,2-octanediol, 1,2-nonanediol, 1,2-decanediol, 1,2-undecanediol, 1,2-dodecanediol, or 1,2-tridecanediol;
2,3-heptanediol in combination with one of 1,2-pentanediol, 1,2-hexanediol, 1,2-octanediol, 1,2-nonanediol, 1,2-decanediol, 1,2-undecanediol, 1,2-dodecanediol, or 1,2-tridecanediol;
2,3-octanediol in combination with one of 1,2-pentanediol, 1,2-hexanediol, 1,2-heptanediol, 1,2-nonanediol, 1,2-decanediol, 1,2-undecanediol, 1,2-dodecanediol, or 1,2-tridecanediol;
2,3-nonanediol in combination with one of 1,2-pentanediol, 1,2-hexanediol, 1,2-heptanediol, 1,2-octanediol, 1,2-decanediol, 1,2-undecanediol, 1,2-dodecanediol, or 1,2-tridecanediol;
2,3-decanediol in combination with one of 1,2-pentanediol, 1,2-hexanediol, 1,2-heptanediol, 1,2-octanediol, 1,2-nonanediol, 1,2-undecanediol, 1,2-dodecanediol, or 1,2-tridecanediol;
2,3-undecandiol in combination with one of 1,2-pentanediol, 1,2-hexanediol, 1,2-heptanediol, 1,2-octanediol, 1,2-nonanediol, 1,2-decanediol, 1,2-dodecanediol, or 1,2-tridecanediol;
2,3-dodecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-tridecanediol; or
2,3-tridecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-dodecanediol;
2,3-pentanediol in combination with one of 2,3-hexanediol, 2,3-heptanediol, 2,3-octanediol, 2,3-nonanediol, 2,3-decanediol, 2,3-undecanediol, 2,3-dodecanediol, or 2,3-tridecanediol;
2,3-hexanediol in combination with one of 2,3-pentanediol, 2,3-heptanediol, 2,3-octanediol, 2,3-nonanediol, 2,3-decanediol, 2,3-undecanediol, 2,3-dodecanediol, or 2,3-tridecanediol;
2,3-heptanediol in combination with one of 2,3-pentanediol, 2,3-hexanediol, 2,3-octanediol, 2,3-nonanediol, 2,3-decanediol, 2,3-undecanediol, 2,3-dodecanediol, or 2,3-tridecanediol;
2,3-octanediol in combination with one of 2,3-pentanediol, 2,3-hexanediol, 2,3-heptanediol, 2,3-nonanediol, 2,3-decanediol, 2,3-undecanediol, 2,3-dodecanediol, or 2,3-tridecanediol;
2,3-nonanediol in combination with one of 2,3-pentanediol, 2,3-hexanediol, 2,3-heptanediol, 2,3-octanediol, 2,3-decanediol, 2,3-undecanediol, 2,3-dodecanediol, or 2,3-tridecanediol;
2,3-decanediol in combination with one of 2,3-pentanediol, 2,3-hexanediol, 2,3-heptanediol, 2,3-octanediol, 2,3-nonanediol, 2,3-undecanediol, 2,3-dodecanediol, or 2,3-tridecanediol;
2,3-undecandiol in combination with one of 2,3-pentanediol, 2,3-hexanediol, 2,3-heptanediol, 2,3-octanediol, 2,3-nonanediol, 2,3-decanediol, 2,3-dodecanediol, or 2,3-tridecanediol;
2,3-dodecanediol in combination with one of 2,3-pentanediol, 2,3-hexanediol, 2,3-heptanediol, 2,3-octanediol, 2,3-nonanediol, 2,3-decanediol, 2,3-undecanediol, or 2,3-tridecanediol; or
2,3-tridecanediol in combination with one of 2,3-pentanediol, 2,3-hexanediol, 2,3-heptanediol, 2,3-octanediol, 2,3-nonanediol, 2,3-decanediol, 2,3-undecanediol, or 2,3-dodecanediol.

Particularly favourable are the mixtures or combinations of 1,2-heptanediol with 1,2-nonanediol and 1,2-nonanediol with 1,2-undecanediol.

Further favourable are the mixtures of 1,2-heptanediol with 2,3-octanediol.

The homecare product composition or the cosmetic or pharmaceutical composition according to the present invention preferably comprises a mixture of at least one first linear alkanediol with one or more second linear alkanediol(s) which is selected from the group consisting of
a mixture comprising 1,2-pentanediol and 2,3-pentanediol;
a mixture comprising 1,2-hexanediol and 2,3-hexanediol;
a mixture comprising 1,2-heptanediol and 2,3-heptanediol;
a mixture comprising 1,2-heptanediol and 1,2-nonanediol;
a mixture comprising 1,2-heptanediol and 2,3-nonanediol;
a mixture comprising 1,2-heptanediol and 1,2-undecanediol;
a mixture comprising 1,2-heptanediol and 2,3-undecanediol;
a mixture comprising 2,3-heptanediol and 1,2-nonanediol;
a mixture comprising 2,3-heptanediol and 2,3-nonanediol;
a mixture comprising 2,3-heptanediol and 1,2-undecanediol;
a mixture comprising 2,3-heptanediol and 2,3-undecanediol;
a mixture comprising 1,2-octanediol and 2,3-octanediol;
a mixture comprising 1,2-nonanediol and 2,3-nonanediol;
a mixture comprising 1,2-nonanediol and 1,2-undecanediol;
a mixture comprising 1,2-nonanediol and 2,3-undecanediol;
a mixture comprising 2,3-nonanediol and 1,2-undecanediol;
a mixture comprising 2,3-nonanediol and 2,3-undecanediol;
a mixture comprising 1,2-decanediol and 2,3-decanediol;
a mixture comprising 1,2-undecanediol and 2,3-undecanediol;
a mixture comprising 1,2-dodecanediol and 2,3-dodecanediol; and
a mixture comprising 1,2-tridecanediol and 2,3-tridecanediol.

In a particular preferred variant, the homecare product composition or the cosmetic or pharmaceutical composition according to the present invention which comprises a mixture or a combination of the at least one first linear alkanediol with one or more second linear alkanediol thus may include any of the following mixtures/combinations:
a mixture comprising 1,2-heptanediol and 2,3-heptanediol,
a mixture comprising 1,2-heptanediol and 1,2-nonanediol,
a mixture comprising 1,2-heptanediol and 1,2-undecanediol,
a mixture comprising 2,3-heptanediol and 1,2-nonanediol,
a mixture comprising 2,3-heptanediol and 1,2-undecanediol,
a mixture comprising 2,3-heptanediol and 2,3-nonanediol,
a mixture comprising 2,3-heptanediol and 2,3-undecanediol,
a mixture comprising 1,2-nonanediol and 2,3-nonanediol,
a mixture comprising 1,2-nonanediol and 1,2-undecanediol,
a mixture comprising 1,2-nonanediol and 2,3-undecanediol,
a mixture comprising 2,3-nonanediol and 1,2-undecanediol,
a mixture comprising 2,3-nonanediol and 2,3-undecanediol,
a mixture comprising 1,2-undecanediol and 2,3-undecanediol.

In a particularly preferred variant, the composition according to the present invention comprises a mixture or a combination of the at least one first linear alkanediol with one or more second linear alkanediol which includes any one of the following mixtures/combinations:
a mixture comprising 1,2-pentanediol and 2,3-pentanediol;
a mixture comprising 1,2-hexanediol and 2,3-hexanediol;
a mixture comprising 1,2-heptanediol and 2,3-heptanediol;
a mixture comprising 1,2-octanediol and 2,3-octanediol;
a mixture comprising 1,2-nonanediol and 2,3-nonanediol;
a mixture comprising 1,2-decanediol and 2,3-decanediol;
a mixture comprising 1,2-undecanediol and 2,3-undecanediol;
a mixture comprising 1,2-dodecanediol and 2,3-dodecanediol; or
a mixture comprising 1,2-tridecanediol and 2,3-tridecanediol.

In said homo alkanediol mixtures the first linear alkanediol and the second linear alkanediol have the same number of carbon atoms, but differ in their isomeric structure.

In a preferred embodiment, the homecare product composition or the cosmetic or pharmaceutical composition comprises the component (a) wherein the linear alkanediol or the at least one first linear alkanediol, is selected from the group consisting of:
1,2-pentanediol, 2,3-pentanediol,
1,2-hexanediol, 2,3-hexanediol,
1,2-heptanediol, 2,3-heptanediol,
1,2-octanediol, 2,3-octanediol,
1,2-nonanediol, 2,3-nonanediol,
1,2-decanediol, 2,3-decanediol,
1,2-undecanediol, 2,3-undecanediol,
1,2-dodecanediol, 2,3-dodecanediol,
1,2-tridecanediol, 2,3-tridecanediol, and mixtures thereof;
   preferably wherein the at least one linear alkanediol or the at least one first linear alkanediol is selected from the group consisting of: 1,2-pentanediol, 2,3-pentanediol, 1,2-hexanediol, 2,3-hexanediol, 1,2-heptanediol, 2,3-heptanediol, 1,2-octanediol, 2,3-octanediol, 1,2-nonanediol, 2,3-nonanediol, 1,2-undecanediol, 2,3-undecanediol, and mixtures thereof;
   and/or
   wherein the one or more second linear alkanediol(s) is/are selected from the group consisting of:
1,2-pentanediol, 2,3-pentanediol,
1,2-hexanediol, 2,3-hexanediol,
1,2-heptanediol, 2,3-heptanediol,
1,2-octanediol, 2,3-octanediol,
1,2-nonanediol, 2,3-nonanediol,
1,2-decanediol, 2,3-decanediol,
1,2-undecanediol, 2,3-undecanediol,
1,2-dodecanediol, 2,3-dodecanediol,
1,2-tridecanediol, 2,3-tridecanediol, and mixtures thereof;
   preferably wherein the one or more second linear alkanediol(s) is/are selected from the group consisting of: 1,2-pentanediol, 2,3-pentanediol, 1,2-hexanediol, 2,3-hexanediol, 1,2-heptanediol, 2,3-heptanediol, 1,2-octanediol, 2,3-octanediol, 1,2-nonanediol, 2,3-nonanediol, 1,2-undecanediol, 2,3-undecanediol, and mixtures thereof.

The above specified homo alkanediol mixtures according to the second aspect of the present invention, comprising a 1,2-alkanediol and a 2,3-alkanediol having the same number of carbon atoms are characterized by an improved synergistical antimicrobial efficacy against a broad spectrum of microorganisms compared to their respective single alkanediol substances, allow for a synergistic malodour coverage effect, and are especially useful for malodour reduction, inhibiting malodour development and body odour control based on said synergistic malodour coverage effect which is considerably increased compared to the single alkanediol substances.

The afore-specified homo alkanediol mixtures including a 1,2-alkanediol and the corresponding 2,3-alkanediol in combination with a cooling component display a remarkably synergistic activity and are clearly superior to the individually corresponding 1,2-alkanediols or 2,3-alkanediols, respectively, and having the same concentration.

However, it was further found that mixtures of different alkanediols in different ratios, i.e. compositions comprising an 1,2-alkanediol and an 2,3-alkanediol differing in their chain lengths (hetero combination) according to the present invention, show a synergistic malodour coverage effect. Thereby, already small portions of the 2,3-compound are sufficient in order to achieve a pronounced advantageous effect and to efficiently cover unpleasant malodour.

Preferably, the composition according to the present invention comprises a mixture or combination of at least one first linear alkanediol with one or more second linear alkanediol(s) which is selected from the group consisting of:
a mixture comprising 1,2-pentanediol and 2,3-pentanediol;
a mixture comprising 1,2-hexanediol and 2,3-hexanediol;
a mixture comprising 1,2-heptanediol and 2,3-heptanediol;
a mixture comprising 1,2-heptanediol and 2,3-octanediol;
a mixture comprising 1,2-heptanediol and 1,2-nonanediol;
a mixture comprising 1,2-heptanediol and 2,3-nonanediol;
a mixture comprising 1,2-heptanediol and 1,2-undecanediol;
a mixture comprising 1,2-heptanediol and 2,3-undecanediol;
a mixture comprising 2,3-heptanediol and 1,2-nonanediol;
a mixture comprising 2,3-heptanediol and 2,3-nonanediol;
a mixture comprising 2,3-heptanediol and 1,2-undecanediol;
a mixture comprising 2,3-heptanediol and 2,3-undecanediol;
a mixture comprising 1,2-octanediol and 2,3-octanediol;
a mixture comprising 1,2-nonanediol and 2,3-nonanediol;
a mixture comprising 1,2-nonanediol and 1,2-undecanediol;
a mixture comprising 1,2-nonanediol and 2,3-undecanediol;
a mixture comprising 2,3-nonanediol and 1,2-undecanediol;
a mixture comprising 2,3-nonanediol and 2,3-undecanediol;
a mixture comprising 1,2-decanediol and 2,3-decanediol;
a mixture comprising 1,2-undecanediol and 2,3-undecanediol;
a mixture comprising 1,2-dodecanediol and 2,3-dodecanediol; and
a mixture comprising 1,2-tridecanediol and 2,3-tridecanediol.

In a further preferred embodiment, the homecare product composition or the cosmetic or pharmaceutical composition comprises the component (a) wherein the linear alkanediol or the at least one first linear alkanediol, is selected from the group consisting of:
1,2-heptanediol, 2,3-heptanediol,
1,2-octanediol, 2,3-octanediol;
1,2-nonanediol, 2,3-nonanediol,
1,2-decanediol, 2,3-decanediol,
1,2-undecanediol, 2,3-undecanediol,
1,2-dodecanediol, 2,3-dodecanediol,
1,2-tridecanediol, 2,3-tridecanediol, and mixtures thereof;
   and/or
   wherein the one or more second linear alkanediol(s) is/are selected from the group consisting of:
1,2-heptanediol, 2,3-heptanediol,
1,2-octanediol, 2,3-octanediol;
1,2-nonanediol, 2,3-nonanediol,
1,2-decanediol, 2,3-decanediol,
1,2-undecanediol, 2,3-undecanediol,
1,2-dodecanediol, 2,3-dodecanediol,
1,2-tridecanediol, 2,3-tridecanediol, and mixtures thereof;
   in particular wherein the linear alkanediol and/or the first linear alkanediol and/or the one or more second linear alkanediol(s) is/are selected from the group consisting of 1,2-heptanediol, 2,3-heptanediol, 1,2-octanediol, 2,3-octanediol, 1,2-nonanediol, 2,3-nonanediol, 1,2-undecanediol, 2,3-undecanediol, 1,2-tridecanediol, and mixtures thereof;
   and/or
   wherein the mixture of at least one first linear alkanediol with one or more second linear alkanediol(s) is selected from the group consisting of
   a mixture comprising 1,2-heptanediol and 2,3-heptanediol;
   a mixture comprising 1,2-heptanediol and 2,3-octanediol;
   a mixture comprising 1,2-heptanediol and 1,2-nonanediol;
   a mixture comprising 1,2-heptanediol and 2,3-nonanediol;
   a mixture comprising 1,2-heptanediol and 1,2-undecanediol;
   a mixture comprising 1,2-heptanediol and 2,3-undecanediol;
   a mixture comprising 2,3-heptanediol and 1,2-nonanediol;
   a mixture comprising 2,3-heptanediol and 2,3-nonanediol;
   a mixture comprising 2,3-heptanediol and 1,2-undecanediol;
   a mixture comprising 2,3-heptanediol and 2,3-undecanediol;
   a mixture comprising 1,2-nonanediol and 2,3-nonanediol;
   a mixture comprising 1,2-nonanediol and 1,2-undecanediol;
   a mixture comprising 1,2-nonanediol and 2,3-undecanediol;
   a mixture comprising 2,3-nonanediol and 1,2-undecanediol;
   a mixture comprising 2,3-nonanediol and 2,3-undecanediol;
   a mixture comprising 1,2-undecanediol and 2,3-undecanediol;
   a mixture comprising 1,2-tridecanediol and 2,3-tridecanediol.

In an even more preferred embodiment, the homecare product composition or the cosmetic or pharmaceutical composition comprises the component (a) wherein the linear alkanediol or the at least one first linear alkanediol, is selected from the group consisting of:
1,2-heptanediol, 2,3-heptanediol,
1,2-nonanediol, 2,3-nonanediol,
1,2-undecanediol, 2,3-undecanediol,
1,2-tridecanediol, 2,3-tridecanediol, and mixtures thereof;
   and/or
   wherein the one or more second linear alkanediol(s) is/are selected from the group consisting of:
1,2-heptanediol, 2,3-heptanediol,
1,2-nonanediol, 2,3-nonanediol,
1,2-undecanediol, 2,3-undecanediol,
1,2-tridecanediol, 2,3-tridecanediol, and mixtures thereof;
   in particular wherein the linear alkanediol and/or the first linear alkanediol and/or the one or more second linear alkanediol(s) is/are selected from the group consisting of 1,2-heptanediol, 2,3-heptanediol, 1,2-nonanediol, 2,3-nonanediol, 1,2-undecanediol, 2,3-undecanediol, 1,2-tridecanediol, and mixtures thereof;
   and/or
   wherein the mixture of at least one first linear alkanediol with one or more second linear alkanediol(s) is selected from the group consisting of:
   a mixture comprising 1,2-heptanediol and 2,3-heptanediol;
   a mixture comprising 1,2-heptanediol and 1,2-nonanediol;
   a mixture comprising 1,2-heptanediol and 2,3-nonanediol;
   a mixture comprising 1,2-heptanediol and 1,2-undecanediol;
   a mixture comprising 1,2-heptanediol and 2,3-undecanediol;
   a mixture comprising 2,3-heptanediol and 1,2-nonanediol;
   a mixture comprising 2,3-heptanediol and 2,3-nonanediol;
   a mixture comprising 2,3-heptanediol and 1,2-undecanediol;
   a mixture comprising 2,3-heptanediol and 2,3-undecanediol;
   a mixture comprising 1,2-nonanediol and 2,3-nonanediol;
   a mixture comprising 1,2-nonanediol and 1,2-undecanediol;
   a mixture comprising 1,2-nonanediol and 2,3-undecanediol;
   a mixture comprising 2,3-nonanediol and 1,2-undecanediol;
   a mixture comprising 2,3-nonanediol and 2,3-undecanediol;
   a mixture comprising 1,2-undecanediol and 2,3-undecanediol;
   a mixture comprising 1,2-tridecanediol and 2,3-tridecanediol.

In a still more preferred embodiment, the homecare product composition or the cosmetic or pharmaceutical composition comprises the component (a) wherein the linear alkanediol or the at least one first linear alkanediol, is selected from the group consisting of:
1,2-heptanediol, 2,3-heptanediol,
1,2-nonanediol, 2,3-nonanediol,
1,2-undecanediol, 2,3-undecanediol, and mixtures thereof;
   and/or
   wherein the one or more second linear alkanediol(s) is/are selected from the group consisting of:
1,2-heptanediol, 2,3-heptanediol,
1,2-nonanediol, 2,3-nonanediol,
1,2-undecanediol, 2,3-undecanediol, and mixtures thereof;
   in particular wherein the linear alkanediol and/or the first linear alkanediol and/or the one or more second linear alkanediol(s) is/are selected from the group consisting of 1,2-heptanediol, 2,3-heptanediol, 1,2-nonanediol, 2,3-nonanediol, 1,2-undecanediol, 2,3-undecanediol, and mixtures thereof;
   and/or
   wherein the mixture of at least one first linear alkanediol with one or more second linear alkanediol(s) is selected from the group consisting of:
   a mixture comprising 1,2-heptanediol and 2,3-heptanediol;
   a mixture comprising 1,2-heptanediol and 1,2-nonanediol;
   a mixture comprising 1,2-heptanediol and 2,3-nonanediol;
   a mixture comprising 1,2-heptanediol and 1,2-undecanediol;
   a mixture comprising 1,2-heptanediol and 2,3-undecanediol;
   a mixture comprising 2,3-heptanediol and 1,2-nonanediol;
   a mixture comprising 2,3-heptanediol and 2,3-nonanediol;
   a mixture comprising 2,3-heptanediol and 1,2-undecanediol;
   a mixture comprising 2,3-heptanediol and 2,3-undecanediol;
   a mixture comprising 1,2-nonanediol and 2,3-nonanediol;
   a mixture comprising 1,2-nonanediol and 1,2-undecanediol;
   a mixture comprising 1,2-nonanediol and 2,3-undecanediol;
   a mixture comprising 2,3-nonanediol and 1,2-undecanediol;
   a mixture comprising 2,3-nonanediol and 2,3-undecanediol;
   a mixture comprising 1,2-undecanediol and 2,3-undecanediol.

In the most preferred embodiment, the homecare product composition or the cosmetic or pharmaceutical composition comprises the component (a) wherein the linear alkanediol or the at least one first linear alkanediol, is selected from the group consisting of:
1,2-nonanediol, 2,3-nonanediol,
1,2-undecanediol, 2,3-undecanediol, and mixtures thereof;
   and/or
   wherein the one or more second linear alkanediol(s) is/are selected from the group consisting of:
1,2-nonanediol, 2,3-nonanediol,
1,2-undecanediol, 2,3-undecanediol, and mixtures thereof;
   in particular wherein the linear alkanediol and/or the first linear alkanediol and/or the one or more second linear alkanediol(s) is/are selected from the group consisting of 1,2-nonanediol, 2,3-nonanediol, 1,2-undecanediol, 2,3-undecanediol, and mixtures thereof;
   and/or
   wherein the mixture of at least one first linear alkanediol with one or more second linear alkanediol(s) is selected from the group consisting of
   a mixture comprising 1,2-nonanediol and 2,3-nonanediol;
   a mixture comprising 1,2-nonanediol and 1,2-undecanediol;
   a mixture comprising 1,2-nonanediol and 2,3-undecanediol;
   a mixture comprising 2,3-nonanediol and 1,2-undecanediol;
   a mixture comprising 2,3-nonanediol and 2,3-undecanediol;
   a mixture comprising 1,2-undecanediol and 2,3-undecanediol.

In addition, advantages hetero combinations of alkanediol mixtures are for example, the hetero alkanediol mixtures comprising 1,2-hexanediol and 2,3-octanediol or the hetero alkanediol mixture comprising 1,2-octanediol and 2,3-hexanediol or the hetero alkanediol mixture comprising 1,2-octanediol and 2,3-heptanediol showing a synergistic antimicrobial effect/malodour reduction compared to their corresponding single 1,2-alkanediol or 2,3-alkanediol substances, namely 1,2-hexanediol or 1,2-octanediol or 2,3-octanediol or 2,3-hexanediol or 2,3-heptanediol.

Said synergistic antimicrobial effect/malodour reduction or action is found for the hetero mixture including 1,2-hexanediol and 2,3-octanediol when used in combination with a cooling substance.

Furthermore, a distinct malodour reducing effect can also be observed for the following specified alkanediol mixtures, namely a mixture of alkanediols including 1,2-pentanediol and 2,3-hexanediol; or a mixture including 1,2-pentanediol and 1,2-heptanediol; or a mixture including 1,2-pentanediol and 2,3-heptanediol; or a mixture including 1,2-pentanediol and 2,3-octanediol; or a mixture including 1,2-pentanediol and 1,2-nonanediol; or a mixture including 1,2-pentanediol and 2,3-nonanediol when used in combination with a cooling agent.

Furthermore, a distinct malodour reducing effect can also be observed for the following specified alkanediol mixtures when used in combination with a cooling agent, namely a mixture of alkanediols including 1,2-heptanediol and 1,2-octanediol or a mixture of alkanediols including 1,2-heptanediol and 2,3-octanediol or a mixture of alkanediols including 1,2-heptanediol and 1,2-nonanediol or a mixture or a combination of alkanediols including 1,2-heptanediol and 2,3-nonanediol.

In a preferred variant according to the second aspect of the present invention in the composition the linear alkanediol is a 2,3-alkandiol, preferably it is a 2,3-alkanediol selected from the group consisting of 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, and mixtures thereof.

The advantage of employing a 2,3-alkandiol lies not only in the malodour reducing effect, but also in the balance of other properties such as formulability in a cosmetic or pharmaceutical formulation and other solubility factors. The combination of these advantages makes the use of these 2,3-alkanediols particularly effective. This is strikingly effective for 2,3-alkandiols with longer chain lengths such as C8 to C12, preferably C8 to C10.

In a preferred variant according to the second aspect of the present invention in the cosmetic or pharmaceutical composition or the homecare product composition the linear alkanediol is selected from the group consisting of 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, and mixtures thereof.

In a more preferred variant of the cosmetic or pharmaceutical composition or the homecare product composition according to the second aspect of the present invention, the linear alkanediol is selected from the group consisting of 2,3-pentanediol, 2,3-hexanediol, 2,3-heptanediol, 2,3-octanediol, 2,3-nonanediol, and mixtures thereof.

Surprisingly, the use of 2,3-pentanediol, 2,3-hexanediol, 2,3-heptanediol, 2,3-octanediol, 2,3-nonanediol, 2,3-decanediol, 2,3-undecanediol, 2,3-dodecanediol and 2,3-tridecanediol in combination with a cooling agent results in an enhanced, i.e. synergistical malodour reducing effect as described herein.

In addition, the 2,3-alkanediols as specified above, have the benefit to be liquids, whereby their incorporation in semi-finished products or final products can be facilitated. Additionally, due to its better solubility, the 2,3-alkanediols can be furnished in a higher concentration in the final formulation.

Hence, a good compromise can be achieved when the active malodour reducing mixture is combined in such a way as to maintain the malodour reducing effect, while improving an improved processability in formulations. The above specified 2,3-alkanediols solve this balancing problem over the solid 1,2-alkanediol substances.

The homecare product composition or cosmetic or pharmaceutical composition according to the present invention preferably comprises a combination or mixture of:
1,2-heptanediol and 1,2-octanediol; or
1,2-heptanediol and 2,3-heptanediol; or
1,2-octanediol and 2,3-octanediol; or
1,2-nonanediol and 2,3-nonanediol,
   showing pronounced synergistic malodour reduction properties.

The boosting effect of the above described alkanediols or alkanediol mixtures has the benefit, that lower concentrations are needed, in order to efficiently reduce malodour in cosmetic or pharmaceutical compositions or homecare product compositions.

Said alkanediols are liquid at a purity of 90 to 99%.

The term “alkanediol” within the context of the present invention also includes its stereoisomers. Stereoisomers are molecules that have the same molecular formula and differ only in how their atoms are arranged in three-dimensional space. In accordance with said definition, the at least one linear alkanediol having a carbon chain of 5 to 14 carbon atoms and the at least one first linear alkanediol having a carbon chain of 5 to 14 carbon atoms or the one or more second linear alkanediol having a carbon chain of 5 to 14 carbon atoms as described above in detail encompass the following stereoisomers:

1,2-Alkanediol Stereoisomers:
(2S)-pentane-1,2-diol,
(2S)-hexane-1,2-diol,
(2S)-heptane-1,2-diol,
(2S)-octane-1,2-diol,
(2S)-nonane-1,2-diol,
(2S)-decane-1,2-diol,
(2S)-undecane-1,2-diol
(2S)-dodecane-1,2-diol,
(2S)-tridecane-1,2-diol,
(2S)-tetradecane-1,2-diol,
(2R)-pentane-1,2-diol,
(2R)-hexane-1,2-diol,
(2R)-heptane-1,2-diol,
(2R)-octane-1,2-diol,
(2R)-nonane-1,2-diol,
(2R)-decane-1,2-diol,
(2R)-undecane-1,2-diol,
(2R)-dodecane-1,2-diol,
(2R)-tridecane-1,2-diol,
(2R)-tetradecane-1,2-diol,

2,3-Alkanediol Stereoisomers:
(2S,3S)-pentane-2,3-diol,
(2S,3S)-hexane-2,3-diol,
(2S,3S)-heptane-2,3-diol,
(2S,3S)-octane-2,3-diol,
(2S,3S)-nonane-2,3-diol,
(2S,3S)-decane-2,3-diol,
(2S,3S)-undecane-2,3-diol
(2S,3S)-dodecane-2,3-diol,
(2S,3S)-tridecane-2,3-diol,
(2S,3S)-tetradecane-2,3-diol,
(2R,3R)-pentane-2,3-diol,
(2R,3R)-hexane-2,3-diol,
(2R,3R)-heptane-2,3-diol,
(2R,3R)-octane-2,3-diol,
(2R,3R)-nonane-2,3-diol,
(2R,3R)-decane-2,3-diol,
(2R,3R)-undecane-2,3-diol,
(2R,3R)-dodecane-2,3-diol,
(2R,3R)-tridecane-2,3-diol,
(2R,3R)-tetradecane-2,3-diol,
(2S,3R)-pentane-2,3-diol,
(2S,3R)-hexane-2,3-diol,
(2S,3R)-heptane-2,3-diol,
(2S,3R)-octane-2,3-diol,
(2S,3R)-nonane-2,3-diol,
(2S,3R)-decane-2,3-diol,
(2S,3R)-undecane-2,3-diol,
(2S,3R)-dodecane-2,3-diol,
(2S,3R)-tridecane-2,3-diol,
(2S,3R)-tetradecane-2,3-diol,
(2R,3S)-pentane-2,3-diol,
(2R,3S)-hexane-2,3-diol,
(2R,3S)-heptane-2,3-diol,
(2R,3S)-octane-2,3-diol,
(2R,3S)-nonane-2,3-diol,
(2R,3S)-decane-2,3-diol,
(2R,3S)-undecane-2,3-diol,
(2R,3S)-dodecane-2,3-diol,
(2R,3S)-tridecane-2,3-diol, and
(2R,3S)-tetradecane-2,3-diol.

3,4-Alkanediol Stereoisomers:
(3R,4R)-hexane-3,4-diol,
(3R,4R)-heptane-3,4-diol,
(3R,4R)-octane-3,4-diol,
(3R,4R)-nonane-3,4-diol,
(3R,4R)-decane-3,4-diol,
(3R,4R)-undecane-3,4-diol,
(3R,4R)-dodecane-3,4-diol,
(3R,4R)-tridecane-3,4-diol,
(3R,4R)-tetradecane-3,4-diol,
(3S,4S)-hexane-3,4-diol,
(3S,4S)-heptane-3,4-diol,
(3S,4S)-octane-3,4-diol,
(3S,4S)-nonane-3,4-diol,
(3S,4S)-decane-3,4-diol,
(3S,4S)-undecane-3,4-diol,
(3S,4S)-dodecane-3,4-diol,
(3S,4S)-tridecane-3,4-diol,
(3S,4S)-tetradecane-3,4-diol,
(3R,4S)-hexane-3,4-diol,
(3R,4S)-heptane-3,4-diol,
(3R,4S)-octane-3,4-diol,
(3R,4S)-nonane-3,4-diol,
(3R,4S)-decane-3,4-diol,
(3R,4S)-undecane-3,4-diol,
(3R,4S)-dodecane-3,4-diol,
(3R,4S)-tridecane-3,4-diol,
(3R,4S)-tetradecane-3,4-diol,
(3S,4R)-hexane-3,4-diol,
(3S,4R)-heptane-3,4-diol,
(3S,4R)-octane-3,4-diol,
(3S,4R)-nonane-3,4-diol,
(3S,4R)-decane-3,4-diol,
(3S,4R)-undecane-3,4-diol,
(3S,4R)-dodecane-3,4-diol,
(3S,4R)-tridecane-3,4-diol, and
(3S,4R)-tetradecane-3,4-diol.

In the context of the present text, the terms “1,2-diol”, “2,3-diol” or “3,4-diol” includes both the corresponding S-configured enantiomers and also the R-enantiomeras well as arbitrary mixtures of these S- and R-configured enantiomers, i.e. mixtures of racemates of the respective diols.

The alkanediol mixture of the composition according to the first aspect of the present invention comprises the 1,2-nonanediol and the 2,3-nonanediol in a ratio in a range of 50:50 to 99.9:0.1, preferably in a ratio in a range of 75:25 to 99:1, more preferred in a ratio in a range of 80:20 to 98:2 or still more preferred in a ratio in a range of 90:10 to 95:5.

In the composition according to the first aspect of the present invention used in a cosmetic or pharmaceutical composition 1,2-nonanediol and 2,3-nonanediol are comprised in the alkanediol mixture preferably in a ratio in a range of 98:2 to 99.9:0.1.

In the composition according to the first aspect of the present invention used in a homecare product 1,2-nonanediol and 2,3-nonanediol are comprised in the alkanediol mixture preferably in a ratio in a range of 95:5 to 99.9:0.1.

Preferably, 1,2-nonanediol and 2,3-nonanediol are comprised in the alkanediol mixture of the composition according to the first aspect of the present invention in a ratio in a range of ≥95:≤5, more preferred in a ratio of ≥96:≤4; still more preferred in a ratio of ≥97:≤3, and most preferred in a ratio of ≥98:≤2.

Most of the alkanediol mixtures of the composition according to the first aspect of the present invention comprises 1,2-nonanediol and 2,3-nonanediol in a ratio in a range of ≥95:≤5, including the ratios ≥95.5:≤4.5; ≥96:≤4; ≥96.5:≤3.5; ≥97:≤3; ≥97.5:2.5 and ≥98.0:≤2.0.

Even more preferred the alkanediol mixture of the composition according to the first aspect of the present invention comprises 1,2-nonanediol and 2,3-nonanediol in a ratio in a range of ≥98:≤2, including the ratios of ≥98.1:≤1.9; ≥98.2:≤1.8; ≥98.3:≤1.7; ≥98.4:≤1.6; ≥98.5:≤1.5; ≥98.6:≤1.4; ≥98.7:≤1.3; ≥98.8:≤1.2; ≥98.9:≤1.1; ≥99:≤1.0; ≥99.1:≤0.9; ≥99.2:≤0.8; ≥99.3:≤0.7; ≥99.4:≤0.6; ≥99.5:≤0.5; ≥99.6:≤0.4; ≥99.7:≤0.3; ≥99.8:≤0.2 and ≥99.9:≤0.1.

The at least one first linear alkanediol and the one or more second linear alkanediol(s) as defined in detail above are present in the alkanediol mixture of the composition according to the second aspect of the present invention in a ratio in a range of 50:50 to 99.9:0.1, preferably in a ratio in a range of 75:25 to 99:1, more preferred in a ratio in a range of 80:20 to 98:2, still more preferred in a ratio in a range of 90:10 to 95:5.

In the composition according to the second aspect of the present invention used in a cosmetic or pharmaceutical composition, the first linear alkanediol and the second linear alkanediol are comprised in the alkanediol mixture preferably in a ratio in a range of 98:2 to 99.9:0.1

In the composition according to the second aspect of the present invention used in a homecare product, the first linear alkanediol and the second alkanediol are comprised in the alkanediol mixture preferably in a ratio in a range of 95:5 to 99.9:0.1.

Preferably, the first linear alkanediol and the second alkanediol are comprised in the alkanediol mixture of the composition according to the second aspect of the present invention in a ratio in a range of ≥95:≤5, more preferred in a ratio of ≥96:≤4; still more preferred in a ratio of ≥97:≤3, and most preferred in a ratio of ≥98:≤2.

Most of the alkanediol mixture of the composition according to the second aspect of the present invention comprises the first linear alkanediol and the second linear alkanediol in a ratio in a range of ≥95:≤5, including the ratios ≥95.5:≤4.5; ≥96:≤4; ≥96.5:≤3.5; ≥97:≤3; ≥97.5:≤2.5 and ≥98.0:≤2.0.

Even more preferred the alkanediol mixture of the composition according to the second aspect of the present invention comprises the first linear alkanediol and the second alkanediol in a ratio in a range of ≥98:≤2, including the ratios of ≥98.1:≤1.9; ≥98.2:≤1.8; ≥98.3:≤1.7; ≥98.4:≤1.6; ≥98.5:≤1.5; ≥98.6:≤1.4; ≥98.7:≤1.3; ≥98.8:≤1.2; ≥98.9:≤1.1; ≥99:≤1.0; ≥99.1:≤0.9; ≥99.2:≤0.8; ≥99.3:≤0.7; ≥99.4:≤0.6; ≥99.5:≤0.5; ≥99.6:≤0.4; ≥99.7:≤0.3; ≥99.8:≤0.2 and ≥99.9:≤0.1.

In a more advantageous variant according to the second aspect of the present invention, in the mixtures comprising a combination of a 1,2-alkanediol as first linear alkanediol and the corresponding 2,3-alkanediol as second linear alkanediol, such as
1,2-pentanediol and 2,3-pentanediol, or
1,2-hexanediol and 2,3-hexanediol, or
1,2-heptanediol and 2,3-heptanediol, or
1,2-octanediol and 2,3-octanediol, or
1,2-nonanediol and 2,3-nonanediol, or
1,2-decanediol and 2,2-decanediol, or
1,2-undecanediol and 2,3-undecenaediol, or
1,2-dodecanediol and 2,3-dodecanediol, or
1,2-tridecanediol and 2,3-tridecanediol,
   wherein the first linear alkanediol and the second linear alkanediol are present in a ratio in a range of 50:50 to 99.9:0.1, preferably in a ratio in a range of 75:25 to 99:1, more preferred in a ratio in a range of 80:20 to 98:2, still more preferred in ratio in a range of 90:10 to 95:5.

Likewise, for said mixtures, the ratios of first linear alkanediol:second alkanediol or ranges of ratios as described above are also applicable.

By these mixing the alkanediols in the above-specified ratios the afore-mentioned mixtures show a synergistic efficacy when used in combination with a cooling agent component in direct comparison with the corresponding individual substances as it is described and demonstrated in the experimental part.

A preferred variant according to the second aspect the present invention also encompasses a mixture including as first linear alkanediol 1,2-heptanediol and as second linear alkanediol 2,3-octanediol or a mixture including as first linear alkanediol 1,2-octanediol and as second linear alkanediol 2,3-heptanediol either in a ratio in a range of 50:50 to 99.9:0.1, preferably in a ratio in a range of 75:25 to 99:1, more preferred in a ratio in a range of 80:20 to 98:2 or still more preferred in a ratio in a range of 90:10 to 95:5.

Likewise, for said mixtures, the ratios of first linear alkanediol:second alkanediol or ranges of ratios as described above are also applicable.

A preferred variant according to the second aspect the present invention also encompasses a mixture including as first linear alkanediol 1,2-heptanediol and as second linear alkanediol 2,3-heptanediol or a mixture including as first linear alkanediol 1,2-octanediol and as second linear alkanediol 2,3-octanediol or a mixture including as first linear alkanediol 1,2-nonanediol and as second linear alkanediol 2,3-nonanediol or a mixture including as first linear alkanediol 1,2-undecanediol and as second linear alkanediol 2,3-undecanediol either in a ratio in a range of 50:50 to 99.9:0.1, preferably in a ratio in a range of 75:25 to 99:1, more preferred in a ratio in a range of 80:20 to 98:2 or still more preferred in a ratio in a range of 90:10 to 95:5.

Likewise, for said mixtures, the ratios of first linear alkanediol:second alkanediol or ranges of ratios as described above are also applicable.

The alkanediols are obtained either by synthesis from petrochemical or other fossil fuel sources by known methods such as olefin bishydroxylation, hydrolysis from epoxide or various chemical transformations or from bioderived feedstock by fermentation or from bio-based natural and renewable feedstock such as biomass by catalytic synthesis as it is described in US 2019/0241491 A1 and US 2020/0189995 A1. The alkanediols used according to the present invention comprise either petrochemically derived and biobased natural and renewable feedstock derived alkanediols. Preferably, the alkanediols are from bio-based sources and are thus bio-alkanediols.

Preferably, the homecare product composition or the cosmetic or pharmaceutical composition according to the present invention, comprises component (a) the 1,2-nonanediol and/or the 2,3-nonanediol or the at least one linear alkanediol or the mixture of at least one first linear alkanediol with one or more second linear alkanediol(s) in an amount of 0.001 to 15.0% by weight, more particularly in an amount of 0.01 to 10.0% by weight, most particularly in an amount of 0.1 to 5% by weight, based on the total weight of the composition.

In a preferred embodiment the homecare product composition or the cosmetic or pharmaceutical composition according to the present invention, comprises a mixture of at least one first linear alkanediol with one or more second linear alkanediol(s), wherein the at least one first linear alkanediol is present in an amount of 0.001 to 15.0% by weight, particularly in an amount of 0.01 to 10.0% by weight, most particularly in an amount of 0.1 to 5.0% by weight, based on the total weight of the composition; and wherein the one or more second linear alkanediol(s) is present in an amount of 0.001 to 15.0% by weight, particularly in an amount of 0.01 to 10.0% by weight, most particularly in an amount of 0.1 to 5.0% by weight, based on the total weight of the composition.

Component (b)

In the present invention the term “cooling agent” refers to a physiological cooling agent. The composition according to the present invention is particularly advantageously combined with one or more physiological cooling agent(s).

In a preferred embodiment the at least one cooling agent is a skin-cooling agent.

The use of cooling agents can for example alleviate itching. Preferred individual cooling agents for use within the framework of the present invention are listed below. The person skilled in the art can add many other cooling agents to this list; the cooling agents listed can also be used in combination with one another.

Preferably, the at least one cooling agent is referring to at least four cooling agents, more preferably the at least one cooling agent is at least three cooling agents, even more preferably the at least one cooling agent is at least two cooling agents, most preferably the at least one cooling agent is at least one cooling agent.

Preferably, the at least one cooling agent is four cooling agents, more preferably the at least one cooling agent is three cooling agents, even more preferably the at least one cooling agent is two cooling agents, most preferably the at least one cooling agent is one cooling agent.

The at least one cooling agent according to the present invention may be any physiological cooling agent known in the art.

In one embodiment the homecare product composition or the cosmetic or pharmaceutical compositions according to the present invention contains at least one cooling agent with a physiological cooling effect, which are preferably selected here from the following list: menthol and menthol derivatives (for example L-menthol, D-menthol, racemic menthol, isomenthol, neoisomenthol, neomenthol), menthylethers (for example (l-menthoxy)-1,2-propandiol, (l-menthoxy)-2-methyl-1,2-propandiol, l-menthyl-methylether), menthylesters (for example menthylformiate, menthylacetate, menthylisobutyrate, menthyhydroxyisobutyrat, menthyllactates, L-menthyl-L-lactate, L-menthyl-D-lactate, menthyl-(2-methoxy)acetate, menthyl-(2-methoxyethoxy) acetate, menthylpyroglutamate), menthylcarbonates (for example menthylpropylene glycolcarbonate, menthylethyleneglycolcarbonate, menthylglycerolcarbonate or mixtures thereof), the semi-esters of menthols with a dicarboxylic acid or derivatives thereof (for example mono-menthylsuccinate, mono-menthylglutarate, mono-menthylmalonate, O-menthyl succinic acid ester-N,N-(dimethyl)amide, O-menthyl succinic acid ester amide), menthanecarboxylic acid amides (in this case preferably menthanecarboxylic acid-N-ethylamide [WS3] or N^{α}-(menthanecarbonyl) glycinethylester [WS5], as described in U.S. Pat. No. 4,150,052, menthanecarboxylic acid-N-(4-cyanophenyl)amide or menthanecarboxylic acid-N-(4-cyanomethylphenyl)amide as described in WO 2005 049553 A1, methanecarboxylic acid-N-(alkoxyalkyl)amides), menthone and menthone derivatives (for example menthone glyceryl acetal, menthone glyceryl ketal or mixtures of both, L-menthone glycerol ketal, (−)-menthone 1,2-glycerol ketal), 2,3-dimethyl-2-(2-propyl)-butyric acid derivatives (for example 2,3-dimethyl-2-(2-propyl)-butyric acid-N-methylamide [WS23]), isopulegol or its esters (I-(−)-isopulegol, I-(−)-isopulegolacetate), menthane derivatives (for example p-menthane-3,8-diol), cubebol or synthetic or natural mixtures, containing cubebol, pyrrolidone derivatives of cycloalkyldione derivatives (for example 3-methyl-2(1-pyrrolidinyl)-2-cyclopentene-1-one) or tetrahydropyrimidine-2-one (for example iciline or related compounds, as described in WO 2004/026840), further carboxamides (for example N-(2-(pyridin-2-yl)ethyl)-3-p-menthanecarbox-amide, N-Cyclopropyl-5-methyl-2-iso-propyl cyclohexanecarboxamide, N-(4-cyanomethylphenyl)-p-menthane carboxamide, N-(4-(carbamoylmethyl)phenyl)-menthylcarboxamide, 2-(p-tolyloxy)-N-(1H-pyrazol-5-yl)-N-((thiophen-2-yl)methyl)acetamide or related compounds), (1R,2S,5R)—N-(4-Methoxyphenyl)-5-methyl-2-(1-isopropyl)cyclohexane-carboxamide [WS12], oxa-mates (preferably those described in EP 2033688 A2), [(1R,2S,5R)-2-isopropyl-5-methyl-cyclohexyl] 2-(ethylamino)-2-oxo-acetate (X Cool), Menthoxyethanol/Methyloxyethanol, (1R,2R,4R)-1-(2-Hydroxy-4-methylcyclohexyl) ethanone, 2-Isopropyl-N,2,3-trimethylbutyramide, N-Ethyl-2,2-diisopropylbutanamide, N-(1,1-Dimethyl-2-hydroxyethyl)-2,2-diethylbutanamide, N-(2-Hydroxyethyl)-2-isopropyl-2,3-dimethylbutanamide, N-(2-Hydroxyethyl)-2,3-dimethyl-2-isopropylbutanamide, Menthyl Ethylamido Oxalat, or a mixture of two or more of the afore-mentioned cooling agents.

Cooling agents which are preferred due to their particular synergistic effect are l-menthol, d-menthol, racemic menthol, menthone glycerol acetal (trade name: Frescolat® MGA), menthyl lactate (preferably l-menthyl lactate, in particular l-menthyl l-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol. Particularly preferred cooling agents are l-menthol, racemic menthol, menthone glycerol acetal (trade name: Frescolat® MGA), menthyl lactate (preferably l-menthyl lactate, in particular l-menthyl l-lactate (trade name: Frescolat® ML)), 3-menthoxypropane-1,2-diol, 2-hydroxyethyl menthyl carbonate and 2-hydroxypropyl menthyl carbonate. Very particularly preferred cooling agents are l-menthol, menthone glycerol acetal (trade name: Frescolat® MGA) and menthyl lactate (preferably l-menthyl lactate, in particular l-menthyl l-lactate (trade name: Frescolat® ML).

In a preferred embodiment, the at least one cooling agent is selected from the group consisting of menthol, menthol derivatives, cyclohexanol derivatives, cyclic secondary and tertiary alcohols, alkylmethanols, trialkylphosphine oxides, cyclic and acyclic sulfoxides, cyclic and acyclic sulfones, cyclic amides, acyclic amides, menthol-derived amides and carboxamides, more preferably selected from the group consisting of menthol, menthol derivatives, menthol-derived amides, alkylmethanols, cyclic amides and acyclic amides, even more preferably selected from the group consisting of menthol, menthol derivatives and menthol-derived amides.

In a preferred embodiment, the at least one cooling agent is selected from the group consisting of menthol and menthol derivatives (for example L-menthol, D-menthol, racemic menthol, isomenthol, neoisomenthol, neomenthol), Frescolat ML ((−)-Menthyl lactate) (FEMA 3748), N-Ethyl-p-menthane-3-carboxamide (FEMA 3455), [Ethyl 3-(p-menthane-3-carboxamido)acetate] (FEMA 4309), (1R,2S,5R)—N-(4-Methoxyphenyl)-p-menthanecarboxamide (FEMA 4681), (−)-Isopulegol (FEMA 2962), 3-((−)-Menthoxy)propane-1,2-diol (FEMA 3784), Frescolat® MGC ((−)-Menthyl ethylene glycol carbonate) (FEMA 3805), Frescolat® MGA ((−)-Menthone 1,2-glycerol ketal) (FEMA 3807), Frescolat® MGA DL-Menthone 1,2-glycerol ketal (FEMA 3808), (−)-Menthyl succinate (FEMA 3810), Frescolat® MPC ((−)-Menthyl 1&2 propylene glycol carbonates ((FEMA 3806), Frescolat® Type MPC (racemic) DL-Menthyl 1&2 propylene glycol carbonates (FEMA 3992), (−)-Menthyl glutarate (FEMA 4006), (+)-cis & (−)-trans p-menthane-3,8-diol (FEMA 4053), (−)-Menthyl pyrrolidone carboxylate (FEMA 4155), Di-(−)-menthyl glutarate (FEMA 4604), (−)-Menthone (S)-lactic acid ketal (FEMA 4285), (−)-Menthyl (S)-3-hydroxybutyrate (FEMA 4308), (−)-Menthyl acetoacetate (FEMA 4327), 2-[2-(p-menthan-3-yloxy)ethoxy]ethanol=(1R,2S,5R)-2-[2-(2-Isopropyl-5-methyl-cyclohexyloxy)ethoxy]-ethanol (FEMA 4718), (−)-Cubebol=(1R,4S,5R,6R,7S,10R)-7-isopropyl-4,10-dimethyl-tricyclo[4.4.0.0(1,5)]decan-4-ol (FEMA 4497), (1R,2R,4R)-1-(2-Hydroxy-4-methylcyclohexyl)ethanone (FEMA 4742), Menthoxyethanol/Methyloxyethanol (FEMA 4154), N-Cyclopropyl-5-methyl-2-isopropylcyclohexanecarboxamide (FEMA 4693), N-(4-cyanomethylphenyl)-p-menthanecarboxamide (FEMA 4154), N-(2-(Pyridin-2-yl)ethyl)-3-p-menthanecarboxamide (FEMA 4496), N-(4-(carbamoylmethyl)phenyl)-menthylcarboxamide (FEMA 4602), 2-(p-tolyloxy)-N-(1H-pyrazol-5-yl)-N-((thiophen-2-yl)methyl)acetamide (FEMA 4809), (2-Isopropyl-N,2,3-trimethylbutyramide) (FEMA 3804), (N-Ethyl-2,2-diisopropylbutanamide) (FEMA 4557), (N-(1,1-Dimethyl-2-hydroxyethyl)-2,2-diethylbutanamide) (FEMA 4603), N-(2-Hydroxyethyl)-2-isopropyl-2,3-dimethylbutanamide (FEMA 4549), N-(2-Hydroxyethyl)-2,3-dimethyl-2-isopropylbutanamide (FEMA 4602), (N-Ethyl-p-menthane-3-carboxamide) (FEMA 3455), [Ethyl 3-(p-menthane-3-carboxamido)acetate] (FEMA 4309), (1R,2S,5R)—N-(4-Methoxyphenyl)-p-menthanecarboxamide (FEMA 4681), (1R,2S,5R)—N-(4-(cyanomethyl)phenyl)menthylcarboxamide (FEMA 4496), N,N-Dimethyl (−)-menthyl succinamide (FEMA 4230), (1R,2S,5R)—N-(2-(pyridin-2-yl)ethyl)menthylcarboxamide (FEMA 4549), (1R,2S,5R)—N-(4-(carbamoylmethyl)phenyl)-menthylcarboxamide (FEMA 4684), N-(2-(pyridin-2-yl)ethyl)-3-p-menthanecarboxamide or related compounds, (1R,2S,5R)—N-(4-Methoxyphenyl)-5-methyl-2-(1-isopropyl)cyclohexane-carboxamide, oxamates (preferably those described in EP 2033688 A2), [(1R,2S,5R)-2-isopropyl-5-methyl-cyclohexyl] 2-(ethylamino)-2-oxo-acetate and Menthyl Ethylamido Oxalat.

In a particularly preferred embodiment the at least one cooling agent is selected from the group consisting of menthol, menthol derivatives and menthol-derived amides, and preferably wherein the at least one cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and Menthyl Ethylamido Oxalat, in a more preferred embodiment the at least one cooling agent is selected from the group consisting of (−)-menthyl lactate, 3-((−)-menthoxy)propane-1,2-diol, (−)-menthone 1,2-glycerol ketal and N-ethyl-p-menthane-3-carboxamide, in the most preferred embodiment the cooling agent is (−)-Menthyl lactate.

The homecare product composition or the cosmetic or pharmaceutical composition according to the present invention, comprises component (b) the at least one cooling agent in an amount of 0.0001 to 10.0% by weight, particularly in an amount of 0.001 to 5.0% by weight, more particularly in an amount of 0.005 to 4.0% by weight, most particularly in an amount of 0.01 to 3.0% by weight.

In a preferred embodiment, the homecare product composition or the cosmetic or pharmaceutical composition according to the present invention is a homecare product composition or a cosmetic or pharmaceutical composition, comprising or consisting of:
(a) 1,2-nonanediol and/or 2,3-nonanediol;
(b) at least one cooling agent, wherein the cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and menthyl ethylamido oxalat;
(c) optionally at least one antimicrobial compound; and
(d) optionally at least one active substance for a cosmetic or pharmaceutical composition or a homecare product composition and/or additive.

In a preferred embodiment, the homecare product composition or the cosmetic or pharmaceutical composition according to the present invention is a homecare product composition or a cosmetic or pharmaceutical composition, comprising or consisting of:
(a1) 1,2-nonanediol;
(b1) at least one cooling agent, wherein the cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and menthyl ethylamido oxalat;
(c1) optionally at least one antimicrobial compound; and
(d1) optionally at least one active substance for a cosmetic or pharmaceutical composition or a homecare product composition and/or additive.

In a preferred embodiment, the homecare product composition or the cosmetic or pharmaceutical composition according to the present invention is a homecare product composition or a cosmetic or pharmaceutical composition, comprising or consisting of:
(a2) 2,3-nonanediol;
(b2) at least one cooling agent, wherein the cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and menthyl ethylamido oxalat;
(c2) optionally at least one antimicrobial compound; and
(d2) optionally at least one active substance for a cosmetic or pharmaceutical composition or a homecare product composition and/or additive.

In a preferred embodiment, the homecare product composition or the cosmetic or pharmaceutical composition according to the present invention is a homecare product composition or a cosmetic or pharmaceutical composition, comprising or consisting of:
(a3) a mixture of 1,2-nonanediol and 2,3-nonanediol;
(b3) at least one cooling agent, wherein the cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and menthyl ethylamido oxalat;
(c3) optionally at least one antimicrobial compound; and
(d3) optionally at least one active substance for a cosmetic or pharmaceutical composition or a homecare product composition and/or additive.

In a preferred embodiment the homecare product composition or the cosmetic or pharmaceutical composition according to the present invention is a homecare product composition or a cosmetic or pharmaceutical composition, comprising or consisting of:
(a) 1,2-nonanediol and/or 2,3-nonanediol;
(b) at least one cooling agent, wherein the cooling agent is selected from the group consisting of (−)-menthyl lactate, 3-((−)-menthoxy)propane-1,2-diol, (−)-menthone 1,2-glycerol ketal and N-ethyl-p-menthane-3-carboxamide;
(c) optionally at least one antimicrobial compound; and
(d) optionally at least one active substance for a cosmetic or pharmaceutical composition or a homecare product composition and/or additive.

In a preferred embodiment the homecare product composition or the cosmetic or pharmaceutical composition according to the present invention is a homecare product composition or a cosmetic or pharmaceutical composition, comprising or consisting of:
(a) 1,2-nonanediol and/or 2,3-nonanediol;
(b) a cooling agent, wherein the cooling agent is (−)-menthyl lactate;
(c) optionally at least one antimicrobial compound; and
(d) optionally at least one active substance for a cosmetic or pharmaceutical composition or a homecare product composition and/or additive.

In a preferred embodiment the homecare product composition or the cosmetic or pharmaceutical composition according to the present invention is a homecare product composition or cosmetic or pharmaceutical composition, comprising or consisting of:
(a) at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol;
(b) at least one cooling agent, wherein the cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and menthyl ethylamido oxalat;
(c) optionally at least one antimicrobial compound; and
(d) optionally at least one active substance for a cosmetic or pharmaceutical composition or a homecare product composition and/or additive.

In a preferred embodiment the homecare product composition or the cosmetic or pharmaceutical composition according to the present invention is a homecare product composition or a cosmetic or pharmaceutical composition, comprising or consisting of:
(a) at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol;
(b) at least one cooling agent, wherein the cooling agent is selected from the group consisting of (−)-menthyl lactate, 3-((−)-menthoxy)propane-1,2-diol, (−)-menthone 1,2-glycerol ketal and N-ethyl-p-menthane-3-carboxamide;
(c) optionally at least one antimicrobial compound; and
(d) optionally at least one active substance for a cosmetic or pharmaceutical composition or a homecare product composition and/or additive.

In a preferred embodiment the homecare product composition or the cosmetic or pharmaceutical composition according to the present invention is a homecare product composition or a cosmetic or pharmaceutical composition, comprising or consisting of:
(a) at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol;
(b) a cooling agent, wherein the cooling agent is (−)-menthyl lactate;
(c) optionally at least one antimicrobial compound; and
(d) optionally at least one active substance for a cosmetic or pharmaceutical composition or a homecare product composition and/or additive.

In a preferred embodiment the homecare product composition or the cosmetic or pharmaceutical composition according to the present invention is a homecare product composition or a cosmetic or pharmaceutical composition, comprising or consisting of:
(a) at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol, wherein the at least one linear alkanediol or the at least one first linear alkanediol is selected from the group consisting of 1,2-heptanediol, 2,3-heptanediol, 1,2-nonanediol, 2,3-nonanediol, 1,2-undecanediol and 2,3-undecanediol;
(b) at least one cooling agent, wherein the cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and menthyl ethylamido oxalat;
(c) optionally at least one antimicrobial compound; and
(d) optionally at least one active substance for a cosmetic or pharmaceutical composition or a homecare product composition and/or additive.

In a preferred embodiment the homecare product composition or the cosmetic or pharmaceutical composition according to the present invention is a homecare product composition or a cosmetic or pharmaceutical composition, comprising or consisting of:
(a) at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol, wherein the at least one linear alkanediol or the at least one first linear alkanediol is selected from the group consisting of 1,2-heptanediol, 2,3-heptanediol, 1,2-nonanediol, 2,3-nonanediol, 1,2-undecanediol and 2,3-undecanediol;
(b) at least one cooling agent, wherein the cooling agent is selected from the group consisting of (−)-menthyl lactate, 3-((−)-menthoxy)propane-1,2-diol, (−)-menthone 1,2-glycerol ketal and N-ethyl-p-menthane-3-carboxamide;
(c) optionally at least one antimicrobial compound; and
(d) optionally at least one active substance for a cosmetic or pharmaceutical composition or a homecare product composition and/or additive.

In a preferred embodiment the homecare product composition or the cosmetic or pharmaceutical composition according to the present invention is a homecare product composition or a cosmetic or pharmaceutical composition, comprising or consisting of
(a) at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol, wherein the at least one linear alkanediol or the at least one first linear alkanediol is selected from the group consisting of 1,2-heptanediol, 2,3-heptanediol, 1,2-nonanediol, 2,3-nonanediol, 1,2-undecanediol and 2,3-undecanediol;
(b) a cooling agent, wherein the cooling agent is (−)-menthyl lactate;
(c) optionally at least one antimicrobial compound; and
(d) optionally at least one active substance for a cosmetic or pharmaceutical composition or a homecare product composition and/or additive.

Component (c)

The at least one antimicrobial compound according to the present invention may be any antimicrobial compound known in the art.

Preferably, the homecare product composition or the cosmetic or pharmaceutical composition according to the present invention comprises at least one antimicrobial compound.

Preferably, the at least one antimicrobial compound is at least four antimicrobial compounds, more preferably the at least one antimicrobial compound is at least three antimicrobial compounds, even more preferably the at least one antimicrobial compound is at least two antimicrobial compounds, most preferably the at least one antimicrobial compound is at least one antimicrobial compound.

Preferably, the at least one antimicrobial compound is four antimicrobial compounds, more preferably the at least one antimicrobial compound is three antimicrobial compounds, even more preferably the at least one antimicrobial compound is two antimicrobial compounds, most preferably the at least one antimicrobial compound is one antimicrobial compound.

The term “antimicrobial component” in the context of the present invention refers to a group of substances, in particular to an agent that kills microorganisms or stops or inhibits their growth. Antimicrobial agents can be grouped according to the microorganisms they act primarily against. For example, antibiotics are used against bacteria, antifungals are used against fungi, antiprotozoals are used against protozoans, and antivirals are used against virus. They can also be classified according to their function. Agents that kill microbes are biocidal or microbicidal, while those that merely inhibit their growth are called biostatic.

The antimicrobial component according to the present invention act primarily against microorganisms, in particular bacteria, yeast and/or fungi. Specified microorganisms are mesophilic bacteria or yeast or fungi which have relevance for the production of compounds having an unpleasant smell from human sweat. Microorganisms considered as specified microorganisms according to the present invention are preferably of the genus Pseudomonas (bacterium), Staphylococcus (bacterium), Escherichia (bacterium), Candida (yeast) and Aspergillus (fungi), and combinations thereof.

More preferably, the microorganisms are selected from the group consisting of Pseudomonas aeruginosa, Staphylococcus aureus, Escherichia coli, Candida albicans and Aspergillus brasiliensis, and combinations thereof.

Suitable antimicrobial agents are, in principle, all substances effective against bacteria, such as, for example, 4-hydroxybenzoic acid and its salts and esters, N-(4-chlorophenyl)-N′-(3,4-dichlorophenyl)urea, 2,4,4′-trichloro-2′-hydroxy-diphenyl ether (triclosan), 4-chloro-3,5-dimethyl-phenol, 2,2′-methylenebis(6-bromo-4-chlorophenol), 3-methyl-4-(1-methylethyl)phenol, 2-benzyl-4-chloro-phenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, oil of cloves, mint oil, farnesol, phenoxyethanol, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide.

In a preferred embodiment, the at least one antimicrobial compound is selected from the group consisting of Phenoxyethanol, Farnesol, bisabolol, Ethylhexylglycerin, Triethyl citrate, Hydroxyacetophenone, Benzyl Alcohol, 2-Methyl 5-cyclohexylpentanol, Benzoic Acid, Sorbic Acid, Dehydroacetic Acid, Sorbitan Caprylate, Caprylhydroxamic Acid, Dimethyl phenylbutanol, Dimethyl phenylpropanol, Levulinic Acid and esters thereof, Anisic Acid, 2-benzylheptanol, Polyglyceryl-3 caprylate, Polyglyceryl-10 Laurate, glyceryl caprate, polyglyceryl-2 caprate, Zinc ricinoleate, Glyceryl laurate, glyceryl caprylate, Xylityl Sesquicaprylate, phenylpropanol, lauryl alcohol, o-cymen-5-ol, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, tropolone, piroctone olamine, triclosan, triclocarban, chloroxylenol, chlorocresol, benzalkonium chloride, benzalkonium bromide, benzalkonium saccharinate, benzethonium chloride, chlorphenesin, alkyl (C 12-22) trimethyl ammonium bromide and chloride, zinc pyrithione, climbazole, chlorhexidine, chlorhexidine diacetate, chlorhexidine digluconate, chlorhexidine dihydrochloride, hexamidine, hexamidine diisethionate, decylene glycol, silver chloride, silver citrate, Hexylglycerin, Cyclohexylglycerin, glycerin, Tetraselmis extract, mannitol, sorbitol, lactic acid, sodium lauroyl lactylate, sodium caproyl lactylate, salicylic acid and its salts, capryloyl glycine, ethyl lauroyl arginate HCl, Thymol, jasmol, zinc citrate, cetylpyridinium chloride, menthol, eucalyptol, methyl salicylate, ethanol, camphor, polyarginine, polylysine, gluconic acid and salts thereof, ascorbic acid and salts therof, benzyl salicylate, Disodium EDTA, Methylparaben and its salts, Sodium Benzoate, Potassium Sorbate, Propylparaben, Methylchloroisothiazolinone, Methylisothiazolinone, Butylparaben, Ethylparaben, Sodium Propylparaben, DMDM Hydantoin, Hydroxyethoxyphenyl Butanone, Hydroxyethoxyphenyl Butanol, Itaconic Acid and salts thereof, Propanediol Caprylate, 4-Hydroxyacetophenone, Iodopropynyl Butylcarbamate, Isobutylparaben, Sodium Ethylparaben, Diazolidinyl Urea, Glyceryl Caprylate, Sodium Phytate or Phytic acid, Sodium Levulinate or Levulinic Acid and esters thereof, TEA-Salicylate, Phenethyl Alcohol, Tetrasodium Glutamate Diacetate, Trisodium Ethylenediamine Disuccinate, Sodium Lactate.

Further preferred, the at least one antimicrobial compound is selected from the group consisting of Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 2,3-pentanediol, 2,3-hexanediol, 2,3-heptanediol, 2,3-octanediol, 2,3-nonanediol, 1,2-decanediol.

In a more preferred embodiment, the at least one antimicrobial compound is selected from the group consisting of Phenoxyethanol, 2-Methyl 5-cyclohexylpentanol, Dimethyl phenylbutanol, Dimethyl phenylpropanol, 2-benzylheptanol, Ethylhexylglycerin, Hydroxyacetophenone, Triethyl citrate, Polyglyceryl-3 caprylate, Polyglyceryl-10 Laurate, glyceryl caprylate, Benzyl Alcohol, Benzoic Acid, Sorbic Acid, Levulinic Acid and esters thereof, Anisic Acid and salts thereof, polyglyceryl-2 caprate, Caprylhydroxamic Acid, Zinc ricinoleate, Farnesol, bisabolol, Glyceryl laurate, glyceryl caprylate, Xylityl Sesquicaprylate, phenylpropanol, lauryl alcohol, o-cymen-5-ol, tropolone, climbazole, piroctone olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis extract, Mannitol, sorbitol, lactic acid, sodium lauroyl lactylate, sodium caproyl lactylate, salicylic acid and its salts, capryloyl glycine, ethyl lauroyl arginate HCl, Thymol, jasmol, gluconic acid and salts thereof, benzyl salicylate, Propanediol Caprylate, 4-Hydroxyacetophenone.

In an even more preferred embodiment, the at least one antimicrobial compound is selected from the group consisting of 2-Methyl 5-cyclohexylpentanol, Dimethyl phenylbutanol, Ethylhexylglycerin, Triethyl citrate, Polyglyceryl-3 caprylate, o-Cymen-5-ol, piroctone olamine, farnesol, Xylityl Sesquicaprylate, glyceryl caprylate, Phenoxyethanol, Farnesol, climbazole, Triethyl citrate, Benzyl Alcohol, o-cymen-5-ol, salicylic acid and its salts, benzyl salicylate, Propanediol Caprylate.

In a still even more preferred embodiment, the at least one antimicrobial compound is selected from the group consisting of Ethylhexylglycerin, Triethyl citrate, Polyglyceryl-3 caprylate, farnesol, 2-Methyl 5-cyclohexylpentanol (SymDeo B125).

The homecare product composition or the cosmetic or pharmaceutical composition according to the present invention, comprises component (c) the at least one antimicrobial compound in an amount of 0.001 to 15.0% by weight, particularly in an amount of 0.005 to 10.0% by weight, most particularly in an amount of 0.01 to 5% by weight, based on the total weight of the composition.

In a particularly preferred embodiment, the homecare product composition or the cosmetic or pharmaceutical composition of the present invention is a homecare product composition or a cosmetic or pharmaceutical composition, wherein the at least one linear alkanediol or the at least one first linear alkanediol or the one or more second linear alkanediol(s) are selected from the group consisting of 1,2-heptanediol, 2,3-heptanediol, 1,2-nonanediol, 2,3-nonanediol, 1,2-undecanediol and 2,3-undecanediol; wherein the at least one cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and Menthyl ethylamido oxalat; and

wherein the composition comprises an antimicrobial compound which is selected from the group consisting of ethylhexylglycerin, triethyl citrate, polyglyceryl-3 caprylate, farnesol and 2-methyl 5-cyclohexylpentanol.

In a particularly preferred embodiment, the homecare product composition or the cosmetic or pharmaceutical composition of the present invention is a homecare product composition or a cosmetic or pharmaceutical composition, wherein the at least one linear alkanediol or the at least one first linear alkanediol or the one or more second linear alkanediol(s) are selected from the group consisting of 1,2-heptanediol, 2,3-heptanediol, 1,2-nonanediol, 2,3-nonanediol, 1,2-undecanediol and 2,3-undecanediol; wherein the at least one cooling agent is selected from the group consisting of (−)-menthyl lactate, 3-((−)-menthoxy)propane-1,2-diol, (−)-menthone 1,2-glycerol ketal and N-ethyl-p-menthane-3-carboxamide; and

wherein the composition comprises an antimicrobial compound which is selected from the group consisting of ethylhexylglycerin, triethyl citrate, polyglyceryl-3 caprylate, farnesol and 2-methyl 5-cyclohexylpentanol.

In a particularly preferred embodiment, the homecare product composition or the cosmetic or pharmaceutical composition of the present invention is a homecare product composition or a cosmetic or pharmaceutical composition, wherein the at least one linear alkanediol or the at least one first linear alkanediol or the one or more second linear alkanediol(s) are selected from the group consisting of 1,2-heptanediol, 2,3-heptanediol, 1,2-nonanediol, 2,3-nonanediol, 1,2-undecanediol and 2,3-undecanediol; wherein the at least one cooling agent is (−)-menthyl lactate; and wherein the composition comprises an antimicrobial compound which is selected from the group consisting of ethylhexylglycerin, triethyl citrate, polyglyceryl-3 caprylate, farnesol and 2-methyl 5-cyclohexylpentanol.

In a particularly preferred embodiment, the homecare product composition or the cosmetic or pharmaceutical composition of the present invention is a homecare product composition or a cosmetic or pharmaceutical composition, wherein the at least one linear alkanediol, the at least one first linear alkanediol and/or the one or more second linear alkanediol(s) are selected from the group consisting of 1,2-heptanediol, 2,3-heptanediol, 1,2-nonanediol, 2,3-nonanediol, 1,2-undecanediol and 2,3-undecanediol;

wherein the at least one cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and menthyl ethylamido oxalat; and
wherein the composition comprises an antimicrobial compound which is selected from the group consisting of ethylhexylglycerin, triethyl citrate, polyglyceryl-3 caprylate, farnesol and 2-methyl 5-cyclohexylpentanol.

In a particularly preferred embodiment, the homecare product composition or the cosmetic or pharmaceutical composition of the present invention is a homecare product composition or a cosmetic or pharmaceutical composition, wherein the at least one linear alkanediol, the at least one first linear alkanediol and/or the one or more second linear alkanediol(s) are selected from the group consisting of 1,2-heptanediol, 2,3-heptanediol, 1,2-nonanediol, 2,3-nonanediol, 1,2-undecanediol and 2,3-undecanediol;

wherein the at least one cooling agent is selected from the group consisting of (−)-menthyl lactate, 3-((−)-menthoxy)propane-1,2-diol, (−)-menthone 1,2-glycerol ketal and N-ethyl-p-menthane-3-carboxamide; and
wherein the composition comprises an antimicrobial compound which is selected from the group consisting of ethylhexylglycerin, triethyl citrate, polyglyceryl-3 caprylate, farnesol and 2-methyl 5-cyclohexylpentanol.

In a particularly preferred embodiment, the homecare product composition or the cosmetic or pharmaceutical composition of the present invention is a homecare product composition or a cosmetic or pharmaceutical composition, wherein the at least one linear alkanediol, the at least one first linear alkanediol and/or the one or more second linear alkanediol(s) are selected from the group consisting of 1,2-heptanediol, 2,3-heptanediol, 1,2-nonanediol, 2,3-nonanediol, 1,2-undecanediol and 2,3-undecanediol;

wherein the at least one cooling agent is (−)-menthyl lactate; and
wherein the composition comprises an antimicrobial compound which is selected from the group consisting of ethylhexylglycerin, triethyl citrate, polyglyceryl-3 caprylate, farnesol and 2-methyl 5-cyclohexylpentanol.

In a particularly preferred embodiment, the homecare product composition or the cosmetic or pharmaceutical composition of the present invention is a homecare product composition or a cosmetic or pharmaceutical composition comprising or consisting of the following combinations of components (a), (b) and (c):
1,2-heptanediol, (−)-menthyl lactate, ethylhexylglycerin;
or 1,2-heptanediol, (−)-menthyl lactate, triethyl citrate;
or 1,2-heptanediol, (−)-menthyl lactate, polyglyceryl-3 caprylate;
or 1,2-heptanediol, (−)-menthyl lactate, farnesol;
or 1,2-heptanediol, (−)-menthyl lactate, 2-methyl 5-cyclohexylpentanol;
or 1,2-heptanediol, menthol, ethylhexylglycerin;
or 1,2-heptanediol, menthol, triethyl citrate;
or 1,2-heptanediol, menthol, polyglyceryl-3 caprylate;
or 1,2-heptanediol, menthol, farnesol;
or 1,2-heptanediol, menthol, 2-methyl 5-cyclohexylpentanol;
or 1,2-heptanediol, (−)-menthone 1,2-glycerol ketal, ethylhexylglycerin;
or 1,2-heptanediol, (−)-menthone 1,2-glycerol ketal, triethyl citrate;
or 1,2-heptanediol, (−)-menthone 1,2-glycerol ketal, polyglyceryl-3 caprylate;
or 1,2-heptanediol, (−)-menthone 1,2-glycerol ketal, farnesol;
or 1,2-heptanediol, (−)-menthone 1,2-glycerol ketal, 2-methyl 5-cyclohexylpentanol;
or 1,2-heptanediol, 3-((−)-menthoxy)propane-1,2-diol, ethylhexylglycerin;
or 1,2-heptanediol, 3-((−)-menthoxy)propane-1,2-diol, triethyl citrate;
or 1,2-heptanediol, 3-((−)-menthoxy)propane-1,2-diol, polyglyceryl-3 caprylate;
or 1,2-heptanediol, 3-((−)-menthoxy)propane-1,2-diol, farnesol;
or 1,2-heptanediol, 3-((−)-menthoxy)propane-1,2-diol, 2-methyl 5-cyclohexylpentanol;
or 1,2-heptanediol, N-ethyl-p-menthane-3-carboxamide, ethylhexylglycerin;
or 1,2-heptanediol, N-ethyl-p-menthane-3-carboxamide, triethyl citrate;
or 1,2-heptanediol, N-ethyl-p-menthane-3-carboxamide, polyglyceryl-3 caprylate;
or 1,2-heptanediol, N-ethyl-p-menthane-3-carboxamide, farnesol;
or 1,2-heptanediol, N-ethyl-p-menthane-3-carboxamide, 2-methyl 5-cyclohexylpentan-ol;
or 2,3-heptanediol, (−)-menthyl lactate, ethylhexylglycerin;
or 2,3-heptanediol, (−)-menthyl lactate, triethyl citrate;
or 2,3-heptanediol, (−)-menthyl lactate, polyglyceryl-3 caprylate;
or 2,3-heptanediol, (−)-menthyl lactate, farnesol;
or 2,3-heptanediol, (−)-menthyl lactate, 2-methyl 5-cyclohexylpentanol;
or 2,3-heptanediol, menthol, ethylhexylglycerin;
or 2,3-heptanediol, menthol, triethyl citrate;
or 2,3-heptanediol, menthol, polyglyceryl-3 caprylate;
or 2,3-heptanediol, menthol, farnesol;
or 2,3-heptanediol, menthol, 2-methyl 5-cyclohexylpentanol;
or 2,3-heptanediol, (−)-menthone 1,2-glycerol ketal, ethylhexylglycerin;
or 2,3-heptanediol, (−)-menthone 1,2-glycerol ketal, triethyl citrate;
or 2,3-heptanediol, (−)-menthone 1,2-glycerol ketal, polyglyceryl-3 caprylate;
or 2,3-heptanediol, (−)-menthone 1,2-glycerol ketal, farnesol;
or 2,3-heptanediol, (−)-menthone 1,2-glycerol ketal, 2-methyl 5-cyclohexylpentanol;
or 2,3-heptanediol, 3-((−)-menthoxy)propane-1,2-diol, ethylhexylglycerin;
or 2,3-heptanediol, 3-((−)-menthoxy)propane-1,2-diol, triethyl citrate;
or 2,3-heptanediol, 3-((−)-menthoxy)propane-1,2-diol, polyglyceryl-3 caprylate;
or 2,3-heptanediol, 3-((−)-menthoxy)propane-1,2-diol, farnesol;
or 2,3-heptanediol, 3-((−)-menthoxy)propane-1,2-diol, 2-methyl 5-cyclohexylpentanol;
or 2,3-heptanediol, N-ethyl-p-menthane-3-carboxamide, ethylhexylglycerin;
or 2,3-heptanediol, N-ethyl-p-menthane-3-carboxamide, triethyl citrate;
or 2,3-heptanediol, N-ethyl-p-menthane-3-carboxamide, polyglyceryl-3 caprylate;
or 2,3-heptanediol, N-ethyl-p-menthane-3-carboxamide, farnesol;
or 2,3-heptanediol, N-ethyl-p-menthane-3-carboxamide, 2-methyl 5-cyclohexylpentan-ol;
or 1,2-nonanediol, (−)-menthyl lactate, ethylhexylglycerin;
or 1,2-nonanediol, (−)-menthyl lactate, triethyl citrate;
or 1,2-nonanediol, (−)-menthyl lactate, polyglyceryl-3 caprylate;
or 1,2-nonanediol, (−)-menthyl lactate, farnesol;
or 1,2-nonanediol, (−)-menthyl lactate, 2-methyl 5-cyclohexylpentanol;
or 1,2-nonanediol, menthol, ethylhexylglycerin;
or 1,2-nonanediol, menthol, triethyl citrate;
or 1,2-nonanediol, menthol, polyglyceryl-3 caprylate;
or 1,2-nonanediol, menthol, farnesol;
or 1,2-nonanediol, menthol, 2-methyl 5-cyclohexylpentanol;
or 1,2-nonanediol, (−)-menthone 1,2-glycerol ketal, ethylhexylglycerin;
or 1,2-nonanediol, (−)-menthone 1,2-glycerol ketal, triethyl citrate;
or 1,2-nonanediol, (−)-menthone 1,2-glycerol ketal, polyglyceryl-3 caprylate;
or 1,2-nonanediol, (−)-menthone 1,2-glycerol ketal, farnesol;
or 1,2-nonanediol, (−)-menthone 1,2-glycerol ketal, 2-methyl 5-cyclohexylpentanol;
or 1,2-nonanediol, 3-((−)-menthoxy)propane-1,2-diol, ethylhexylglycerin;
or 1,2-nonanediol, 3-((−)-menthoxy)propane-1,2-diol, triethyl citrate;
or 1,2-nonanediol, 3-((−)-menthoxy)propane-1,2-diol, polyglyceryl-3 caprylate;
or 1,2-nonanediol, 3-((−)-menthoxy)propane-1,2-diol, farnesol;
or 1,2-nonanediol, 3-((−)-menthoxy)propane-1,2-diol, 2-methyl 5-cyclohexylpentanol;
or 1,2-nonanediol, N-ethyl-p-menthane-3-carboxamide, ethylhexylglycerin;
or 1,2-nonanediol, N-ethyl-p-menthane-3-carboxamide, triethyl citrate;
or 1,2-nonanediol, N-ethyl-p-menthane-3-carboxamide, polyglyceryl-3 caprylate;
or 1,2-nonanediol, N-ethyl-p-menthane-3-carboxamide, farnesol;
or 1,2-nonanediol, N-ethyl-p-menthane-3-carboxamide, 2-methyl 5-cyclohexylpentan-ol;
or 2,3-nonanediol, (−)-menthyl lactate, ethylhexylglycerin;
or 2,3-nonanediol, (−)-menthyl lactate, triethyl citrate;
or 2,3-nonanediol, (−)-menthyl lactate, polyglyceryl-3 caprylate;
or 2,3-nonanediol, (−)-menthyl lactate, farnesol;
or 2,3-nonanediol, (−)-menthyl lactate, 2-methyl 5-cyclohexylpentanol;
or 2,3-nonanediol, menthol, ethylhexylglycerin;
or 2,3-nonanediol, menthol, triethyl citrate;
or 2,3-nonanediol, menthol, polyglyceryl-3 caprylate;
or 2,3-nonanediol, menthol, farnesol;
or 2,3-nonanediol, menthol, 2-methyl 5-cyclohexylpentanol;
or 2,3-nonanediol, (−)-menthone 1,2-glycerol ketal, ethylhexylglycerin;
or 2,3-nonanediol, (−)-menthone 1,2-glycerol ketal, triethyl citrate;
or 2,3-nonanediol, (−)-menthone 1,2-glycerol ketal, polyglyceryl-3 caprylate;
or 2,3-nonanediol, (−)-menthone 1,2-glycerol ketal, farnesol;
or 2,3-nonanediol, (−)-menthone 1,2-glycerol ketal, 2-methyl 5-cyclohexylpentanol;
or 2,3-nonanediol, 3-((−)-menthoxy)propane-1,2-diol, ethylhexylglycerin;
or 2,3-nonanediol, 3-((−)-menthoxy)propane-1,2-diol, triethyl citrate;
or 2,3-nonanediol, 3-((−)-menthoxy)propane-1,2-diol, polyglyceryl-3 caprylate;
or 2,3-nonanediol, 3-((−)-menthoxy)propane-1,2-diol, farnesol;
or 2,3-nonanediol, 3-((−)-menthoxy)propane-1,2-diol, 2-methyl 5-cyclohexylpentanol;
or 2,3-nonanediol, N-ethyl-p-menthane-3-carboxamide, ethylhexylglycerin;
or 2,3-nonanediol, N-ethyl-p-menthane-3-carboxamide, triethyl citrate;
or 2,3-nonanediol, N-ethyl-p-menthane-3-carboxamide, polyglyceryl-3 caprylate;
or 2,3-nonanediol, N-ethyl-p-menthane-3-carboxamide, farnesol;
or 2,3-nonanediol, N-ethyl-p-menthane-3-carboxamide, 2-methyl 5-cyclohexylpentan-ol;
or 1,2-undecanediol, (−)-menthyl lactate, ethylhexylglycerin;
or 1,2-undecanediol, (−)-menthyl lactate, triethyl citrate;
or 1,2-undecanediol, (−)-menthyl lactate, polyglyceryl-3 caprylate;
or 1,2-undecanediol, (−)-menthyl lactate, farnesol;
or 1,2-undecanediol, (−)-menthyl lactate, 2-methyl 5-cyclohexylpentanol;
or 1,2-undecanediol, menthol, ethylhexylglycerin;
or 1,2-undecanediol, menthol, triethyl citrate;
or 1,2-undecanediol, menthol, polyglyceryl-3 caprylate;
or 1,2-undecanediol, menthol, farnesol;
or 1,2-undecanediol, menthol, 2-methyl 5-cyclohexylpentanol;
or 1,2-undecanediol, (−)-menthone 1,2-glycerol ketal, ethylhexylglycerin;
or 1,2-undecanediol, (−)-menthone 1,2-glycerol ketal, triethyl citrate;
or 1,2-undecanediol, (−)-menthone 1,2-glycerol ketal, polyglyceryl-3 caprylate;
or 1,2-undecanediol, (−)-menthone 1,2-glycerol ketal, farnesol;
or 1,2-undecanediol, (−)-menthone 1,2-glycerol ketal, 2-methyl 5-cyclohexylpentanol;
or 1,2-undecanediol, 3-((−)-menthoxy)propane-1,2-diol, ethylhexylglycerin;
or 1,2-undecanediol, 3-((−)-menthoxy)propane-1,2-diol, triethyl citrate;
or 1,2-undecanediol, 3-((−)-menthoxy)propane-1,2-diol, polyglyceryl-3 caprylate;
or 1,2-undecanediol, 3-((−)-menthoxy)propane-1,2-diol, farnesol;
or 1,2-undecanediol, 3-((−)-menthoxy)propane-1,2-diol, 2-methyl 5-cyclohexylpentan-ol;
or 1,2-undecanediol, N-ethyl-p-menthane-3-carboxamide, ethylhexylglycerin;
or 1,2-undecanediol, N-ethyl-p-menthane-3-carboxamide, triethyl citrate;
or 1,2-undecanediol, N-ethyl-p-menthane-3-carboxamide, polyglyceryl-3 caprylate;
or 1,2-undecanediol, N-ethyl-p-menthane-3-carboxamide, farnesol;
or 1,2-undecanediol, N-ethyl-p-menthane-3-carboxamide, 2-methyl 5-cyclohexyl-pentanol;
or 2,3-undecanediol, (−)-menthyl lactate, ethylhexylglycerin;
or 2,3-undecanediol, (−)-menthyl lactate, triethyl citrate;
or 2,3-undecanediol, (−)-menthyl lactate, polyglyceryl-3 caprylate;
or 2,3-undecanediol, (−)-menthyl lactate, farnesol;
or 2,3-undecanediol, (−)-menthyl lactate, 2-methyl 5-cyclohexylpentanol;
or 2,3-undecanediol, menthol, ethylhexylglycerin;
or 2,3-undecanediol, menthol, triethyl citrate;
or 2,3-undecanediol, menthol, polyglyceryl-3 caprylate;
or 2,3-undecanediol, menthol, farnesol;
or 2,3-undecanediol, menthol, 2-methyl 5-cyclohexylpentanol;
or 2,3-undecanediol, (−)-menthone 1,2-glycerol ketal, ethylhexylglycerin;
or 2,3-undecanediol, (−)-menthone 1,2-glycerol ketal, triethyl citrate;
or 2,3-undecanediol, (−)-menthone 1,2-glycerol ketal, polyglyceryl-3 caprylate;
or 2,3-undecanediol, (−)-menthone 1,2-glycerol ketal, farnesol;
or 2,3-undecanediol, (−)-menthone 1,2-glycerol ketal, 2-methyl 5-cyclohexylpentanol;
or 2,3-undecanediol, 3-((−)-menthoxy)propane-1,2-diol, ethylhexylglycerin;
or 2,3-undecanediol, 3-((−)-menthoxy)propane-1,2-diol, triethyl citrate;
or 2,3-undecanediol, 3-((−)-menthoxy)propane-1,2-diol, polyglyceryl-3 caprylate;
or 2,3-undecanediol, 3-((−)-menthoxy)propane-1,2-diol, farnesol;
or 2,3-undecanediol, 3-((−)-menthoxy)propane-1,2-diol, 2-methyl 5-cyclohexyl-pentanol;
or 2,3-undecanediol, N-ethyl-p-menthane-3-carboxamide, ethylhexylglycerin;
or 2,3-undecanediol, N-ethyl-p-menthane-3-carboxamide, triethyl citrate;
or 2,3-undecanediol, N-ethyl-p-menthane-3-carboxamide, polyglyceryl-3 caprylate;
or 2,3-undecanediol, N-ethyl-p-menthane-3-carboxamide, farnesol;
or 2,3-undecanediol, N-ethyl-p-menthane-3-carboxamide, 2-methyl 5-cyclohexyl-pentanol;

Component (d)

Within the context of the present invention, it is also possible—and in some cases advantageous—to combine the homecare product composition or the cosmetic or pharmaceutical composition according to the present invention with other active substances, adjuvants or additives.

The at least one active substance for a cosmetic or pharmaceutical composition or a homecare product composition and/or additive according to the present invention may be any cosmetically or pharmaceutically active substance or any active substance used in homecare product formulations or compositions and/or additive known in the art.

Preferably, the homecare product composition or the cosmetic or pharmaceutical composition according to the present invention comprises at least one cosmetically or pharmaceutically active substance or any active substance used in homecare product formulations or compositions and/or additive.

Optionally, other conventional active substances, adjuvants or additives, customarily used for cosmetic or pharmaceutical compositions or homecare products, as further described below, may be added as component (d), i.e. in order to obtain a ready-for-use composition or formulation.

The homecare product composition or the cosmetic or pharmaceutical composition according to the present invention can advantageously be combined with other active agents used in cosmetic or pharmaceutical compositions, preparations for animal care, or homecare product compositions and/or adjuvants and/or additives or auxiliaries, such as are customarily used in such compositions, such as for example abrasives, sebum reducing agents/anti-acne agents, agents against ageing of the skin, anti-cellulitis agents, anti-dandruff agents, anti-inflammatory agents, irritation-preventing agents, irritation-inhibiting agents, antioxidants, astringents, odour absorbers, perspiration-inhibiting agents, antiseptic agents, anti-statics, binders, buffers, carrier materials, chelating agents, cell stimulants, cleansing agents, depilatory agents, surface-active substances, deodorizing agents, antiperspirants, softeners, emulsifiers, enzymes, enzyme inhibitors, essential oils, fibres, film-forming agents, fixatives, foam-forming agents, foam stabilizers, substances for preventing foaming, foam boosters, gelling agents, gel-forming agents, hair care agents, hair-setting agents, hair-straightening agents, moisture-donating agents, moisturizing substances, moisture-retaining substances, bleaching agents, strengthening agents, stain-removing agents, optically brightening agents, impregnating agents, dirt-repellent agents, dyes, friction-reducing agents, lubricants, moisturizing creams, ointments, opacifying agents, plasticizing agents, covering agents, polish, preservatives, gloss agents, green and synthetic polymers, powders, proteins, re-oiling agents, abrading agents, silicones, skin-soothing agents, skin-cleansing agents, skin care agents, skin-healing agents, skin-lightening agents, skin-protecting agents, skin-softening agents, hair promotion agents, stabilizers, surfactants, UV-absorbing agents, UV filters, primary sun protection factors, secondary sun protection factors, detergents, fabric conditioning agents, suspending agents, skin-tanning agents, actives modulating skin or hair pigmentation, matrix-metalloproteinase inhibitors, skin moisturizing agents, glycosaminoglycan stimulators, TRPV1 antagonists, desquamating agents, anti-cellulite agents or fat enhancing agents, hair growth activators or inhibitors, thickeners, rheology additives, vitamins, oils, waxes, pearlizing waxes, fats, phospholipids, saturated fatty acids, mono- or polyunsaturated fatty acids, α-hydroxy acids, polyhydroxyfatty acids, liquefiers, dyestuffs, colour-protecting agents, pigments, anti-corrosives, fragrances or perfume oils, aromas, flavouring substances, odoriferous substances, polyols, electrolytes, organic solvents, and mixtures of two or more of the afore-mentioned substances, as further described below.

From the above active agents and/or adjuvants and/or additives or auxiliaries agents against ageing of the skin, anti-microbial agents, antioxidants, chelating agents, emulsifiers, preservatives, green and synthetic polymers, rheology additives, oils, fragrances or perfume oils, and polyols, and mixtures of two or more of the afore-mentioned substances are particularly preferred in the preparation of the homecare product composition or the cosmetic and pharmaceutical composition according to the present invention.

The cosmetically or pharmaceutically active agents or active agents used in homecare product compositions, respectively, and/or adjuvants and/or additives can in some instances provide one or more than one benefit or operate via more than one mode of action.

Since dermatological conditions or diseases are often associated with dry skin, scratched skin, skin lesions or even inflammation, the cosmetic or pharmaceutical composition according to the present invention advantageously contains preferably anti-inflammatories, antibacterial or antimycotic substances, substances having a reddening-alleviating or itch-alleviating action, lenitive substances, moisturisers and/or osmolytes, keratolytic substances, nurturing substances, anti-inflammatory, antibacterial or antimycotic substances, substances having a reddening-alleviating or itch-alleviating action, lenitive substances, antidandruff substances, or other active compounds such as solvents, fragrances antioxidants, preservatives, (metal) chelating agents, penetration enhancers, or mixtures of two or more of afore-specified agents, as further described below.

Anti-ageing actives: The cosmetic or pharmaceutical composition according to the present invention preferably contains one or more anti-ageing actives. In the context of the invention, anti-ageing or biogenic agents are, for example antioxidants, matrix-metalloproteinase inhibitors (MMPI), skin moisturizing agents, glycosaminglycan stimulators, anti-inflammatory agents, TRPV1 antagonists and plant extracts.

Antioxidants: A preferred homecare product composition or a cosmetic or pharmaceutical composition according to the present invention comprises one or more antioxidants. Suitable antioxidants encompass amino acids (preferably glycine, histidine, tyrosine, tryptophan) and derivatives thereof, imidazoles (preferably urocanic acid) and derivatives thereof, peptides, preferably D,L-carnosine, D-carnosine, L-carnosine and derivatives thereof (preferably anserine), carnitine, creatine, matrikine peptides (preferably lysyl-threonyl-threonyl-lysyl-serine) and palm itoylated pentapeptides, carotenoids, carotenes (preferably alpha-carotene, beta-carotene, lycopene) and derivatives thereof, lipoic acid and derivatives thereof (preferably dihydrolipoic acid), aurothioglucose, propyl thiouracil and other thiols (preferably thioredoxine, glutathione, cysteine, cystine, cystamine and glycosyl, N-acetyl, methyl, ethyl, propyl, amyl, butyl and lauryl, palmitoyl, oleyl, gamma-linoleyl, cholesteryl, glyceryl and oligoglyceryl esters thereof) and salts thereof, dilauryl thiodipropionate, distearyl thiodipropionate, thiodipropionic acid and derivatives thereof (preferably esters, ethers, peptides, lipids, nucleotides, nucleosides and salts) and sulfoximine compounds (preferably buthionine sulfoximines, homocysteine sulfoximine, buthionine sulfones, penta-, hexa-, heptathionine sulfoximine) in very small tolerated doses (e.g. pmol to μmol/kg), also (metal) chelators (preferably alpha-hydroxy fatty acids, palmitic acid, phytic acid, lactoferrin, alpha-hydroxy acids (preferably citric acid, lactic acid, malic acid), humic acid, bile acid, bile extracts, tannins, bilirubin, biliverdin, EDTA, EGTA and derivatives thereof), unsaturated fatty acids and derivatives thereof (preferably gamma-linolenic acid, linoleic acid, oleic acid), folic acid and derivatives thereof, ubiquinone and derivatives thereof, ubiquinol and derivatives thereof, vitamin C and derivatives (preferably ascorbyl palmitate, Mg ascorbyl phosphate, ascorbyl acetate, ascorbyl glucoside), tocopherols and derivatives (preferably vitamin E acetate), vitamin A and derivatives (vitamin A palmitate) and coniferyl benzoate of benzoic resin, rutinic acid and derivatives thereof, flavonoids and glycosylated precursors thereof, in particular quercetin and derivatives thereof, preferably alpha-glucosyl rutin, rosmarinic acid, carnosol, carnosolic acid, resveratrol, caffeic acid and derivatives thereof, sinapic acid and derivatives thereof, ferulic acid and derivatives thereof, curcuminoids, chlorogenic acid and derivatives thereof, retinoids, preferably retinyl palmitate, retinol or tretinoin, ursolic acid, Levulinic Acid and esters thereof, butyl hydroxytoluene, butyl hydroxyanisole, nordihydroguaiac acid, nordihydroguaiaretic acid, trihydroxybutyrophenone, uric acid and derivatives thereof, mannose and derivatives thereof, zinc and derivatives thereof (preferably ZnO, ZnSO₄), selenium and derivatives thereof (preferably selenium methionine), superoxide dismutase, stilbenes and derivatives thereof (preferably stilbene oxide, trans-stilbene oxide) and the derivatives (salts, esters, ethers, sugars, nucleotides, nucleosides, peptides and lipids) of these cited active ingredients which are suitable according to the invention or extracts or fractions of plants having an antioxidant effect, preferably green tea, rooibos, honeybush, grape, rosemary, sage, melissa, thyme, lavender, olive, oats, cocoa, ginkgo, ginseng, liquorice, honeysuckle, sophora, pueraria, pinus, citrus, Phyllanthus emblica or St. John's wort, grape seeds, wheat germ, Phyllanthus emblica, coenzymes, preferably coenzyme Q10, plastoquinone and menaquinone. Preferred antioxidants are selected from the group consisting of vitamin A and derivatives, vitamin C and derivatives, tocopherol and derivatives, preferably tocopheryl acetate, and ubiquinone. If vitamin E and/or derivatives thereof are used as the antioxidant(s), it is advantageous to choose their concentrations from the range from about 0.001 to about 10% by weight, based on the total weight of the composition. If vitamin A or vitamin A derivatives or carotenes or derivatives thereof are used as the antioxidant(s), it is advantageous to choose their concentrations from the range from about 0.001 to about 10% by weight based on the total weight of the composition.

Sebum reducing agents/Anti-acne agents: A preferred cosmetic or pharmaceutical composition according to the present invention comprises one or more sebum reducing agents/anti-acne agents. Suitable reducing agents/anti-acne agents encompass capryloyl glycine, propanediol caprylate, undecylenoyl glycine, farnesol, retinoids like 13-cis retinoic acid (isotretinoin), all-trans-retinoic acid, adapalene, their salts or derivatives, androgen inhibitors like spironolactone and cyproterone, antibiotics, preferably clindamycin, erythromycin and tetracycline, zinc or zinc salts, and antiandrogens, 5-alpha-reductase inhibitors, D-panthenol, alpha-hydroxy acids, such as e.g. salicylic acid and lactic acid, pyruvic (alfa-keto acid) acids, aliphatic dicarboxylic acids, such as e.g. azelaic acid, L-carnitine, bakuchiol, 1,2-decanediol, senkyunolide-A and senkyunolide-A containing Apium graveolens seed oil, Tetraselmis extract, Quillaja saponaria extract, Enantia chlorantha bark extract, Spiraea ulmaria extract, butyl avocate, vitamin B6 (also known as pyridoxine) or its salts or derivatives, vitamin B3 (also known as niacin or nicotinic acid) or its salts or derivatives, benzoylperoxide, phloretin, Camellia sinensis extract and contained polyphenols such as e.g. epigallocatechin-3-gallate, red clover (Trifolium pretense) extract, soybean (Glycine soja) seed extract, isoflavonoids or isoflavonoid containing extracts, preferably biochanin A, genistein, daidzein, genistin, and daizin.

Matrix-Metalloproteinase inhibitors (MMPI): A preferred homecare product composition or cosmetic or pharmaceutical composition according to the present invention comprises one or more matrix-metalloproteinase inhibitors, especially those inhibiting matrix-metalloproteinases enzymatically cleaving collagen, selected from the group consisting of: ursolic acid, retinyl palmitate, propyl gallate, precocenes, 6-hydroxy-7-methoxy-2,2-dimethyl-1(2H)-benzopyran, 3,4-dihydro-6-hydroxy-7-methoxy-2,2-dimethyl-1(2H)-benzopyran, benzamidine hydrochloride, the cysteine proteinase inhibitors N-ethylmalemide and epsilon-amino-n-caproic acid of the serinprotease inhibitors: phenylmethylsufonylfluoride, collhibin (company Pentapharm; INCI: hydrolysed rice protein), oenotherol (company Soliance; INCI: propylene glycol, aqua, Oenothera biennis root extract, ellagic acid and ellagitannins, for example from pomegranate), phosphoramidone hinokitiol, EDTA, galardin, EquiStat (company Collaborative Group; apple fruit extract, soya seed extract, ursolic acid, soya isoflavones and soya proteins), sage extracts, MDI (company Atrium; INCI: glycosaminoglycans), fermiskin (company Silab/Mawi; INCI: water and lentinus edodes extract), actimp 1.9.3 (company Expanscience/Rahn; INCI: hydrolysed lupine protein), lipobelle soyaglycone (company Mibelle; INCI: alcohol, polysorbate 80, lecithin and soy isoflavones), extracts from green and black tea and further plant extracts, proteins or glycoproteins from soya, hydrolysed proteins from rice, pea or lupine, plant extracts which inhibit MMPs, preferably extracts from shitake mushrooms, extracts from the leaves of the Rosaceae family, sub-family Rosoideae, quite particularly extracts of blackberry leaf, as e.g. SymMatrix (company Symrise, INCI: Maltodextrin, Rubus Fruticosus (Blackberry) Leaf Extract). Preferred actives of are selected from the group consisting of retinyl palmitate, ursolic acid, extracts from the leaves of the Rosaceae family, sub-family Rosoideae, genistein and daidzein.

Skin-moisturizing agents: A preferred cosmetic or pharmaceutical composition according to the present invention comprises one or more skin-moisturizing agents. Preferred skin moisturizing agents are selected from the group consisting of alkane diols or alkane triols comprising 3 to 12 carbon atoms, preferably C₃-C₁₀-alkane diols and C₃-C₁₀-alkane triols. More preferably the skin moisturizing agents are selected from the group consisting of: glycerol, 1,2-propylene glycol, 1,2-butylene glycol, 1,3-butylene glycol, 1,2-pentanediol, 1,2-hexanediol, 1,2-heptanediol, 1,2-octanediol and 1,2-decanediol.

Glycosaminoglycan stimulators: A preferred homecare product composition or cosmetic or pharmaceutical composition according to the present invention comprises one or more substances stimulating the synthesis of glycosaminoglycans which are selected from the group consisting of hyaluronic acid and derivatives or salts, Subliskin (Sederma, INCI: Sinorhizobium meliloti Ferment Filtrate, Cetyl Hydroxyethylcellulose, Lecithin), Hyalufix (BASF, INCI: Water, Butylene Glycol, Alpinia galanga leaf extract, Xanthan Gum, Caprylic/Capric Triglyceride), Stimulhyal (Soliance, INCI: Calcium ketogluconate), Syn-Glycan (DSM, INCI: Tetradecyl Aminobutyroylvalylaminobutyric Urea Trifluoroacetate, Glycerin, Magnesium chloride), Kalpariane (Biotech Marine), DC Upregulex (Distinctive Cosmetic Ingredients, INCI: Water, Butylene Glycol, Phospholipids, Hydrolyzed Sericin), glucosamine, N-acetyl glucosamine, retinoids, preferably retinol and vitamin A, Arctium lappa fruit extract, Eriobotrya japonica extract, Genkwanin, N-Methyl-L-serine, (−)-alpha-bisabolol or synthetic alpha-bisabolol such as e.g. Dragosantol and Dragosantol 100 from Symrise, oat glucan, Echinacea purpurea extract and soy protein hydrolysate. Preferred actives are selected from the group consisting of hyaluronic acid and derivatives or salts, retinol and derivatives, (−)-alpha-bisabolol or synthetic alpha-bisabolol such as e.g. Dragosantol and Dragosantol 100 from Symrise, oat glucan, Echinacea purpurea extract, Sinorhizobium Meliloti Ferment Filtrate, Calcium ketogluconate, Alpinia galanga leaf extract and tetradecyl aminobutyroylvalylaminobutyric urea trifluoroacetate.

TRPV1 antagonists: A preferred cosmetic or pharmaceutical composition according to the present invention comprises one or more TRPV1 antagonsits. Suitable compounds which reduce the hypersensitivity of skin nerves based on their action as TRPV1 antagonists, encompass e.g. trans-4-tert-butyl cyclohexanol, or indirect modulators of TRPV1 by an activation of the p-receptor, e.g. acetyl tetrapeptide-15, are preferred.

Plant extracts: A preferred homecare product composition or cosmetic or pharmaceutical composition according to the present invention comprises one or more plant extracts. Plant extracts, special highly active plant extract fractions and also highly pure active substances isolated from plant extracts can also be used in the homecare product composition or the cosmetic or pharmaceutical composition according to the present invention. Extracts, fractions and active substances from camomile, aloe vera, Commiphora species, Rubia species, willows, willow-herb, ginger, marigold, arnica, Glycyrrhiza species, Echinacea species, Rubus species and pure substances such as inter alia bisabolol, apigenin, apigenin-7-glucoside, gingerols such as [6]-gingerol, paradols such as [6]-paradol, boswellic acid, phytosterols, glycyrrhizine, glabridin or licochalcone A are particularly preferred.

Anti-inflammatory and/or redness and/or itch ameliorating agents: The cosmetic or pharmaceutical composition according to the present invention preferably also contains anti-inflammatory and/or redness and/or itch ameliorating ingredients, in particular steroidal substances of the corticosteroid type selected from the group consisting of hydrocortisone, dexamethasone, dexamethasone phosphate, methyl prednisolone or cortisone, are advantageously used as anti-inflammatory active ingredients or active ingredients to relieve reddening and itching, the list of which can be extended by the addition of other steroidal anti-inflammatories. Non-steroidal anti-inflammatories can also be used. More particularly:
(i) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone,
(ii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone, anthranilic acid derivatives, in particular avenanthramides,
(iii) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea, or single active compounds thereof as for example natural alpha-bisabolol, synthetic bisabolol, apigenin, apigenin-7-glucoside, gingerols, shogaols, gingerdiols, dehydrogingerdiones, paradols, especially natural or synthetic 6-paradol, naturally occuring avenanthramides, preferably avenanthramide A, avenanthramide B, avenanthramide C, avenanthramide D, avenanthramide E, non-natural or non-naturally occuring avenanthramides, preferably dihydroavenanthramide D, dihydroavenanthramide E, tranilast, boswellic acid, phytosterols, glycyrrhizin, glabridin, sclareolide and licochalcone A; preferably selected from the group consisting of natural alpha-bisabolol, synthetic bisabolol, natural avenanthramides, non-natural avenanthramides, preferably dihydroavenanthramide D, ginger extract, gingerols, shogaols, gingerdiols, dehydrogingerdiones, paradols, especially natural or synthetic 6-paradol, boswellic acid, phytosterols, glycyrrhizin, and licochalcone A, and/or allantoin, sclareolide, panthenol, (pseudo-)ceramides [preferably Ceramide 2, hydroxypropyl bispalmitamide M EA, cetyloxypropyl glyceryl methoxypropyl myristamide, N-(l-hexadecanoyl)-4-hydroxy-L-proline (1-hexadecyl) ester, hydroxyethyl palmityl oxyhydroxypropyl palmitamide], phytosterols, chitosan, and β-glucans, in particular 1,3-1,4-glucan from oats,
(iv) histamine receptor antagonists, serine protease inhibitors (e.g. of Soy extracts), TRPV1 antagonists (e.g. 4-t-Butylcyclohexanol), NK1 antagonists (e.g. Aprepitant, Hydroxyphenyl Propamidobenzoic Acid), cannabinoid receptor agonists (e.g. Palmitoyl Ethanolamine) and TRPV3 antagonists.

Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Anthranilic acid derivatives are preferred anti-itch ingredients in a composition according to the present invention.

Also useful are natural or naturally occurring anti-inflammatory mixtures of substances or mixtures of substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea; preferably selected from the group consisting of extracts or fractions from camomile, Aloe vera, oats, calendula, arnica, honeysuckle, rosemary, witch hazel, ginger or Echinacea, and/or pure substances, preferably alpha-bisabolol, apigenin, apigenin-7-glucoside, gingerols, shogaols, gingerdiols, dehydrogingerdiones, paradols, natural or naturally occuring avenanthramides, preferably tranilast, avenanthramide A, avenanthramide B, avenanthramide C, non-natural or non-naturally occuring avenanthramides, preferably dihydroavenanthramide D, dihydroavenanthramide E, avenanthramide D, avenan-thramide E, avenanthramide F, boswellic acid, phytosterols, glycyrrhizin, glabridin and licochalcone A; preferably selected from the group consisting of alpha-bisabolol, natural avenanthramides, non-natural avenanthramides, preferably dihydroavenanthramide D (as described in WO 2004 047833 A1), boswellic acid, phytosterols, glycyrrhizin, and licochalcone A, and/or allantoin, panthenol, lanolin, (pseudo-)ceramides [preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide], glycosphingolipids, phytosterols, chitosan, mannose, lactose and ß-glucans, in particular 1,3->1,4-ß-glucan from oats.

When bisabolol is used in the context of the present invention it can be of natural or synthetic origin, and is preferably “alpha-bisabolol”. Preferably, the bisabolol used is synthetically prepared or natural (−)-alpha-bisabolol and/or synthetic mixed-isomer alpha-bisabolol. If natural (−)-alpha-bisabolol is used, this can also be employed as a constituent of an essential oil or of a plant extract or of a fraction thereof, for example as a constituent of (fractions of) oil or extracts of camomile or of Vanillosmopsis (in particular Vanillosmopsis erythropappa or Vanillosmopsis arborea). Synthetic alpha-bisabolol is obtainable, for example, under the name “Dragosantol” from Symrise.

In case ginger extract is used in the context of the present invention, preferably extracts of the fresh or dried ginger root are used which are prepared by extraction with methanol, ethanol, iso-propanol, acetone, ethyl acetate, carbon dioxide (CO₂), hexane, methylene chloride, chloroform or other solvents or solvent mixtures of comparable polarity. The extracts are characterized by the presence of active skin irritation-reducing amounts of constituents such as e.g. gingerols, shogaols, gingerdiols, dehydrogingerdiones and/or paradols.

Moisturising and/or moisture-retaining substances: Itching occurs with particular intensity when the skin is dry. The use of skin-moisturising and/or moisture-retaining substances can significantly alleviate itching. The cosmetic or pharmaceutical composition according to the present invention can therefore advantageously also contain one or more of the following moisturising and/or moisture-retaining substances: sodium lactate, urea, urea derivatives, alcohols, glycerol, diols such as propylene glycol, hexylene glycol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulphate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

Lenitive substances: The cosmetic or pharmaceutical composition according to the present invention can also contain advantageously one or more lenitive substances, wherein any lenitive substances can be used which are suitable or customary in cosmetic or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, Arnica monatana, Glycyrrhiza species, algae, seaweed and Calendula officinalis, and vegetable oils such as sweet almond oil, baobab oil, olive oil and panthenol, Laureth-9, Trideceth-9 and 4-t-butylcyclohexanol.

Desquamating agents: The homecare product composition or the cosmetic or pharmaceutical composition according to the present invention preferably contains one or more desquamating agents. The expression “desquamating agent” is understood to mean any compound capable of acting:
either directly on desquamation by promoting exfoliation, such as β-hydroxy acids, in particular salicylic acid and its derivatives (including 5-n-octanoylsalicylic acid); α-hydroxy acids, such as glycolic, citric, lactic, tartaric, malic or mandelic acids; urea; gentisic acid; oligofucoses; cinnamic acid; extract of Sophora japonica; resveratrol and some derivatives of jasmonic acid;
or on the enzymes involved in the desquamation or the degradation of the corneodesmosomes, glycosidases, stratum corneum chymotryptic enzyme (SCCE) or other proteases (trypsin, chymotrypsin-like). There may be mentioned agents chelating inorganic salts: EDTA; N-acyl-N,N′,N′-ethylenediaminetriacetic acid; aminosulphonic compounds and in particular (N-2-hydroxyethylpiperazine-N-2-ethane)sulphonic acid (HEPES); derivatives of 2-oxothiazolidine-4-carboxylic acid (procysteine); derivatives of alpha-amino acids of the glycine type (as described in EP-0 852 949, and sodium methylglycine diacetate marketed by BASF under the trade name TRILON M); honey; sugar derivatives such as 0-octanoyl-6-D-maltose and N-acetylglucosamine; chestnut extracts such as those marketed by the company SILAB under the name Recoverine®, prickly pear extracts such as those marketed under the name Exfolactive® by the company SILAB, or Phytosphingosine SLC® (phytosphingosine grafted with a salicylic acid) marketed by the company Degussa.

Desquamating agents suitable for the invention may be chosen in particular from the group comprising sulphonic acids, calcium chelators, α-hydroxy acids such as glycolic, citric, lactic, tartaric, malic or mandelic acids; ascorbic acid and its derivatives such as ascorbyl glucoside and magnesium ascorbyl phosphate; nicotinamide; urea; (N-2-hydroxyethylpiperazine-N-2-ethane)sulphonic acid (HEPES), β-hydroxy acids such as salicylic acid and its derivatives, retinoids such as retinol and its esters, retinal, retinoic acid and its derivatives, chestnut or prickly pear extracts, in particular marketed by SILAB; reducing compounds such as cysteine or cysteine precursors.

Desquamating agents which can be used are also nicotinic acid and its esters and nicotinamide, also called vitamin B3 or vitamin PP, and ascorbic acid and its precursors.

Antidandruff substances: In addition, the cosmetic or pharmaceutical composition according to the present invention can also advantageously be used in combination with one or more anti-dandruff substances, including triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate, Propanediol Monocaprylate or combinations of said substances, which are used inter alia against dandruff.

Further suitable anti-dandruff agents are Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

(Metal) chelating agents: A combination with one or more (metal) chelating agents can also be advantageous used in the homecare product composition or cosmetic or pharmaceutical composition according to the present invention, wherein any metal chelating agents can be used which are suitable or customary in homecare product composition or cosmetic or pharmaceutical applications. Preferred (metal) chelating agents include α-hydroxy fatty acids, phytic acid, lactoferrin, α-hydroxy acids, such as inter alia gluconic acid, glyceric acid, glycolic acid, isocitric acid, citric acid, lactic acid, malic acid, mandelic acid, tartaric acid, as well as humic acids, bile acids, bile extracts, bilirubin, biliverdin or EDTA, EGTA and their derivatives. The use of one or more chelating agent(s) improves the stability of the composition according to the present invention.

Emulsifiers: In addition, the homecare product composition or cosmetic or pharmaceutical composition according to the present invention can also advantageously contain one or more emulsifiers, including for example:
products of the addition of 2 to 30 mol ethylene oxide and/or 0 to 5 mol propylene oxide onto linear C₈₋₂₂ fatty alcohols, onto C₁₂₋₂₂ fatty acids and onto alkyl phenols containing 8 to 15 carbon atoms in the alkyl group;
C_{12/18} fatty acid monoesters and diesters of addition products of 1 to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
addition products of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 to 15 mol ethylene oxide onto castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);
mono-, di and trialkyl phosphates and mono-, di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol and/or mixed esters of C₆₋₂₂ fatty acids, methyl glucose and polyols, preferably glycerol or polyglycerol;
polyalkylene glycols; and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products. They are homologue mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations. The preferred emulsifiers are described in more detail as follows:

Partial glycerides: Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan esters: Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol esters: Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Poly-glyceryl-3 Methylglucose Distearate (Tego Care® 450), Polyglyceryl-3 Beeswax (Cera Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Anionic emulsifiers: Typical anionic emulsifiers are aliphatic C12 to C22 fatty acids, such as palmitic acid, stearic acid or behenic acid for example, and C12 to C22 dicarboxylic acids, such as azelaic acid or sebacic acid for example.

Amphoteric emulsifiers: Other suitable emulsifiers are amphboteric or zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine.

Surfactants: For the preparation of solid surfactant composition, the homecare product composition or cosmetic or pharmaceutical composition according to the present invention preferably includes one or more surfactant(s) which is/are commonly used therefor. Suitable surfactants include Sodium Laureth sulfate, Cocamidopropyl Betaine, Sodium Stearate, Sodium Lauryl Sulfate, Glycol distearate, Coco-Glucoside, Sodium Coco-Sulfate, Sodium Cocoate, Coco-Betaine, Cocamide MEA, Lauryl Glucoside, PEG-7 Glyceryl Cocoate, Decyl Glucoside, Potassium Lauryl Sulfate, Sodium Palmate, Sodium Palm Kernelate, Sodium Cocoyl Glutamate, Disodium Laureth Sulfosuccinate, Sodium lauroyl sarcosinate, Sodium Lauroyl Sarcosinate, Sodium Cocoyl Isethionate, Sodium cocoamphoacetate, Sodium Methyl Cocoyl Taurate, Caprylyl/Capryl Glucoside, Disodium Lauryl Sulfosuccinate, Sodium Lauryl Sulfosuccinate, PPG-5-ceteth-20, Polyglyceryl-4 Laurate, Sodium Lauryl Sulfoacetate, Sodium Lauryl Sulfoacetate, Sodium Myristoyl Glutamate, Alpha Olefin Sulfonate, Diethylhexyl Sodium Sulfosuccinate, Ammonium Cocoyl Isethionate, Sodium Oleoyl Sarcosinate, Ammonium Cocoyl Sarcosinate, or mixtures of one or more of the afore-said surfactants.

Preferred surfactants for the preparation of solid surfactant compositions according to the present invention are selected from the group consisting of Sodium Stearate, Sodium Lauryl Sulfate, Coco-Glucoside, Sodium Coco-Sulfate, Sodium Cocoate, Coco-Betaine, Lauryl Glucoside, Decyl Glucoside, Potassium Lauryl Sulfate, Sodium Palmate, Sodium Palm Kernelate, Sodium Cocoyl Glutamate, Disodium Laureth sulfosuccinate, Sodium lauroyl sarcosinate, Sodium Lauroyl Sarcosinate, Sodium Cocoyl Isethionate, Sodium Cocoamphoacetate, Sodium Methyl Cocoy Taurate, Caprylyl/Capryl glucoside, Disodium Lauryl Sulfosuccinate, and mixtures of two or more of the afore-said surfactants.

Preservatives: For preservative purposes, the homecare product composition or cosmetic or pharmaceutical composition according to the present invention preferably includes one or more preservatives which are suitable or customary in homecare product composition or cosmetic or pharmaceutical composition. Suitable and advantageously preservatives are, for example, phenoxyethanol, formaldehyde solution, parabens, pentanediol or sorbic acid.

Green and synthetic polymers: The homecare product composition or cosmetic or pharmaceutical composition according to the present invention preferably includes one or more preservatives. Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lame-quat® L, GrUnau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copoly-mers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar® CBS, Jaguar® C-17, Jaguar® C-16 of Celanese, quaternized ammonium salt polymers such as, for example, Mirapol® A-15, Mirapol® AD-1, Mirapol® AZ-1 of Miranol and the various polyquaternium types (for example 6, 7, 32 or 37) which can be found in the market under the tradenames Rheocare® CC or Ultragel® 300. Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Thickening agents and/or rheology additives: The homecare product composition or cosmetic or pharmaceutical composition according to the present invention preferably includes one or more thickening agents and/or rheology additives. Suitable thickeners are polymeric thickeners, such as Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates and electrolytes, such as sodium chloride and ammonium chloride.

Perfume oils and/or fragrances: The homecare product composition or cosmetic or pharmaceutical composition according to the present invention preferably includes one or more perfume oils and/or fragrances. Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, beta-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Anti-cellulite agent: The cosmetic or pharmaceutical composition according to the present invention preferably includes one or more anti-cellulite agents. Anti-cellulite agents and lipolytic agents are preferably selected from the group consisting of beta-adrenergic receptor agonists such as synephrine and its derivatives, and cyclohexyl carbamates. Agents enhancing or boosting the activity of anti-cellulite agents, in particular agents which stimulate and/or depolarise C nerve fibres, are preferably selected from the group consisting of capsaicin and derivatives thereof, vanillyl-nonylamid and derivatives thereof, L-carnitine, coenzym A, isoflavonoides, soy extracts, ananas extract and conjugated linoleic acid.

Fat enhancing agents: The cosmetic or pharmaceutical composition according to the present invention preferably includes one or more fat enhancing agents and/or adipogenic agents as well as agents enhancing or boosting the activity of fat enhancing agents. A fat enhancing agent is for example hydroxymethoxyphenyl propylmethylmethoxybenzofuran (trade name: Sym3D®).

Oil bodies: The homecare product composition or cosmetic or pharmaceutical composition according to the present invention preferably includes one or more oil bodies. Suitable oil bodies, which form constituents of the O/W emulsions, are, for example, Guerbet alcohols based on fatty alcohols having 6 to 18, preferably 8 to 10, carbon atoms, esters of linear C5-C22 fatty acids with linear or branched C5-C22 fatty alcohols or esters of branched C1-C13 carboxylic acids with linear or branched C6-C22 fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C6-C22 fatty acids with branched alcohols, in particular 2-ethylhexanol, esters of C18-C38 alkylhydroxy carboxylic acids with linear or branched C6-C22 fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C6-C10 fatty acids, liquid mono-/di-/triglyceride mixtures based on C6-C18 fatty acids, esters of C6-C22 fatty alcohols and/or Guerbet alcohols with aromatic carboxylic acids, in particular benzoic acid, esters of C2-C12 dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched CC-C22 fatty alcohol carbonates, such as, for example, Dicaprylyl Carbonate (Cetiol® CC), Guerbet carbonates, based on fatty alcohols having 6 to 18, preferably 8 to 10, carbon atoms, esters of benzoic acid with linear and/or branched C6-C22-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, squalane, squalene or dialkylcyclohexanes.

Superfatting agents and/or consistency factors: The homecare product composition or cosmetic or pharmaceutical composition according to the present invention preferably includes one or more superfatting agents and/or consistency factors. Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers. The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Pearlizing waxes: The homecare product composition or cosmetic or pharmaceutical composition according to the present invention preferably includes one or more pearlizing waxes. Suitable pearlising waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Silicones: In order to impart a silky, spreadable, and luxurious texture and to make skin look and feel smoother, and additionally to improve processability (antifoaming) the homecare product composition or cosmetic or pharmaceutical composition according to the present invention preferably includes one or more silicones or silicone deratives. Suitable silicones can be chosen from the group consisting of: Acefylline Methylsilanol Mannuronate, Acetylmethionyl Methylsilanol Elastinate Acrylates/Behenyl, Acrylate/Dimethicone Methacrylate Copolymer, Acrylates/Behenyl Methacrylate/Dimethicone Methacrylate Copolymer, Acrylates/Bis-Hydroxypropyl Dimethicone Crosspolymer, Acrylates/Dimethicone Copolymer, Acrylates/Dimethicone Methacrylate/Ethylhexyl Acrylate Copolymer, Acrylates/Dimethiconol Acrylate Copolymer, Acrylates/Ethylhexyl Acrylate/Dimethicone Methacrylate Copolymer, Acrylates/Octylacrylamide/Diphenyl Amodimethicone Copolymer, Acrylates/Polytrimethylsiloxymethacrylate Copolymer, Acrylates/Propyl Trimethicone Methacrylate Copolymer, Acrylates/Stearyl Acrylate/Dimethicone Methacrylate Copolymer, Acrylates/Tridecyl Acrylate/Triethoxysilylpropyl Methacrylate/Dimethicone Methacrylate Copolymer, Acrylates/Trifluoropropylmethacrylate/Polytrimethyl Siloxymethacrylate Copolymer, Amino Bispropyl Dimethicone, Aminoethylaminopropyl Dimethicone, Aminopropyl Dimethicone, Aminopropyl Phenyl Trimethicone, Aminopropyl Triethoxysilane, Ammonium Dimethicone PEG-7 Sulfate, Amodimethicone, Amodimethicone Hydroxystearate, Amodimethicone/Silsesquioxane Copolymer, Ascorbyl Carboxydecyl Trisiloxane, Ascorbyl Methylsilanol Pectinate, Behenoxy Dimethicone, Behentrimonium Dimethicone PEG-8 Phthalate, Behenyl Dimethicone, Bisamino PEG/PPG-41/3 Aminoethyl PG-Propyl Dimethicone, Bis-Aminopropyl/Ethoxy Aminopropyl Dimethicone, Bis(Butylbenzoate) Diaminotriazine Aminopropyltrisiloxane, Bis-Butyldimethicone Polyglyceryl-3, Bis-Butyloxyamodimethicone/PEG-60 Copolymer, Bis(C13-15 Alkoxy) Hydroxybutamidoamodimethicone, Bis(C13-15 Alkoxy) PG-Amodimethicone, Bis-(C1-8 Alkyl Lauroyl Lysine Decylcarboxamide) Dimethicone, Bis-Cetyl Cetyl Dimethicone, Bis-Cetyl/PEG-8 Cetyl PEG-8 Dimethicone, Bis-Diphenylethyl Disiloxane, Bis-Ethyl Ethyl Methicone, Bis-Gluconamidoethylaminopropyl Dimethicone, Bis-Hydrogen Dimethicone, Bis-Hydroxyethoxypropyl Dimethicone Bis-Hydroxylauryl, Dimethicone/IPDI Copolymer, Bis-Hydroxy/Methoxy Amodimethicone, Bis-Hydroxypropyl Dimethicone Behenate, Bis-Hydroxypropyl Dimethicone/SMDI Copolymer, Bis-Isobutyl PEG-14/Amodimethicone Copolymer, Bis-Isobutyl PEG-15/Amodimethicone Copolymer, Bis-Isobutyl PEG/PPG-20/35/Amodimethicone Copolymer, Bis-Isobutyl PEG/PPG-10/7/Dimethicone Copolymer, Bis-Isobutyl PEG-24/PPG-7/Dimethicone Copolymer, Bis-PEG-1 Dimethicone, Bis-PEG-4 Dimethicone, Bis-PEG-8 Dimethicone, Bis-PEG-12 Dimethicone, Bis-PEG-20 Dimethicone, Bis-PEG-12 Dimethicone Beeswax, Bis-PEG-12 Dimethicone Candelillate, Bis-PEG-15 Dimethicone/IPDI Copolymer, Bis-PEG-15 Methyl Ether Dimethicone, Bis-PEG-18 Methyl Ether Dimethyl Silane, Bis-PEG/PPG-14/14 Dimethicone, Bis-PEG/PPG-15/5 Dimethicone, Bis-PEG/PPG-18/6 Dimethicone, Bis-PEG/PPG-20/20 Dimethicone, Bis-PEG/PPG-16/16 PEG/PPG-16/16 Dimethicone, Bis-PEG/PPG-20/5 PEG/PPG-20/5 Dimethicone, Bisphenylhexamethicone, Bis-Phenylpropyl Dimethicone, Bispolyethylene Dimethicone, Bis-(Polyglyceryl-3 Oxyphenylpropyl) Dimethicone, Bis-(Polyglyceryl-7 Oxyphenylpropyl) Dimethicone, Bis-PPG-15 Dimethicone/IPDI Copolymer, Bis(PPG-7 Undeceneth-21) Dimethicone, Bis-Stearyl Dimethicone, Bis-Trimethoxysilylethyl Tetramethyldisiloxyethyl Dimethicone, Bis-Vinyldimethicone, Bis-Vinyl Dimethicone/Dimethicone Copolymer, Borage Seed Oil PEG-7 Dimethicone Esters, Butyl Acrylate/C6-14 Perfluoroalkylethyl Acrylate/Mercaptopropyl Dimethicone Copolymer, Butyl Acrylate/Hydroxypropyl Dimethicone Acrylate Copolymer, Butyl Dimethicone Acrylate/Cyclohexylmethacrylate/Ethylhexyl Acrylate Copolymer, Butyldimethicone Methacrylate/Methyl Methacrylate Crosspolymer, t-Butyl Dimethyl Silyl Grape Seed Extract, Butyl Polydimethylsiloxyl Ethylene/Propylene/Vinylnorbornene Copolymer, C6-8 Alkyl C3-6 Alkyl Glucoside Dimethicone, C20-24 Alkyl Dimethicone, C24-28 Alkyl Dimethicone, C26-28 Alkyl Dimethicone, C30-45 Alkyl Dimethicone, C30-60 Alkyl Dimethicone, C32 Alkyl Dimethicone, C30-45 Alkyl Dimethicone/Polycyclohexene Oxide Crosspolymer, C26-28 Alkyldimethylsilyl Polypropylsilsesquioxane, C30-45 Alkyldimethylsilyl Polypropylsilsesquioxane, C20-24 Alkyl Methicone, C24-28 Alkyl Methicone, C26-28 Alkyl Methicone, C30-45 Alkyl Methicone, C20-28 Alkyl Perfluorodecylethoxy Dimethicone, C26-54 Alkyl Tetradecyl Dimethicone, Capryl Dimethicone, Caprylyl Dimethicone Ethoxy Glucoside, Caprylyl Methicone, Caprylyl Trimethicone, Carboxydecyl Trisiloxane, Castor Oil Bis-Hydroxypropyl Dimethicone Esters Cerotyl Dimethicone, Cetearyl Dimethicone Crosspolymer, Cetearyl Dimethicone/Vinyl Dimethicone Crosspolymer, Cetearyl Methicone, Cetrimonium Carboxydecyl PEG-8 Dimethicone, Cetrimonium Dimethicone PEG-7 Phthalate, Cetyl Behenyl Dimethicone, Cetyl Dimethicone, Cetyl Dimethicone/Bis-Vinyldimethicone Crosspolymer, Cetyl Hexacosyl Dimethicone, Cetyloxy Dimethicone, Cetyl PEG-8 Dimethicone, Cetyl PEG/PPG-15/15 Butyl Ether Dimethicone, Cetyl PEG/PPG-7/3 Dimethicone, Cetyl PEG/PPG-10/1 Dimethicone, Cetyl Triethylmonium Dimethicone PEG-8 Phthalate, Cetyl Triethylmonium Dimethicone PEG-8 Succinate, Copper Acetyl Tyrosinate Methylsilanol, Copper PCA Methylsilanol, C4-14 Perfluoroalkylethoxy Dimethicone, Cycloethoxymethicone, Cycloheptasiloxane, Cyclohexasiloxane, Cyclomethicone, Cyclopentasiloxane, Cyclophenylmethicone, Cyclotetrasiloxane, Cyclovinylmethicone, Cystine Bis-PG-Propyl Silanetriol, DEA PG-Propyl PEG/PPG-18/21 Dimethicone, Diisostearoyl Trimethylolpropane Siloxy Silicate, Dilauroyl Trimethylolpropane Siloxy Silicate, Dilinoleamidopropyl Dimethylamine Dimethicone PEG-7 Phosphate, Dimethicone, Dimethicone Crosspolymer, Dimethicone Crosspolymer-3, Dimethicone/Divinyldimethicone/Silsesquioxane Crosspolymer, Dimethicone Ethoxy Glucoside, Dimethicone Hydroxypropyl Trimonium Chloride, Dimethicone/Mercaptopropyl Methicone Copolymer, Dimethicone PEG-15 Acetate Dimethicone PEG-8 Adipate, Dimethicone PEG-7 Avocadoate, Dimethicone PEG-8 Avocadoate, Dimethicone PEG-8 Beeswax, Dimethicone PEG-8 Benzoate, Dimethicone PEG-8 Borageate, Dimethicone PEG-7 Cocoate, Dimethicone/PEG-10 Crosspolymer, Dimethicone/PEG-10/15 Crosspolymer, Dimethicone/PEG-15 Crosspolymer, Dimethicone PEG-7 Isostearate, Dimethicone PEG-8 Isostearate, Dimethicone PEG-7 Lactate, Dimethicone PEG-8 Lanolate, Dimethicone PEG-8 Laurate, Dimethicone PEG-8 Meadowfoamate, Dimethicone PEG-7 Octyldodecyl Citrate, Dimethicone PEG-7 Olivate, Dimethicone PEG-8 Olivate, Dimethicone PEG-7 Phosphate, Dimethicone PEG-8 Phosphate, Dimethicone PEG-10 Phosphate, Dimethicone PEG-7 Phthalate, Dimethicone PEG-8 Phthalate, Dimethicone PEG-8 Polyacrylate, Dimethicone PEG/PPG-20/23 Benzoate, Dimethicone PEG/PPG-7/4 Phosphate, Dimethicone PEG/PPG-12/4 Phosphate, Dimethicone PEG-7 Succinate, Dimethicone PEG-8 Succinate, Dimethicone PEG-7 Sulfate, Dimethicone PEG-7 Undecylenate, Dimethicone PG-Diethylmonium Chloride, Dimethicone/Phenyl Vinyl Dimethicone Crosspolymer, Dimethicone/Polyglycerin-3 Crosspolymer, Dimethicone/PPG-20 Crosspolymer, Dimethicone Propylethylenediamine Behenate, Dimethicone Propyl PG-Betaine, Dimethicone/Silsesquioxane Copolymer, Dimethicone Silylate, Dimethicone/Vinyldimethicone Crosspolymer, Dimethicone/Vinyltrimethylsiloxysilicate Crosspolymer, Dimethiconol, Dimethiconol Arginine, Dimethiconol Beeswax, Dimethiconol Behenate, Dimethiconol Borageate, Dimethiconol Candelillate, Dimethiconol Carnaubate, Dimethiconol Cysteine, Dimethiconol Dhupa Butterate, Dimethiconol Fluoroalcohol Dilinoleic Acid, Dimethiconol Hydroxystearate, Dimethiconol Illipe Butterate, Dimethiconol/IPDI Copolymer, Dimethiconol Isostearate, Dimethiconol Kokum Butterate, Dimethiconol Lactate, Dimethiconol Meadowfoamate, Dimethiconol Methionine, Dimethiconol/Methylsilanol/Silicate Crosspolymer, Dimethiconol Mohwa Butterate, Dimethiconol Panthenol, Dimethiconol Sal Butterate, Dimethiconol/Silica Crosspolymer, Dimethiconol/Silsesquioxane Copolymer, Dimethiconol Stearate, Dimethiconol/Stearyl, Methicone/Phenyl Trimethicone Copolymer, Dimethoxysilyl Ethylenediaminopropyl Dimethicone, Dimethylaminopropylamido PCA Dimethicone, Dimethyl Oxobenzo Dioxasilane, Dimethylsilanol Hyaluronate, Dioleyl Tocopheryl Methylsilanol, Diphenyl Amodimethicone, Diphenyl Dimethicone, Diphenyl Dimethicone Crosspolymer Diphenyl Dimethicone?/inyl Diphenyl Dimethicone/Silsesquioxane Crosspolymer, Diphenylethyl Benzyloxy Dilsiloxane, Diphenylisopropyl Dimethicone, Diphenylsiloxy Phenyl/Propyl Trimethicone, Diphenylsiloxy Phenyl Trimethicone Disiloxane, Disodium Amodimethicone Disuccinamide, Disodium PEG-12 Dimethicone Sulfosuccinate, Disodium PEG-8 Lauryl Dimethicone Sulfosuccinate, Divinyldimethicone/Dimethicone Copolymer, Divinyldimethicone/Dimethicone Crosspolymer, Drometrizole Trisiloxane, Ethylhexyl Acrylate/VP/Dimethicone Methacrylate Copolymer, Ethyl Methicone, Ethyl Trisiloxane, Fluoro C2-8 Alkyldimethicone, Gluconamidopropyl Aminopropyl Dimethicone, 4-(2-Beta-Glucopyranosiloxy) Propoxy-2-Hydroxybenzophenone, Glyceryl Undecyl Dimethicone, Glycidoxy Dimethicone, Hexadecyl Methicone, Hexyl Dimethicone, Hexyl Methicone, Hexyltrimethoxysilane, Hydrogen Dimethicone, Hydrogen Dimethicone/Octyl Silsesquioxane Copolymer, Hydrolyzed Collagen PG-Propyl Dimethiconol, Hydrolyzed Collagen PG-Propyl Methylsilanediol, Hydrolyzed Collagen PG-Propyl Silanetriol, Hydrolyzed Keratin PG-Propyl Methylsilanediol, Hydrolyzed Sesame Protein PG-Propyl Methylsilanediol, Hydrolyzed Silk PG-Propyl Methylsilanediol, Hydrolyzed Silk PG-Propyl Methylsilanediol Crosspolymer, Hydrolyzed Soy Protein/Dimethicone PEG-7 Acetate, Hydrolyzed Soy Protein PG-Propyl Methylsilanediol, Hydrolyzed Vegetable Protein PG-Propyl Silanetriol, Hydrolyzed Wheat Protein/Cystine Bis-PG-Propyl Silanetriol Copolymer, Hydrolyzed Wheat Protein/Dimethicone PEG-7 Acetate, Hydrolyzed Wheat Protein/Dimethicone PEG-7 Phosphate Copolymer, Hydrolyzed Wheat Protein PG-Propyl Methylsilanediol, Hydrolyzed Wheat Protein PG-Propyl Silanetriol, Hydroxyethyl Acetomonium PG-Dimethicone, Hydroxypropyldimethicone, Hydroxypropyl Dimethicone Behenate, Hydroxypropyl Dimethicone Isostearate, Hydroxypropyl Dimethicone Stearate, Isobutylmethacrylate/Bis-Hydroxypropyl Dimethicone Acrylate Copolymer, Isobutylmethacrylate/Trifluoroethylmethacrylate/Bis-Hydroxypropyl Dimethicone Acrylate Copolymer, Isopentyl Trimethoxycinnamate Trisiloxane, Isopolyglyceryl-3 Dimethicone, Isopolyglyceryl-3 Dimethiconol, Isopropyl Titanium Triisostearate/Triethoxysilylethyl, Polydimethylsiloxyethyl Dimethicone Crosspolymer, Isostearyl Carboxydecyl PEG-8 Dimethicone, Lactoyl Methylsilanol Elastinate, Lauryl Dimethicone, Lauryl Dimethicone PEG-15 Crosspolymer, Lauryl Dimethicone PEG-10 Phosphate, Lauryl Dimethicone/Polyglycerin-3 Crosspolymer, Lauryl Methicone, Lauryl PEG-8 Dimethicone, Lauryl PEG-10 Methyl Ether Dimethicone, Lauryl PEG-9 Polydimethylsiloxyethyl Dimethicone, Lauryl PEG/PPG-18/18 Methicone, Lauryl Phenylisopropyl Methicone, Lauryl Phenylpropyl Methicone, Lauryl Polydimethylsiloxyethyl Dimethicone/Bis-Vinyldimethicone Crosspolymer, Lauryl Polyglyceryl-3 Polydimethylsiloxyethyl Dimethicone, Lauryl Trimethicone, Linoleamidopropyl PG-Dimonium Chloride Phosphate Dimethicone, Methacryloyl Propyltrimethoxysilane, Methicone, Methoxy Amodimethicone/Silsesquioxane Copolymer, Methoxycinnamidopropyl Polysilsesquioxane, Methoxycinnamoylpropyl Silsesquioxane Silicate, Methoxy PEG-13 Ethyl Polysilsesquioxane, Methoxy PEG/PPG-7/3 Aminopropyl Dimethicone, Methoxy PEG/PPG-25/4 Dimethicone, Methoxy PEG-10 Propyltrimethoxysilane, Methyleugenyl PEG-8 Dimethicone, Methylpolysiloxane Emulsion, Methylsilanol Acetylmethionate, Methylsilanol Acetyltyrosine, Methylsilanol Ascorbate, Methylsilanol Carboxymethyl Theophylline, Methylsilanol Carboxymethyl Theophylline Alginate, Methylsilanol Elastinate, Methylsilanol Glycyrrhizinate, Methylsilanol Hydroxyproline, Methylsilanol Hydroxyproline Aspartate, Methylsilanol Mannuronate, Methylsilanol PCA, Methylsilanol PEG-7 Glyceryl Cocoate, Methylsilanol/Silicate Crosspolymer, Methylsilanol Spirulinate, Methylsilanol Tri-PEG-8 Glyceryl Cocoate, Methyl Trimethicone, Methyltrimethoxysilane, Myristylamidopropyl Dimethylamine Dimethicone PEG-7 Phosphate, Myristyl Methicone, Myristyl Trisiloxane, Nylon-611/Dimethicone Copolymer, PCA Dimethicone, PEG-7 Amodimethicone, PEG-8 Amodimethicone, PEG-8 Cetyl Dimethicone, PEG-3 Dimethicone, PEG-6 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG-10 Dimethicone Crosspolymer, PEG-12 Dimethicone Crosspolymer, PEG-8 Dimethicone Dimer Dilinoleate, PEG-8 Dimethicone/Dimer Dilinoleic Acid Copolymer, PEG-10 Dimethicone/Vinyl Dimethicone Crosspolymer, PEG-8 Distearmonium Chloride PG-Dimethicone, PEG-10/Lauryl Dimethicone Crosspolymer, PEG-15/Lauryl Dimethicone Crosspolymer, PEG-15/Lauryl Polydimethylsiloxyethyl Dimethicone Crosspolymer, PEG-8 Methicone, PEG-6 Methicone Acetate, PEG-6 Methyl Ether Dimethicone, PEG-7 Methyl Ether Dimethicone, PEG-8 Methyl Ether Dimethicone, PEG-9 Methyl Ether Dimethicone, PEG-10 Methyl Ether Dimethicone, PEG-11 Methyl Ether Dimethicone, PEG-32 Methyl Ether Dimethicone, PEG-8 Methyl Ether Triethoxysilane, PEG-10 Nonafluorohexyl Dimethicone Copolymer, PEG-4 PEG-12 Dimethicone, PEG-8 PG-Coco-Glucoside Dimethicone, PEG-9 Polydimethylsiloxyethyl Dimethicone, PEG/PPG-20/22 Butyl Ether Dimethicone, PEG/PPG-22/22 Butyl Ether Dimethicone, PEG/PPG-23/23 Butyl Ether Dimethicone, PEG/PPG-24/18 Butyl Ether Dimethicone, PEG/PPG-27/9 Butyl Ether Dimethicone, PEG/PPG-3/10 Dimethicone, PEG/PPG-4/12 Dimethicone, PEG/PPG-6/4 Dimethicone, PEG/PPG-6/11 Dimethicone, PEG/PPG-8/14 Dimethicone, PEG/PPG-8/26 Dimethicone, PEG/PPG-10/2 Dimethicone, PEG/PPG-12/16 Dimethicone, PEG/PPG-12/18 Dimethicone, PEG/PPG-14/4 Dimethicone, PEG/PPG-15/5 Dimethicone, PEG/PPG-15/15 Dimethicone, PEG/PPG-16/2 Dimethicone, PEG/PPG-16/8 Dimethicone, PEG/PPG-17/18 Dimethicone, PEG/PPG-18/6 Dimethicone, PEG/PPG-18/12 Dimethicone, PEG/PPG-18/18 Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone, PEG/PPG-27/27 Dimethicone, PEG/PPG-30/10 Dimethicone, PEG/PPG-25/25 Dimethicone/Acrylates Copolymer, PEG/PPG-20/22 Methyl Ether Dimethicone, PEG/PPG-24/24 Methyl Ether Glycidoxy Dimethicone, PEG/PPG-10/3 Oleyl Ether Dimethicone, PEG/PPG-5/3 Trisiloxane, PEG-4 Trifluoropropyl Dimethicone Copolymer, PEG-8 Trifluoropropyl Dimethicone Copolymer, PEG-10 Trifluoropropyl Dimethicone Copolymer, PEG-8 Trisiloxane, Perfluorocaprylyl riethoxysilylethyl Methicone, Perfluorononyl Dimethicone, Perfluorononyl Dimethicone/Methicone/Amodimethicone Crosspolymer, Perfluorononylethyl Carboxydecyl Behenyl Dimethicone, Perfluorononylethyl Carboxydecyl Hexacosyl Dimethicone, Perfluorononylethyl Carboxydecyl Lauryl/Behenyl Dimethicone, Perfluorononylethyl Carboxydecyl Lauryl Dimethicone, Perfluorononylethyl Carboxydecyl PEG-8 Dimethicone, Perfluorononylethyl Carboxydecyl PEG-10 Dimethicone, Perfluorononylethyl Dimethicone/Methicone Copolymer, Perfluorononylethyl PEG-8 Dimethicone, Perfluorononylethyl Stearyl Dimethicone, Perfluorooctylethyl/Diphenyl Dimethicone Copolymer, Perfluorooctylethyl Triethoxysilane, Perfluorooctylethyl Trimethoxysilane, Perfluorooctylethyl Trisiloxane, Perfluorooctyl Triethoxysilane, PG-Amodimethicone, Phenethyl Dimethicone, Phenethyl Disiloxane, Phenyl Dimethicone, Phenylisopropyl Dimethicone, Phenyl Methicone, Phenyl Methiconol, Phenylpropyldimethylsiloxysilicate, Phenylpropyl Ethyl Methicone, Phenyl Propyl Trimethicone, Phenyl Propyl Trimethicone/Diphenylmethicone, Phenyl Trimethicone, Platinum Divinyldisiloxane, Polyacrylate-6, Polydiethylsiloxane, Polydimethylsiloxyethyl Dimethicone/Bis-Vinyldimethicone Crosspolymer, Polydimethylsiloxyethyl Dimethicone/Methicone Copolymer, Polydimethylsiloxy PEG/PPG-24/19 Butyl Ether Silsesquioxane, Polydimethylsiloxy PPG-13 Butyl Ether Silsesquioxane, Polyglyceryl-3 Disiloxane Dimethicone, Polyglyceryl-3/Lauryl Polydimethylsiloxyethyl Dimethicone Crosspolymer, Polyglyceryl-3 Polydimethylsiloxyethyl Dimethicone, Poly(Glycol Adipate)/Bis-Hydroxyethoxypropyl Dimethicone Copolymer, Polymethylsilsesquioxane, Polymethylsilsesquioxane/Trimethylsiloxysilicate, Polyphenylsilsesquioxane, Polypropylsilsesquioxane, Polysilicone-1, Polysilicone-2, Polysilicone-3, Polysilicone-4, Polysilicone-5, Polysilicone-6, Polysilicone-7, Polysilicone-8, Polysilicone-9, Polysilicone-10, Polysilicone-11, Polysilicone-12, Polysilicone-13, Polysilicone-14, Polysilicone-15, Polysilicone-16, Polysilicone-17, Polysilicone-18, Polysilicone-19, Polysilicone-20, Polysilicone-21, Polysilicone-18 Cetyl Phosphate, Polysilicone-1 Crosspolymer, Polysilicone-18 Stearate, Polyurethane-10, Potassium Dimethicone PEG-7 Panthenyl Phosphate, Potassium Dimethicone PEG-7 Phosphate, PPG-12 Butyl Ether Dimethicone, PPG-2 Dimethicone, PPG-12 Dimethicone, PPG-27 Dimethicone, PPG-4 Oleth-10 Dimethicone, Propoxytetramethyl Piperidinyl Dimethicone, Propyl Trimethicone, Quaternium-80, Retinoxytrimethylsilane, Silanediol Salicylate, Silanetriol, Silanetriol Arginate, Silanetriol Glutamate, Silanetriol Lysinate, Silanetriol Melaninate, Silanetriol Trehalose Ether, Silica, Silica Dimethicone Silylate, Silica Dimethyl Silylate, Silica Silylate, Silicon Carbide, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, SiliconeQuaternium-16, Silicone Quaternium-16/Glycidoxy Dimethicone Crosspolymer, Silicone Quaternium-17, Silicone Quaternium-18, Silicone Quaternium-19, Silicone Quaternium-20, Silicone Quaternium-21, Silicone Quaternium-22, Silicone Quaternium-24, Silicone Quaternium-25, Siloxanetriol Alginate, Siloxanetriol Phytate, Simethicone, Sodium Carboxydecyl PEG-8 Dimethicone, Sodium Dimethicone PEG-7 Acetyl Methyltaurate, Sodium Hyaluronate Dimethylsilanol, Sodium Lactate Methylsilanol, Sodium Mannuronate Methylsilanol, Sodium PCA Methylsilanol, Sodium PG-Propyldimethicone Thiosulfate Copolymer, Sodium PG-Propyl Thiosulfate Dimethicone, Sodium Propoxyhydroxypropyl Thiosulfate Silica, Sorbityl Silanediol, Soy Triethoxysilylpropyldimonium Chloride, Stearalkonium Dimethicone PEG-8 Phthalate, Stearamidopropyl Dimethicone, Steardimonium Hydroxypropyl Panthenyl PEG-7 Dimethicone Phosphate Chloride, Steardimonium Hydroxypropyl PEG-7 Dimethicone Phosphate Chloride, Stearoxy Dimethicone, Stearoxymethicone/Dimethicone Copolymer, Stearoxytrimethylsilane, Stearyl Aminopropyl Methicone, Stearyl Dimethicone, Stearyl/Lauryl Methacrylate Crosspolymer, Stearyl Methicone, Stearyl Triethoxysilanek, Stearyl Trimethicone, Styrene/Acrylates/Dimethicone Acrylate Crosspolymer, Styrene/Acrylates/Dimethicone Copolymer, TEA-Dimethicone PEG-7 Phosphate, Tetrabutoxypropyl Trisiloxane, Tetramethyl Hexaphenyl Tetrasiloxane, Tetramethyl Tetraphenyl Trisiloxane, Tocopheryloxypropyl Trisiloxane, Trideceth-9 PG-Amodimethicone, Triethoxycaprylylsilane, Triethoxysilylethyl Dimethicone/Methicone Copolymer, Triethoxysilylethyl Polydimethylsiloxyethyl Dimethicone, Triethoxysilylethyl Polydimethylsiloxyethyl Hexyl Dimethicone, Triethoxysilylpropylcarbamoyl Ethoxypropyl Butyl Dimethicone, Trifluoromethyl C1-4 Alkyl Dimethicone, Trifluoropropyl Cyclopentasiloxane, Trifluoropropyl Cyclotetrasiloxane, Trifluoropropyl Dimethicone, Trifluoropropyl Dimethicone/PEG-10 Crosspolymer, Trifluoropropyl Dimethicone/Trifluoropropyl Divinyldimethicone Crosspolymer, Trifluoropropyl Dimethicone/Vinyl Trifluoropropyl, Dimethicone/Silsesquioxane Crosspolymer, Trifluoropropyl Dimethiconol, Trifluoropropyldimethyl/trimethylsiloxysilicate, Trifluoropropyl Methicone, Trimethoxycaprylylsilane, Trimethoxysilyl Dimethicone, Trimethyl Pentaphenyl Trisiloxane, Trimethylsiloxyamodimethicone, Trimethylsiloxyphenyl Dimethicone, Trimethylsiloxysilicate, Trimethylsiloxysilicate/Dimethicone Crosspolymer, Trimethylsiloxysilicate/Dimethiconol Crosspolymer, Trimethylsiloxysilylcarbamoyl Pullulan, Trimethylsilyl Hydrolyzed Conchiolin Protein PG-Propyl Methylsilanediol Crosspolymer, Trimethylsilyl Hydrolyzed Silk PG-Propyl Methylsilanediol Crosspolymer, Trimethylsilyl Hydrolyzed Wheat Protein PG-Propyl Methylsilanediol Crosspolymer, Trimethylsilyl Pullulan, Trimethylsilyl Trimethylsiloxy Glycolate, Trimethylsilyl Trimethylsiloxy Lactate, Trimethylsilyl Trimethylsiloxy Salicylate, Triphenyl Trimethicone, Trisiloxane, Tris-Tributoxysiloxymethylsilane, Undecylcrylene Dimethicone, Vinyl Dimethicone, Vinyl Dimethicone/Lauryl Dimethicone Crosspolymer, Vinyl Dimethicone/Methicone Silsesquioxane Crosspolymer, Vinyldimethyl/Trimethylsiloxysilicate Stearyl Dimethicone Crosspolymer, VP/Dimethiconylacrylate/Polycarbamyl/Polyglycol Ester, Zinc Carboxydecyl Trisiloxane and Zinc Dimethicone PEG-8 Succinate and mixtures thereof.

More preferably the silicones to be contained in the homecare product composition or cosmetic or pharmaceutical composition according to the invention are Dimethicone, Cyclomethicone, Cyclopentasiloxane, Cyclotetrasiloxane, Phenyl Trimethicone, and Cyclohexasiloxane.

Waxes and/or stabilizers: Besides natural oils used, one or more waxes may also be present in the homecare product composition or cosmetic or pharmaceutical composition according to the present invention, more especially natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Metal salts of fatty acids such as, for example, magnesium, aluminium and/or zinc stearate or ricinoleate may be used as stabilizers.

Primary sun protection factors: In addition to the above disclosed liquid UV filters, the homecare product composition or cosmetic or pharmaceutical composition according to the present invention preferably includes one or more further primary sun protection factors in order to optimize the sun protection factor (SPF) of the formulation, i.e. to obtain a high SPF of 5 to 50, or even 5 to 60, and to cover a broad UVA and UVB range. Primary sun protection factors in the context of the invention are, for example, organic substances (light filters) which are liquid or crystalline at room temperature and which are capable of absorbing ultraviolet radiation and of releasing the energy absorbed in the form of longer-wave radiation, for example heat.

The homecare product composition or cosmetic or pharmaceutical composition according to the invention advantageously contains at least one UV-A filter and/or at least one UV-B filter and/or a broadband filter and/or at least one inorganic pigment. Compositions according to the invention preferably contain at least one UV-B filter or a broadband filter, more particularly preferably at least one UV-A filter and at least one UV-B filter. Preferred cosmetic compositions, preferably topical compositions according to the present invention comprise one, two, three or more sun protection factors selected from the group consisting of 4-aminobenzoic acid and derivatives, salicylic acid derivatives, benzophenone derivatives, dibenzoylmethane derivatives, diphenyl acrylates, 3-imidazol-4-yl acrylic acid and esters thereof, benzofuran derivatives, benzylidene malonate derivatives, polymeric UV-absorbers containing one or more organosilicon radicals, cinnamic acid derivatives, camphor derivatives, trianilino-s-triazine derivatives, 2-hydroxyphenylbenzotriazole derivatives, phenylbenzimidazole sulfonic acid derivatives and salts thereof, anthranilic acid menthyl esters, benzotriazole derivatives and indole derivatives.

In addition, a combination with active ingredients which penetrate into the skin and protect the skin cells from inside against sunlight-induced damage and reduce the level of cutaneous matrix metalloproteases is advantageously. Preferred respective ingredients are so called arylhydrocarbon receptor antagonists. Preferred is 2-benzylidene-5,6-dimethoxy-3,3-dimethylindan-1-one.

The UV-filters cited below which can be used within the context of the present invention are preferred but naturally are not limiting.

UV-filters which are preferably used are selected from the group consisting of
p-aminobenzoic acid
p-aminobenzoic acid ethyl ester (25 mol) ethoxylated (INCI name: PEG-25 PABA)
p-dimethylaminobenzoic acid-2-ethylhexyl ester
p-aminobenzoic acid ethyl ester (2 mol) N-propoxylated
p-aminobenzoic acid glycerol ester
salicylic acid homomenthyl ester (homosalates) (Neo Heliopan®HMS)
salicylic acid-2-ethylhexyl ester (Neo Heliopan®OS)
triethanolamine salicylate
4-isopropyl benzyl salicylate
anthranilic acid menthyl ester (Neo Heliopan®MA)
diisopropyl cinnamic acid ethyl ester
p-methoxycinnamic acid-2-ethylhexyl ester (Neo Heliopan®AV)
diisopropyl cinnamic acid methyl ester
p-methoxycinnamic acid isoamyl ester (Neo Heliopan®E 1000)
p-methoxycinnamic acid diethanolamine salt
p-methoxycinnamic acid isopropyl ester
2-phenylbenzimidazole sulfonic acid and salts (Neo Heliopan®Hydro)
3-(4′-trimethylammonium) benzylidene bornan-2-one methyl sulfate
beta-imidazole-4(5)-acrylic acid (urocanic acid)
3-(4′-sulfo)benzylidene bornan-2-one and salts
3-(4′-methyl benzylidene)-D,L-camphor (Neo Heliopan®MBC)
3-benzylidene-D,L-camphor
N-[(2 and 4)-[2-(oxoborn-3-ylidene) methyl]benzyl] acrylamide polymer
4,4′-[(6-[4-(1,1-dimethyl)aminocarbonyl)phenylamino]-1,3,5-triazine-2,4-diyl)diimino]-bis-(benzoic acid-2-ethylhexyl ester) (Uvasorb® HEB)
benzylidene malonate polysiloxane (Parsol® SLX)
glyceryl ethylhexanoate dimethoxycinnamate
dipropylene glycol salicylate
tris(2-ethylhexyl)-4,4′,4″-(1,3,5-triazine-2,4,6-triyltriimino)tribenzoate (=2,4,6-trianilino-(p-carbo-2′-ethylhexyl-1′-oxy)-1,3,5-triazine) (Uvinul® T150).

In a preferred variant the homecare product composition or cosmetic or pharmaceutical composition according to the present invention comprises a combination with one or more broadband filters which are selected from the group consisting of
2-ethylhexyl-2-cyano-3,3-diphenyl acrylate (Neo Heliopan® 303)
ethyl-2-cyano-3,3′-diphenyl acrylate
2-hydroxy-4-methoxybenzophenone (Neo Heliopan®BB)
2-hydroxy-4-methoxybenzophenone-5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octoxybenzophenone
2-hydroxy-4-methoxy-4′-methyl benzophenone
sodium hydroxymethoxybenzophenone sulfonate
disodium-2,2′-dihydroxy-4,4′-dimethoxy-5,5′-disulfobenzophenone
phenol, 2-(2H-benzotriazol-2-yl)-4-methyl-6-(2-methyl-3(1,3,3,3-tetramethyl-1-(trimethylsilyl)oxy)disiloxyanyl) propyl) (Mexoryl®XL)
2,2′-methylene bis-(6-(2H-benzotriazol-2-yl)-4-1,1,3,3-tetramethylbutyl) phenol) (Tinosorb® M)
2,4-bis-[4-(2-ethylhexyloxy)-2-hydroxyphenyl]-1,3,5-triazine
2,4-bis-[{(4-(2-ethylhexyloxy)-2-hydroxy}phenyl]-6-(4-methoxyphenyl)-1,3,5-triazine (Tinosorb® S)
2,4-bis-[{(4-(3-sulfonato)-2-hydroxypropyloxy)-2-hydroxy}phenyl]-6-(4-methoxyphenyl)-1,3,5-triazine sodium salt
2,4-bis-[{(3-(2-propyloxy)-2-hydroxypropyloxy)-2-hydroxy}phenyl]-6-(4-methoxyphenyl)-1,3,5-triazine
2,4-bis-[{4-(2-ethylhexyloxy)-2-hydroxy}phenyl]-6-[4-(2-methoxyethyl carbonyl) phenylamino]-1,3,5-triazine
2,4-bis-[{4-(3-(2-propyloxy)-2-hydroxypropyloxy)-2-hydroxy}phenyl]-6-[4-(2-ethylcarboxyl) phenylamino]-1,3,5-triazine
2,4-bis-[{4-(2-ethylhexyloxy)-2-hydroxy}phenyl]-6-(1-methylpyrrol-2-yl)-1,3,5-triazine
2,4-bis-[{4-tris-(trimethylsiloxysilylpropyloxy)-2-hydroxy}phenyl]-6-(4-methoxyphenyl)-1,3,5-triazine
2,4-bis-[{4-(2″-methylpropenyloxy)-2-hydroxy}phenyl]-6-(4-methoxyphenyl)-1,3,5-triazine
2,4-bis-[{4-(1′,1′,1′,3′,5′,5′,5′-heptamethylsiloxy-2″-methylpropyloxy)-2-hydroxy}phenyl]-6-(4-methoxyphenyl)-1,3,5-triazine.

In a preferred variant the homecare product composition or cosmetic or pharmaceutical composition according to the present invention comprises a combination with one or more UV-A filters which are selected from the group consisting of
4-isopropyl dibenzoyl methane
terephthalylidene dibornane sulfonic acid and salts (Mexoryl® SX)
4-t-butyl-4′-methoxydibenzoyl methane (avobenzone)/(Neo Heliopan® 357)
phenylene bis-benzimidazyl tetrasulfonic acid disodium salt (Neo Heliopan® AP)
2,2′-(1,4-phenylene)-bis-(1H-benzimidazole-4,6-disulfonic acid), monosodium salt
2-(4-diethylamino-2-hydroxybenzoyl) benzoic acid hexyl ester (Uvinul® A Plus)
indanylidene compounds.

In a more preferred variant, the homecare product composition or cosmetic or pharmaceutical composition according to the present invention comprises a combination with one or more UV filters which are selected from the group consisting of
p-aminobenzoic acid
3-(4′-trimethylammonium) benzylidene bornan-2-one methyl sulfate
salicylic acid homomenthyl ester (Neo Heliopan® HMS)
2-hydroxy-4-methoxybenzophenone (Neo Heliopan® BB)
2-phenylbenzimidazole sulfonic acid (Neo Heliopan® Hydro)
terephthalylidene dibornane sulfonic acid and salts (Mexoryl® SX)
4-tert-butyl-4′-methoxydibenzoyl methane (Neo Heliopan® 357)
3-(4′-sulfo)benzylidene bornan-2-one and salts
2-ethylhexyl-2-cyano-3,3-diphenyl acrylate (Neo Heliopan® 303)
N-[(2 and 4)-[2-(oxoborn-3-ylidene) methyl]benzyl] acrylamide polymer
p-methoxycinnamic acid-2-ethylhexyl ester (Neo Heliopan® AV)
p-aminobenzoic acid ethyl ester (25 mol) ethoxylated (INCI name: PEG-25 PABA)
p-methoxycinnamic acid isoamyl ester (Neo Heliopan® E1000)
2,4,6-trianilino-(p-carbo-2′-ethylhexyl-1′-oxy)-1,3,5-triazine (Uvinul® T150)
phenol, 2-(2H-benzotriazol-2-yl)-4-methyl-6-(2-methyl-3(1,3,3,3-tetramethyl-1-(trimethylsilyl)oxy)disiloxyanyl) propyl) (Mexoryl® XL)
4,4′-[(6-[4-(1,1-dimethyl)aminocarbonyl) phenylamino]-1,3,5-triazine-2,4-diyl)diimino]-bis-(benzoic acid-2-ethylhexyl ester) (Uvasorb HEB)
3-(4′-methyl benzylidene)-D,L-camphor (Neo Heliopan®MBC)
3-benzylidene camphor
salicylic acid-2-ethylhexyl ester (Neo Heliopan® OS)
4-dimethylaminobenzoic acid-2-ethylhexyl ester (Padimate O)
hydroxy-4-methoxybenzophenone-5-sulfonic acid and Na salt
2,2′-methylene bis-(6-(2H-benzotriazol-2-yl)-4-1,1,3,3-tetramethylbutyl) phenol) (Tinosorb® M)
phenylene bis-benzimidazyl tetrasulfonic acid disodium salt (Neo Heliopan®AP)
2,4-bis-[{(4-(2-ethylhexyloxy)-2-hydroxy}phenyl]-6-(4-methoxyphenyl)-1,3,5-triazine (Tinosorb® S)
benzylidene malonate polysiloxane (Parsol® SLX)
menthyl anthranilate (Neo Heliopan® MA)
2-(4-diethylamino-2-hydroxybenzoyl) benzoic acid hexyl ester (Uvinul® A Plus)
indanylidene compounds.

In a further preferred embodiment, the homecare product composition or the cosmetic or pharmaceutical composition according to the invention contains a total amount of sunscreen agents, i.e. in particular UV-filters and/or inorganic pigments (UV filtering pigments) so that the composition according to the invention has a light protection factor of greater than or equal to 2 (preferably greater than or equal to 5). Such compositions according to the invention are particularly suitable for protecting the skin and hair.

Secondary sun protection factors: In addition to the above disclosed liquid UV-filters and besides the groups of primary sun protection factors mentioned above, secondary sun protection factors of the antioxidant type may also be advantageously used in the homecare product composition or cosmetic or pharmaceutical composition according to the present invention. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example alpha-carotene, beta-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, alpha-linoleyl, cholesteryl and glyceryl esters thereof) and their salts, dilaurylthiodipropionate, distearyl-thiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages, also (metal) chelators (for example alpha-hydroxyfatty acids, palmitic acid, phytic acid, lactoferrine), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, titanium dioxide (for example dispersions in ethanol), zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Advantageous inorganic secondary light protection pigments are finely dispersed metal oxides and metal salts. The total quantity of inorganic pigments, in particular hydrophobic inorganic micro-pigments in the finished cosmetic preparation according to the present invention is advantageously from 0.1 to 30% by weight, preferably 0.5 to 10.0% by weight, in each case based on the total weight of the preparation.

Also preferred are particulate UV-filters or inorganic pigments, which can optionally be hydrophobed, can be used, such as the oxides of titanium (TiO₂), zinc (ZnO), iron (Fe₂O₃), zirconium (ZrO₂), silicon (SiO₂), manganese (e.g. MnO), aluminium (Al₂O₃), cerium (e.g. Ce₂O₃) and/or mixtures thereof.

Actives modulating skin and/or hair pigmentation: The cosmetic or pharmaceutical composition according to the present invention may include active ingredients for skin and/or hair lightening. Preferred active ingredients for skin and/or hair lightening are selected from the group consisting of: kojic acid (5-hydroxy-2-hydroxymethyl-4-pyranone), kojic acid derivatives, preferably kojic acid dipalmitate, arbutin, ascorbic acid, ascorbic acid derivatives, preferably magnesium ascorbyl phosphate, hydroquinone, hydroquinone derivatives, resorcinol, resorcinol derivatives, preferably 4-alkylresorcinols and 4-(1-phenylethyl)1,3-dihydroxybenzene (phenylethyl resorcinol), cyclohexylcarbamates, sulfur-containing molecules, preferably glutathione or cysteine, alpha-hydroxy acids (preferably citric acid, lactic acid, malic acid), salts and esters thereof, N-acetyl tyrosine and derivatives, undecenoyl phenylalanine, gluconic acid, chromone derivatives, preferably aloesin, flavonoids, 1-aminoethyl phosphinic acid, thiourea derivatives, ellagic acid, nicotinamide (niacinamide), zinc salts, preferably zinc chloride or zinc gluconate, thujaplicin and derivatives, triterpenes, preferably maslinic acid, sterols, preferably ergosterol, benzofuranones, preferably senkyunolide, vinyl guiacol, ethyl guiacol, dionic acids, preferably octodecene dionic acid and/or azelaic acid, inhibitors of nitrogen oxide synthesis, preferably L-nitroarginine and derivatives thereof, 2,7-dinitroindazole or thiocitrulline, metal chelators (preferably alpha-hydroxy fatty acids, phytic acid, humic acid, bile acid, bile extracts, EDTA, EGTA and derivatives thereof), retinoids, soy milk and extract, serine protease inhibitors or lipoic acid or other synthetic or natural active ingredients for skin and hair lightening, the latter preferably used in the form of an extract from plants, preferably bearberry extract, rice extract, papaya extract, turmeric extract, mulberry extract, bengkoang extract, nutgrass extract, liquorice root extract or constituents concentrated or isolated therefrom, preferably glabridin or licochalcone A, artocarpus extract, extract of rumex and ramulus species, extracts of pine species (pinus), extracts of vitis species or stilbene derivatives isolated or concentrated therefrom, saxifrage extract, scutelleria extract, grape extract and/or microalgae extract, in particular Tetraselmis suecica Extract.

Preferred skin lighteners are kojic acid and phenylethyl resorcinol as tyrosinase inhibitors, beta- and alpha-arbutin, hydroquinone, nicotinamide, dioic acid, Mg ascorbyl phosphate and vitamin C and its derivatives, mulberry extract, Bengkoang extract, papaya extract, turmeric extract, nutgrass extract, licorice extract (containing glycyrrhizin), alpha-hydroxy-acids, 4-alkylresorcinols, 4-hydroxyanisole. These skin lighteners are preferred due to their very good activity, in particular in combination with sclareolide according to the present invention. In addition, said preferred skin lighteners are readily available.

Advantageous skin and hair tanning active ingredients in this respect are substrates or substrate analogues of tyrosinase such as L-tyrosine, N-acetyl tyrosine, L-DOPA or L-dihydroxyphenylalanine, xanthine alkaloids such as caffeine, theobromine and theophyl-line and derivatives thereof, proopiomelanocortin peptides such as ACTH, alpha-MSH, peptide analogues thereof and other substances which bind to the melanocortin receptor, peptides such as Val-Gly-Val-Ala-Pro-Gly, Lys-Ile-Gly-Arg-Lys or Leu-Ile-Gly-Lys, purines, pyrimidines, folic acid, copper salts such as copper gluconate, chloride or pyrrolidonate, 1,3,4-oxadiazole-2-thiols such as 5-pyrazin-2-yl-1,3,4-oxadiazole-2-thiol, curcumin, zinc diglycinate (Zn(Gly)2), manganese(II) bicarbonate complexes (“pseudocat-alases”), tetrasubstituted cyclohexene derivatives, isoprenoids, melanin derivatives such as Melasyn-100 and MelanZe, diacyl glycerols, aliphatic or cyclic diols, psoralens, prostaglandins and analogues thereof, activators of adenylate cyclase and compounds which activate the transfer of melanosomes to keratinocytes such as serine proteases or agonists of the PAR-2 receptor, extracts of plants and plant parts of the chrysanthemum species, san-guisorba species, walnut extracts, urucum extracts, rhubarb extracts, microalgae extracts, in particular Isochrysis galbana, trehalose, erythrulose and dihydroxyacetone. Flavonoids which bring about skin and hair tinting or browning (e.g. quercetin, rhamnetin, kaempferol, fisetin, genistein, daidzein, chrysin and apigenin, epicatechin, diosmin and diosmetin, morin, quercitrin, naringenin, hesperidin, phloridzin and phloretin) can also be used.

Hair growth activators or inhibitors: Cosmetic or pharmaceutical compositions according to the present invention may also comprise one or more hair growth activators, i.e. agents to stimulate hair growth. Hair growth activators are preferably selected from the group consisting of pyrimidine derivatives such as 2,4-diaminopyrimidine-3-oxide (Aminexil), 2,4-diamino-6-piperidinopyrimidine-3-oxide (Minoxidil) and derivatives thereof, 6-amino-1,2-dihydro-1-hydroxy-2-imino-4-piperidinopyrimidine and its derivatives, xanthine alkaloids such as caffeine, theobromine and theophylline and derivatives thereof, quercetin and derivatives, dihydroquercetin (taxifolin) and derivatives, potassium channel openers, antiandrogenic agents, synthetic or natural 5-reductase inhibitors, nicotinic acid esters such as tocopheryl nicotinate, benzyl nicotinate and C1-C6 alkyl nicotinate, proteins such as for example the tripeptide Lys-Pro-Val, diphencypren, hormones, finasteride, dutasteride, flutamide, bicalutamide, pregnane derivatives, progesterone and its derivatives, cyproterone acetate, spironolactone and other diuretics, calcineurin inhibitors such as FK506 (Tacrolimus, Fujimycin) and its derivatives, Cyclosporin A and derivatives thereof, zinc and zinc salts, polyphenols, procyanidins, proanthocyanidins, phytosterols such as for example beta-sitosterol, biotin, eugenol, (±)-beta-citronellol, panthenol, glycogen for example from mussels, extracts from microorganisms, algae, plants and plant parts of for example the genera dandelion (Leontodon or Taraxacum), Orthosiphon, Vitex, Coffea, Paullinia, Theobroma, Asiasarum, Cucurbita or Styphnolobium, Serenoa repens (saw palmetto), Sophora flavescens, Pygeum africanum, Panicum miliaceum, Cimicifuga racemosa, Glycine max, Eugenia caryophyllata, Cotinus coggygria, Hibiscus rosa-sinensis, Camellia sinensis, Ilex paraguariensis, Isochrysis galbana, licorice, grape, apple, barley or hops or/nd hydrolysates from rice or wheat.

Alternatively, the cosmetic or pharmaceutical composition according to the present invention may include one or more hair growth inhibitors (as described above), i.e. agents to reduce or prevent hair growth. Hair growth inhibitors are preferably selected from the group consisting of activin, activin derivatives or activin agonists, ornithine decarboxylase inhibitors such as alpha-difluoromethylornithine or pentacyclic triterpenes like for example ursolic acid, betulin, betulinic acid, oleanolic acid and derivatives thereof, 5alpha-reductase inhibitors, androgen receptor antagonists, S-adenosylmethionine decarboxylase inhibitors, gamma-glutamyl transpeptidase inhibitors, transglutaminase inhibitors, soybean-derived serine protease inhibitors, extracts from microorganisms, algae, different microalgae or plants and plant parts of for example the families Leguminosae, Solanaceae, Graminae, Asclepiadaceae or Cucurbitaceae, the genera Chondrus, Gloiopeltis, Ceramium, Durvillea, Glycine max, Sanguisorba officinalis, Calendula officinalis, Hamamelis virginiana, Arnica montana, Salix alba, Hypericum perforatum or Gymnema sylvestre.

Enzyme inhibitors: The homecare product composition or the cosmetic or pharmaceutical composition according to the present invention may comprise one or more enzyme inhibitors. Suitable enzyme inhibitors are, for example, esterase inhibitors. These are preferably trialkyl citrates, such as trimethyl citrate, tripropyl citrate, triisopropyl citrate, tributyl citrate and, in particular, triethyl citrate (Hydagen CAT). The substances inhibit enzyme activity, thereby reducing the formation of odour. Other substances which are suitable esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

Odour absorbers and/or antiperspirant active agents: The homecare product composition or the cosmetic or pharmaceutical composition according to the present invention may include one or more odour absorbers and/or antiperspirant active agents (antiperspirants). Suitable odour absorbers are substances which are able to absorb and largely retain odour-forming compounds. They lower the partial pressure of the individual components, thus also reducing their rate of diffusion. It is important that perfumes must remain unimpaired in this process. Odour absorbers are not effective against bacteria. They comprise, for example, as main constituent, a complex zinc salt of ricinoleic acid or specific, largely odour-neutral fragrances which are known to the person skilled in the art as “fixatives”, such as, for example, extracts of labdanum or styrax or certain abietic acid derivatives. The odour masking agents are fragrances or perfume oils, which, in addition to their function as odour masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal products, such as, for example, civet and castoreum. Typical synthetic fragrance compounds are products of the ester, ether, aldehyde, ketone, alcohol, and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethole, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden flower oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Suitable astringent antiperspirant active ingredients are primarily salts of aluminium, zirconium or of zinc. Such suitable antihydrotic active ingredients are, for example, aluminium chloride, aluminium chlorohydrate, aluminium dichlorohydrate, aluminium sesquichlorohydrate and complex compounds thereof, e.g. with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, e.g. with amino acids, such as glycine.

Film formers: The homecare product composition or the cosmetic or pharmaceutical composition according to the present invention may include one or more film formers. Standard film formers are preferably chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Carriers and hydrotropes: The homecare product composition or the cosmetic or pharmaceutical composition according to the present invention may comprise a carrier or a mixture of different carriers. Preferred cosmetics carrier materials are solid or liquid at 25° C. and 1013 mbar (including highly viscous substances) as for example glycerol, 1,2-propylene glycol, 1,2-butylene glycol, 1,3-propylene glycol, 1,3-butylene glycol, ethanol, water, and mixtures of two or more of said liquid carrier materials with water. Optionally, these preparations according to the invention may be produced using preservatives or solubilizers. Other preferred liquid carrier substances, which may be a component of a preparation according to the invention are selected from the group consisting of oils such as vegetable oil, neutral oil and mineral oil.

Preferred solid carrier materials, which may be a component of the homecare product composition or cosmetic or pharmaceutical composition according to the invention are hydrocolloids, such as starches, degraded starches, chemically or physically modified starches, dextrins, (powdery) maltodextrins (preferably with a dextrose equivalent value of 5 to 25, preferably of 10-20), lactose, silicon dioxide, glucose, modified celluloses, gum arabic, ghatti gum, traganth, karaya, carrageenan, pullulan, curdlan, xanthan gum, gellan gum, guar flour, carob bean flour, alginates, agar, pectin and inulin and mixtures of two or more of these solids, in particular maltodextrins (preferably with a dextrose equivalent value of 15-20), lactose, silicon dioxide and/or glucose.

In addition, hydrotropes, for example ethanol, isopropyl alcohol or polyols, may be used to improve flow behaviour. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 Dalton;
technical oligoglycerol mixtures with a degree of self-condensation of 1.5 to 10, such as for example technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Dyes: The homecare product composition or the cosmetic or pharmaceutical composition according to the present invention may comprise one or more dyes. Suitable dyes are any of the substances suitable and approved for cosmetic purposes. Examples include cochineal red A (CI. 16255), patent blue V (CI. 42051), indigotin (CI. 73015), chlorophyllin (CI. 75810), quinoline yellow (CI. 47005), titanium dioxide (CI. 77891), indanthrene blue RS (CI. 69800) and madder lake (CI. 58000). Luminol may also be present as a luminescent dye. Advantageous coloured pigments are for example titanium dioxide, mica, iron oxides (e.g. Fe₂O₃ Fe₃O₄, FeO(OH)) and/or tin oxide. Advantageous dyes are for example carmine, Berlin blue, chromium oxide green, ultramarine blue and/or manganese violet.

In addition to the above-described substances, further ingredients commonly used in the homecare product composition or cosmetic or pharmaceutical industry, which are suitable or customary in the compositions of the present invention, can be used.

In a preferred embodiment, the at least one cosmetically or pharmaceutically active substance or active substance customarily used for homecare product compositions and/or additive is selected from the group consisting of agents against ageing of the skin, antioxidants, chelating agents, emulsifiers, preservatives, green and synthetic polymers, rheology additives, oils, fragrances or perfume oils, polyols and mixtures of two or more of the afore-mentioned substances.

In a preferred embodiment, the homecare product composition or the cosmetic or pharmaceutical composition comprises the at least one cosmetically or pharmaceutically active substance or active substance customarily used for homecare product compositions and/or additive is selected from the group consisting of agents against ageing of the skin, antioxidants, chelating agents, emulsifiers, preservatives, green and synthetic polymers, rheology additives, oils, fragrances or perfume oils, polyols and mixtures of two or more of the afore-mentioned substances.

In addition to the above-described substances, further ingredients commonly used in the cosmetic or pharmaceutical industry or for homecare products, which are suitable or customary in the compositions of the present invention, can be used.

Use for Malodour Reduction or Inhibiting Malodour Development or Body Odour Control

The present invention provides for the use of 1,2-nonanediol and/or 2,3-nonanediol, or at least one linear alkanediol or a mixture of at least one first linear alkanediol with one or more second linear alkanediol(s) as defined above for malodour reduction or for inhibiting malodour development or for body odour control or for masking insect alluring odours.

The 1,2-nonanediol or the 2,3-nonanediol or the mixture comprising 1,2-nonanediol and 2,3-nonanediol is present in the composition according to the first aspect of the present invention or in the ready to use product, i.e., in the final cosmetic or pharmaceutical preparation or homecare product, in an amount of 0.001 to 15.0% by weight, based on the total weight of the composition or final cosmetic or pharmaceutical preparation or homecare product. In a preferred variant, the composition or the final preparation comprises the 1,2-nonanediol or the 2,3-nonanediol or the mixture comprising 1,2-nonanediol and 2,3-nonanediol in an amount of 0.005 to 10.0% by weight, based on the total weight of the composition or final cosmetic or pharmaceutical preparation or homecare product. In a particular preferred variant, the 1,2-nonanediol or the 2,3-nonanediol or the mixture comprising 1,2-nonanediol and 2,3-nonanediol is advantageously used in the composition or final preparation in an amount of 0.01 to 5.0% by weight, based on the total weight of the composition or final cosmetic or pharmaceutical preparation or homecare product. In a most preferred variant, the 1,2-nonanediol or the 2,3-nonanediol or the mixture comprising 1,2-nonanediol and 2,3-nonanediol is advantageously used in the composition or final preparation in an amount of 0.02 to 3.0% by weight, based on the total weight of the composition or final cosmetic or pharmaceutical preparation or homecare product.

For the nonanediol-mixture, the above amounts relate to the total content of the alkanediols 1,2-nonanediol and the 2,3-nonanediol in the mixture, i.e. the amount is the sum of the content of the alkanediols 1,2-nonanediol and 2,3-nonanediol in the mixture.

The at least one linear alkanediol or the mixture comprising at least one first linear alkanediol and one or more second linear alkanediol(s) according to the second aspect of the present invention is present in the composition in an amount of 0.001 to 15.0% by weight, based on the total weight of the composition or final cosmetic or pharmaceutical preparation or homecare product. In a preferred variant, the composition comprises the at least one linear alkanediol or the mixture comprising at least one first linear alkanediol and one or more second linear alkanediols in an amount of 0.005 to 10.0% by weight, based on the total weight of the composition or final cosmetic or pharmaceutical preparation or homecare product. In a particular preferred variant, the at least one linear alkanediol or the mixture comprising at least one first linear alkanediol and one or more second linear alkanediols is advantageously used in the composition in an amount of at 0.01 to 5.0% by weight, based on the total weight of the composition or final cosmetic or pharmaceutical preparation or homecare product. In a most preferred variant, the at least one linear alkanediol or the mixture comprising at least one first linear alkanediol and one or more second linear alkanediols is advantageously used in the cosmetic or pharmaceutical composition or homecare product in an amount of at 0.02 to 3.0% by weight, based on the total weight of the composition or final cosmetic or pharmaceutical preparation or homecare product.

For the alkanediol-mixture, the above amounts refer to the total content of the first linear alkanediol(s) and the second linear alkanediol(s) in the mixture, i.e. the amount is the sum of the content of the first linear alkanediol(s) and the second linear alkanediol(s) in the mixture.

In a particular preferred variant according to the first aspect of the present invention, the composition comprises the 2,3-nonanediol or the 2,3-nonanediol or the mixture comprising 1,2-nonanediol and 2,3-nonanediol in an amount of 0.001 to 15.0% by weight, preferred in an amount of 0.005 to 10.0% by weight, more preferred in an amount of 0.01 to 5.0% by weight, and most preferred in an amount of 0.02 to 3.0% by weight, based on the total weight of the cosmetic or pharmaceutical composition or homecare product composition or final cosmetic or pharmaceutical preparation or homecare product.

Even more preferred, the cosmetic or pharmaceutical composition or the homecare product composition comprises the 2,3-nonanediol or the 2,3-nonanediol or the mixture comprising 1,2-nonanediol and 2,3-nonanediol in an amount of 0.001 to 0.5% weight, preferably in an amount of 0.005 to 0.1% by weight and most preferably in an amount of 0.01 to 0.075% by weight, based on the total weight of the composition or final cosmetic or pharmaceutical preparation or homecare product.

In a particular preferred variant according to the first aspect of the present invention, the composition comprises the 2,3-alkandediol as linear alkanediol or the mixture comprising at least one first linear alkanediol and one or more second linear alkanediol(s) in an amount of 0.001 to 15.0% by weight, preferably in an amount of 0.005 to 10.0% by weight, more preferred in an amount of 0.01 to 5.0% by weight, and most preferred in an amount of 0.02 to 3.0% by weight, based on the total weight of the composition or final cosmetic or pharmaceutical preparation or homecare product.

Even more preferred, the composition comprises the 2,3-alkanediol as linear alkanediol or the mixture comprising at least one first linear alkanediol and one or more second linear alkanediol(s) in an amount of 0.001 to 0.5% weight, preferably in an amount of 0.005 to 0.1% by weight and most preferably in an amount of 0.01 to 0.075% by weight, based on the total weight of the composition or final cosmetic or pharmaceutical preparation or homecare product.

Cosmetic Product

The present invention provides a cosmetic product comprising a cosmetic composition according to the present invention, wherein the product is a deodorant and/or antiperspirant, an aerosol-based deo spray, deo pump spray, deo stick, deo roll on, deo cream, deo wipes, deo crystals, pickering emulsions, hydrodispersion gels, skin balms, shampoo, shower gel, foam bath, micellar water, facial cleansing solutions, cleansing wipes, intimate spray, intimate cream, intimate wash lotion, intimate wipes, aerosol-based foot spray, foot pump spray, foot bath, foot balm, soap, liquid washing, shower and bath preparation, bath product (capsule, oil, tablet, salt, bath salt, soap, etc.), effervescent preparation, mouth wash concentrate, mouth wash ready to use, repellent, and anti-insect applications, in particular insect repellent lotion, spray, solution or cream, mosquito repellent, human-smell masking application, perfume composition and tooth paste.

The present invention provides for the use of 1,2-nonanediol and/or 2,3-nonanediol, or at least one linear alkanediol or a mixture of at least one first linear alkanediol with one or more second linear alkanediol(s) as defined above for producing a cosmetic product or a homecare product according to the present invention.

Moreover, the present invention provides for the use of 1,2-nonanediol, 2,3-nonanediol, or a mixture of 1,2-nonanediol with 2,3-nonanediol, or at least one linear alkanediol or a mixture of at least one first linear alkanediol with one or more second linear alkanediol(s) as defined herein for malodour reduction or for inhibiting malodour development or for body odour control or for masking insect alluring odours.

The cooling agent is present in the composition according to the present invention or the final cosmetic or pharmaceutical preparation or homecare product in an amount of 0.0001 to 10.0% by weight, based on the total weight of the composition or final cosmetic or pharmaceutical preparation or homecare product. In a preferred variant, the composition or the final cosmetic or pharmaceutical preparation or homecare product comprises the cooling agent in an amount of 0.001 to 5.0% by weight, based on the total weight of the composition or final cosmetic or pharmaceutical preparation or homecare product. In a further particular preferred variant, the cooling agent is advantageously used in the composition or in the final cosmetic or pharmaceutical preparation or homecare product in an amount of 0.005 to 4.0% by weight, based on the total weight of the composition or final cosmetic or pharmaceutical preparation or homecare product, and even more preferred in an amount of 0.01 to 3.0% by weight, based on the total weight of the composition or final cosmetic or pharmaceutical preparation or homecare product.

The present invention shall now be described in detail with reference to the following examples, which are merely illustrative of the present invention, such that the content of the present invention is not limited by or to the following examples.

As demonstrated in the following examples, compositions comprising 1,2-nonanediol and/or 2,3-nonanediol, or at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different from the first linear alkanediol, in combination with at least one cooling agent provide an excellent cooling effect, an excellent antimicrobial effect and an excellent malodour coverage effect. Therefore, such compositions are highly suitable for the preparation of cosmetic or pharmaceutical preparations, or the homecare products based on a pronounced synergistic effect of the alkanediols as such in mixture as well as the alkanediols in combination with cooling agents, allowing for an efficient inhibition of the development of an unpleasant smell due to the excellent antimicrobial effect, the efficient coverage of unpleasant odours as well as insect alluring odours. Consequently, the homecare product compositions as well as the cosmetic or pharmaceutical compositions according to the present invention are highly efficient in malodour reduction, in inhibiting malodour development, in body odour control and in masking insect alluring odours, respectively. Finally, the cosmetic or pharmaceutical compositions and the homecare product compositions according to the present invention are toxicologically acceptable, readily tolerated by the skin, stable, inexpensive to prepare, easy to formulate, active at different pH-values and do not possess an unpleasant smell.

The homecare products according to the first and second aspect of the invention are predominantly detergent formulations, usually liquids, powders, sprays, granules or tablets, used to remove dirt, including dust, stains, bad smells and clutter on surfaces or other types of housecleaning or laundry detergent that is added for cleaning laundry or liquid soap. Typical homecare products include all-purpose cleaners, dishwashing detergents, hard floor and surface cleaners, glass cleaners, carpet cleaners, oven cleaners, laundry detergents, fabric softeners, laundry scent, scent lotions, car shampoo, rim block gel, all afore-mentioned goods also in encapsulated form, air fresheners or furniture polish.

Due to their outstanding antimicrobial efficacy and odour improving properties as described above, the composition according to the first and second aspect of the present invention is suitable for the preparation of cosmetic or pharmaceutical preparations or homecare products.

Additionally, the compositions comprising or consisting of
1,2-nonanediol and/or 2,3-nonanediol;
at least one cooling agent;
optionally at least one antimicrobial compound; and
optionally at least one active substance for a cosmetic or pharmaceutical composition or a homecare product composition and/or additive;
   or
   compositions, comprising or consisting of
at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol;
at least one cooling agent;
optionally at least one antimicrobial compound; and
optionally at least one active substance for a cosmetic or pharmaceutical composition or a homecare product composition and/or additive
   can advantageously be incorporated into a variety of further consumer products. Such products include but are not limited to diapers, bibs, wipes; car care products, dishwashing, fabric conditioning (including softening), laundry detergency, laundry and rinse additive and/or care, hard surface cleaning and/or treatment, paper handkerchiefs, and/or paper towels; tampons, sanitary napkins and the like.

As used herein “consumer products” are defined as a functionally effective combination of ingredients which are consumed i.e. used up, in performing the task for which the product was intended such as cleansing, cleaning, deodorising, sanitising, softening, freshening, etc. Said products are for example, personal care products (shampoo, hair conditioner, shower gels, body was, liquid or solid soap, deodorant sprays, roll-on and stick deodorants, moist wipes such as baby wipes or moist toilet tissues, refreshing wipes etc.), laundry detergents and conditioners (liquid and powdered detergents, tumble dryer sheets, fabric softener etc.), household cleaners (surface cleaners, floor cleaner, dishwashing liquid, powder or tablets, bathroom and sanitary cleaners, fabric cleaners, carpet cleaners etc.), air care products (liquid electric powered air freshener devices, aerosol sprays, pump action sprays, fragranced candles, membrane permeation devices, Liquid wick devices, oil based gel fragrances, aqueous gels etc.).

### EXAMPLES

### A) Antimicrobial Activity: Minimum Inhibitory Concentration (MIC)

Methods

The MIC test is a test on growth inhibition. Estimation of the Minimum Inhibitory Concentration (MIC) is executed in 96 well plates. Through the comparison of bacterial growth with positive and negative controls via optical density (OD), different concentrations of given test substances are evaluated. Bacteria are cultivated under adjusted conditions based on information of the German Collection of Microorganisms and Cell Cultures (DSMZ) and stored in 50% Glycerol prior to use. Afterwards, the following method was applied:

Adding growth medium (according to microorganism) to each well of the microplate. Adding the test-substances in different concentrations, positive controls (water controls only with medium) and negative controls (benchmarks according to organism). Adding the microorganism in a concentration of 106 CFU/ml (CFU=colony forming units). Incubation of the microplates at appropriate conditions.

According to resulting growth, substance concentrations are labelled either as inhibiting or not and the corresponding MIC is defined as the lowest concentration where a complete growth inhibition is visible. Experiments at concentration limits (highest concentration labelled as growth and lowest concentration labelled as inhibiting) are performed at least twice.

### Results

### Example 1: Antimicrobial Properties of Alkanediols

TABLE 1
Antimicrobial properties of 1,2-heptanediol (1,2-C7), 1,2-
nonanediol (1,2-C9), 1,2-undecandiol (1,2-C11), 2,3-nonanediol (2,3-C9) and
2,3-undecanediol (2,3-C11) against microorganisms naturally related to the
human skin: Minimum Inhibitory Concentration (MIC) in ppm.

| | Staphylo- | | | Staphylo- | Coryne- | Propioni- |
|---|---|---|---|---|---|---|
| | coccus | Candida | Escherichia | coccus | bacterium | bacterium |
| | aureus | albicans | coli | hominis | jeikeium | acnes |
| 1,2-C7 | 10 000 | 5 000 | 5 000 | 10 000 | 7 500 | >5 000 |
| 1,2-C9 | 1 000 | 1 000 | 500 | 1 000 | 2 500 | 1 000 |
| 1,2-C11 | 125 | 64 | 250 | 125 | 250 | 250 |
| 2,3-C9 | 2 500 | 2 500 | 1 000 | 5 000 | 2 500 | 2 500 |
| 2,3-C11 | 250 | 250 | 500 | 250 | 250 | 250 |

Minimum Inhibitory Concentrations (MIC) are given in ppm. “>5000” indicates that 5000 ppm was tested, and no growth inhibition found, higher values were not investigated.

As can be seen from these results, all the tested alkanediols exhibited an excellent antimicrobial effect for the tested microorganisms naturally related to the human skin. The best antimicrobial effect was achieved by 1,2-undecanediol and 2,3-undecanediol. In addition, 1,2-nonanediol and 2,3-nonanediol exhibited a remarkably strong antimicrobial effect. It was only recently discovered that the microorganism Corynebacterium jeikeium is one microorganism which is responsible for producing compounds having an unpleasant smell from human sweat. The inhibition of the microbial activity of Corynebacterium jeikeium is difficult. It has been surprisingly found, that all of the tested alkanediols exhibited an antimicrobial effect for Corynebacterium jeikeium. Especially, the tested nonanediols and undecanediols showed a remarkably strong antimicrobial effect for Corynebacterium jeikeium. Therefore, all the tested alkanediols are especially useful for malodour reduction, inhibiting malodour development and body odour control, in particular 1,2-nonanediol, 2,3-nonanediol, 1,2-undecanediol and 2,3-undecanediol.

### Example 2: Further Antimicrobial Properties of 1,2-Nonanediol

TABLE 2
Antimicrobial properties of 1,2-nonanediol (1,2-C9): Minimum
Inhibitory Concentration (MIC) in ppm.

| Organism | MIC (ppm) |
|---|---|
| Aspergillus niger | 2 500 |
| Corynebacterium xerosis | 2 500 |
| Staphylococcus epidermidis | 1 000 |
| Anaerococcus octavius | 1 000 |

In addition, as can be seen from the results of Table 2, 1,2-nonanediol exhibited an excellent antimicrobial effect for further tested microorganisms naturally related to the human skin. It was only recently discovered that the anaerobic microorganism Anaerococcus octavius is one microorganism which is responsible for producing compounds having an unpleasant smell from human sweat. The inhibition of the microbial activity of Anaerococcus octavius is difficult. It has been surprisingly found, that 1,2-nonanediol exhibited an antimicrobial effect for Anaerococcus octavius. Consequently, 1,2-nonanediol is especially useful for malodour reduction, inhibiting malodour development and body odour control.

### B) Example on the Synergistic Activity of 1,2-Alkanediols with Cooling Compounds

Methods

The synergistic activity of 1,2-alkanediols with cooling compounds was determined by measuring the corresponding minimum inhibitory concentrations (MIC values) according to the method described above for antimicrobial activity: Minimum Inhibitory Concentration (MIC)

In order to calculate the synergy index with MIC values, the Kull equation (I) was used as follows:

SI=(MICₘᵢₓ×P_{A})/MIC_{A}+(MICₘᵢₓ×P_{B})/MIC_{B} (I)

wherein
SI is the Synergy Index according to Kull
MIC_{A} is the MIC value for Substance A
MIC_{B} is the MIC value for Substance B
MICₘᵢₓ is the MIC value for the mixture of substances A and B
P_{A} is the proportion of the substance A in the mixture
P_{B} is the proportion of the substance B in the mixture

### Results

### Example 3: Synergistic Activity of Alkanediols and Other Cooling Agents

To verify that the tested combinations act synergistically against microorganisms of the natural microbiota of mammalian skin, a corresponding experiment was conducted with representative microbial species. The synergistic activities of mixtures of 1,2-heptanediol and Menthyl lactate as well as for 1,2-nonanediol and Menthyl lactate were determined by measuring the corresponding minimum inhibitory concentrations (MIC values) as indicated in the methods section.

For all two microorganisms, synergistic effects regarding the antimicrobial efficacy were found with SI values below 1.

TABLE 3
Minimum inhibitory concentrations and resulting
Synergy Indices SI of 1,2-heptanediol (1,2-C7)
and 1,2-nonanediol (1,2-C9) with menthyl lactate.

| | MIC (ppm) | | Synergy index SI | |
|---|---|---|---|---|
| | Staphylo- | Coryne- | Staphylo- | Coryne- |
| | coccus | bacterium | coccus | bacterium |
| | hominis | xerosis | hominis | xerosis |
| Menthyl lactate | 250 | 250 | | |
| 1,2-C7 | 10 000 | 5 000 | | |
| 1,2-C9 | 1 000 | 2 500 | | |
| Menthyl lactate and | 250 | 250 | 0.81 | 0.81 |
| 1,2-C7 in a mixture | | | | |
| of 80%:20% | | | | |
| Menthyl lactate and | 250 | 250 | 0.85 | 0.82 |
| 1,2-C9 in a mixture | | | | |
| of 50%:50% | | | | |

As can be seen from these results, the combination of 1,2-heptanediol and/or 1,2-nonanediol with Frescolat ML is especially useful for malodour reduction, inhibiting malodour development and body odour control based on a synergistic antimicrobial effect. It is believed that generally, the combination of alkanediols according to the present invention with at least one cooling agent according to the present invention exhibits a synergistic antimicrobial effect. Therefore, the cosmetic and pharmaceutical compositions as well as the homecare product compositions according to the present invention are especially useful for malodour reduction, inhibiting malodour development and body odour control, respectively, based on a strongly pronounced synergistic antimicrobial effect.

TABLE 4
Minimum inhibitory concentrations and resulting Synergy Indices
SI of heptanediol (C7) and nonanediol (C9) with further cooling
agents (in view of Corynebacterium xerosis).

| | MIC (ppm) | Synergy index SI |
|---|---|---|
| | Corynebacterium xerosis | |
| Menthol | 1 000 | |
| 3-((−)-Menthoxy)propane-1,2-diol | 500 | |
| (Coolact 10, Cooling Agent 10) | | |
| 1,2-C7 | 5 000 | |
| 1,2-C9 | 2 500 | |
| 2,3-C7 | 12 500 | |
| 2,3-C9 | 2 500 | |
| 1,2-C9 and menthol | 1 000 | 0.7 |
| in a mixture of 50%:50% | | |
| 1,2-C9 and 3-(−)-Menthoxy)propane- | 500 | 0.6 |
| 1,2-diol | | |
| in a mixture of 50%:50% | | |
| Mixture of 1,2-C7 with 2,3-C7 and | 1 000 | 0.6 |
| menthol | | |
| (45%:5%:50%) | | |
| Mixture of 1,2-C9 with 2,3-C9 and | 1 000 | 0.7 |
| menthol | | |
| (45%:5%:50%) | | |

As can be seen from these results in Table 4, the combination of 1,2-heptanediol and/or 2,3-heptanediol and 1,2-nonanediol and/or 2,3-nonanediol with the cooling agents menthol and 3-((−)-menthoxy)propane-1,2-diol is especially useful for efficient malodour reduction, inhibition of malodour development and body odour control based on a pronounced synergistic antimicrobial effect arising between the alkanediol(s) and the cooling agent.

### C) Malodour Coverage and Masking Insect Alluring Odours

Method

Malodour coverage was tested in an established setup with a trained panel (n=5-9). For this sensory test, a defined amount of the material was applied on a filter paper together with a defined amount of sweat malodour. For evaluation, the panelists were asked to rate the individual samples on a scale from 0 to 10 (0: odourless; 10: strongest imaginable).

### Results

### Example 4: Synergistic Malodour Coverage Effect of Alkanediols and a Cooling Agent

The results of the malodour rating for all materials were ranked and the position of the individual materials compared over time. A synergistic effect regarding reduction of malodour was found for all mixtures of Frescolat ML and 1,2-heptanediol after 24 h and 48 h, respectively.

TABLE 5
Results of the malodour coverage experiments for the test
compositions based on 1,2-heptanediol in combination
with a cooling agent after 24 h and 48 h

| | 24 h | 48 h |
|---|---|---|
| Frescolat ML | 3.6 | 3.1 |
| 1,2-heptanediol | 3.3 | 3.3 |
| 50% Frescolat ML + | 3.3 | 2.5 |
| 50% 1,2-heptanediol | | |
| 80% Frescolat ML + | 2.4 | 3.1 |
| 20% 1,2-heptanediol | | |
| 20% Frescolat ML + | 2.5 | 2.9 |
| 80% 1,2-heptanediol | | |

(Ranking: 1 = lowest malodour to 6 = strongest malodour).

As can be seen from the results above the pure compounds Frescolat ML, a colourless liquid used as an active cooling agent, and 1,2-heptanediol exhibit an odour with a rate of 3.6 and 3.3 after 24 h. Consequently, corresponding mixtures of both compounds should exhibit an expected odour with a rate ranging between 3.3 and 3.6 measured after 24 h. However, actually the mixtures of both compounds in different ratios exhibit an odour with a rate between 2.4 and 3.3 after 24 h. Therefore, the mixtures of both compounds, i.e. compositions comprising an alkanediol according to the present invention and a typical cooling agent, show a synergistic malodour coverage effect after 24 h.

In addition, the results shown above indicate that the pure compounds Frescolat ML and 1,2-heptanediol exhibit an odour with a rate of 3.1 and 3.3 after 48 h. Consequently, corresponding mixtures of both compounds should exhibit an expected odour with a rate ranging between 3.1 and 3.3 measured after 48 h. However, actually the mixtures of both compounds in different ratios exhibit an odour with a rate between 2.5 and 3.1 after 48 h. Therefore, the mixtures of both compounds, i.e., the cooling agent and the 1,2-alkanediol, show a synergistic malodour coverage effect after 48 h.

These results demonstrate that the combination of 1,2-heptanediol with Frescolat ML is especially useful for malodour reduction, inhibiting malodour development and body odour control based on a synergistic malodour coverage effect. It is believed that generally, the combination of alkanediols according to the present invention with at least one cooling agent according to the present invention exhibits an advantageous synergistic malodour coverage effect. Therefore, the cosmetic and pharmaceutical compositions as well as the homecare product compositions according to the present invention are especially useful for malodour reduction, inhibiting malodour development and body odour control, respectively, based on a pronounced synergistic malodour coverage effect.

TABLE 6
Results of the malodour coverage experiments for the test compositions
based on nonanediol in combination with a cooling agent after 6
h and 24 h

| | 6 h | 24 h |
|---|---|---|
| Frescolat ML | 3.6 | 3.9 |
| 1,2-Nonanediol | 4.2 | 3.9 |
| 50% Frescolat ML + | 3 | 2.7 |
| 50% 1,2-nonanediol | | |
| 80% Frescolat ML + | 4 | 3.6 |
| 20% 1,2-nonanediol | | |
| 20% Frescolat ML + | 3 | 3.9 |
| 80% 1,2-nonanediol | | |

(Ranking: 1 = lowest malodour to 6 = strongest malodour).

Table 6 shows a pronounced synergistic effect of the combination of 1,2-nonanediol with a cooling agent: The combination of 50% Frescolat ML with 50% 1,2-nonanediol showed an efficient improvement of odour, i.e. a pronounced malodour reduction after 6 h as well as after 24 h. Furthermore, a good malodour reduction effect was observed for a mixture of 80% Frescolat ML with 20% 1,2-nonanediol after 24 h and for a mixture of 20% Frescolat ML with 80% 1,2-nonanediol after 6 h, respectively, based on a synergism between the alkanediol and the cooling agent.

TABLE 7
Results of the malodour coverage experiments for the test
compositions based on heptanediol (C7) after 6 h and 24 h

| | 6 h | 24 h |
|---|---|---|
| 0.5% 1,2-C7 | 2.3 | 3.4 |
| 0.5% 2,3-C7 | 3.2 | 3.5 |
| Mixture of 1,2-C7 and 2,3-C7 | 3.4 | 4 |
| in a ratio of 95:5 | | |
| Mixture of 1,2-C7 and 2,3-C7 | 3.4 | 4 |
| in a ratio of 7:3 | | |

(Ranking: 1 = highest malodour to 6 = lowest malodour).

Table 7 shows the results of the malodour coverage experiments for samples being based on heptanediols (C7): The above results show that a combination of 1,2-heptanediol with 2,3-heptanediol in different ratios allows for a pronounced improvement in odour based on an efficient malodour coverage compared to the use of 1,2-heptanediol of 2,3-heptanediol alone. A combination of the isomeric heptanediols results in a synergistic efficiency with is superior to the effects of the single substances. A further combination of said isomeric mixtures with additional cooling agents results in an even more increased synergy. Thereby, already small portions of the 2,3-compound are sufficient in order to achieve a pronounced advantageous effect.

TABLE 8
Results of the malodour coverage experiments for the test
compositions based on octanediol (C8) after 6 h and 24 h

| | 24 h |
|---|---|
| 0.5% 1,2-C8 | 2.8 |
| 0.5% 2,3-C8 | 3.2 |
| Mixture of 1,2-C8 and 2,3-C8 | 3.6 |
| in a ratio of 95:5 | |
| Mixture of 1,2-C8 and 2,3-C8 | 3.7 |
| in a ratio of 1:1 | |
| Mixture of 1,2-C8 and 2,3-C8 | 4.2 |
| in a ratio of 7:3 | |

(Ranking: 1 = highest malodour to 6 = lowest malodour).

As can be seen from the results above the pure alkanediol compounds exhibit an odour with a rate of 2.8 and 3.2 after 24 h. Consequently, corresponding mixtures of both compounds should exhibit an expected odour with a rate ranging between 2.8 and 3.2 measured after 24 h. However, actually the mixtures of both compounds in different ratios exhibit an odour with a rate ranging between 3.6 and 4.2 after 24 h indicating an advantageous synergy between the isomeric octanediol compounds. Therefore, the mixtures of both isomeric forms, i.e. compositions comprising an 1,2-alkanediol and an 2,3-alkanediol according to the present invention, show a synergistic malodour coverage effect after 24 h. Thereby, already small portions of the 2,3-compound are sufficient in order to achieve a pronounced advantageous effect.

TABLE 9
Results of the malodour coverage experiments for the test
compositions based on combinations of heptanediols
(C7) and octanediols (C8) after 6 h and 24 h

| | 6 h | 24 h |
|---|---|---|
| 0.5% 1,2-C7 | 3.5 | 3.1 |
| 0.5% 2,3-C8 | 2.3 | 3.4 |
| Mixture of 1,2-C7 and 2,3-C8 | 3.8 | 3.8 |
| in a ratio of 95:5 | | |
| Mixture of 1,2-C7 and 2,3-C8 | 3.5 | 4.1 |
| in a ratio of 1:1 | | |
| Mixture of 1,2-C7 and 2,3-C8 | 4.5 | 3.4 |
| in a ratio of 7:3 | | |

(Ranking: 1 = highest malodour to 6 = lowest malodour).

As can be seen from the results above the pure alkanediol compounds differing in their chain lengths exhibit an odour with a rate of 3.1 and 3.4 after 24 h. Consequently, corresponding mixtures of both compounds should exhibit an expected odour with a rate ranging between 3.2 and 3.4 measured after 24 h. However, actually the mixtures of both compounds in different ratios exhibit an odour with a rate ranging between 3.4 and 4.1 after 24 h indicating an advantageous synergy between the 1,2-heptanediol and the 2,3-octanediol having different chain lengths. Therefore, the mixtures of said different alkanediols in different ratios, i.e. compositions comprising an 1,2-alkanediol and an 2,3-alkanediol differing in their chain lengths (hetero combination) according to the present invention, show a synergistic malodour coverage effect after 24 h. Thereby, already small portions of the 2,3-compound are sufficient in order to achieve a pronounced advantageous effect and to efficiently cover unpleasant malodour.

### Example 5: Malodour Coverage Effect on Fragrance Ingredients

Method

Testing Conditions:

The fragrance ingredients (1% w/w) in aqueous/ethanolic solution (7:3) were sprayed on paper stripes and compared with solutions containing alkanediols after 10 min and 25 min, respectively.

Tested Alkanediols:

TABLE 10
Tested alkanediols.

| Alkanediol | Abbreviation |
|---|---|
| 1,2-Heptanediol | =1,2-C7 |
| 2,3-Heptanediol | =2,3-C7 |
| 1,2-Octanediol | =1,2-C8 |
| 2,3-Octanediol | =2,3-C8 |

TABLE 11
Tested fragrance blend used in Example 5.

| Name | Structure | w/w % |
|---|---|---|
| Aldehyd C12 NMA | 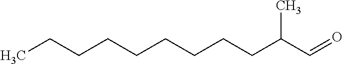 | 25.0 |
| Globalide | 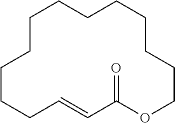 [SMILES: O=C1/C=C/CCCCCCCCCCCCO1] | 25.0 |
| Dihydro- myrcenol | 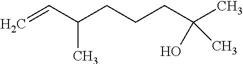 | 25.0 |
| Citronellyl- acetat | 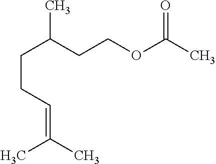 | 25.0 |

Preparation of the Fragrance Blend (“FRA-Blend”):

The fragrance ingredients specified above (Table 11) were mixed by stirring at ambient temperature with a magnetic stirrer for 5 min at 350 rpm.

TABLE 12
Tested ethanolic/aqueous solutions (C7 = heptanediol; C8 = octanediol).

| | w/w % | | | | | | |
|---|---|---|---|---|---|---|---|
| | Samples | Sample | Sample | Sample | Sample | Sample | Sample |
| | 1 & 5 | 2 | 3 | 4 | 6 | 7 | 8 |
| Substance | (placebo) | heptanediol | | | octanediol | | |
| Ethanol pa. | 69.0 | 67.0 | 67.0 | 67.0 | 67.0 | 67.0 | 67.0 |
| Water, dist. | 30.0 | 30.0 | 30.0 | 30.0 | 30.0 | 30.0 | 30.0 |
| FRA-Blend | 1.0 | 1.0 | 1.0 | 1.0 | 1.0 | 1.0 | 1.0 |
| 1,2-C7 | — | 2.0 | — | 1.0 | — | — | — |
| 2,3-C7 | — | — | 2.0 | 1.0 | — | — | — |
| 1,2-C8 | — | — | — | — | 2.0 | — | 1.0 |
| 2,3-C8 | — | — | — | — | — | 2.0 | 1.0 |

Preparation of the Fragrance-Solutions:

The FRA-Blend specified above was pre-solved in the alkanediol (mixture) according to Table 12 and ethanol added. The solution was vortexed for 15 seconds. Subsequently, water was added and the resulting solution vortexed again for further 15 seconds.

For evaluating the sensorial behaviour of alkanediols in combination with different odorous compounds, the odour compounds were blended with the alkanediol (mixture) and dissolved in a EtOH/water-solution and assessed by untrained panelists (P1, P2, P3, P4, P5) in comparison to the corresponding samples which were prepared without the addition of alkanediol(s) (placebo sample). The odour intensity was accorded a corresponding value between 1 and 5 (Ranking: 1=weak odour; 5=strong odour/high odour intensity).

Results

TABLE 13
Evaluation after 10 minutes (alkanediol: heptanediol (C7)).

| | Sample 1 | Sample 2 | Sample 3 | Sample 4 |
|---|---|---|---|---|
| Panelist | (placebo) | (1,2-C7) | (2,3-C7) | (1,2-C7 & 2,3-C7) |
| P1 | 5 | 2 | 3 | 2 |
| P2 | 5 | 3 | 2 | 1 |
| P3 | 5 | 2 | 3 | 3 |
| P4 | 5 | 3 | 4 | 3 |
| P5 | 5 | 2 | 3 | 2 |
| Mean value Ø | 5.0 | 2.4 | 3.0 | 2.2 |

TABLE 14
Evaluation after 25 minutes (alkanediol: heptanediol (C7)).

| | Sample 1 | Sample 2 | Sample 3 | Sample 4 |
|---|---|---|---|---|
| Panelist | (placebo) | (1,2-C7) | (2,3-C7) | (1,2-C7 & 2,3-C7) |
| P1 | 4 | 2 | 2 | 1 |
| P2 | 5 | 3 | 2 | 1 |
| P3 | 5 | 3 | 2 | 2 |
| P4 | 5 | 2 | 4 | 2 |
| P5 | 4 | 2 | 2 | 1 |
| Mean value Ø | 4.6 | 2.4 | 2.4 | 1.4 |

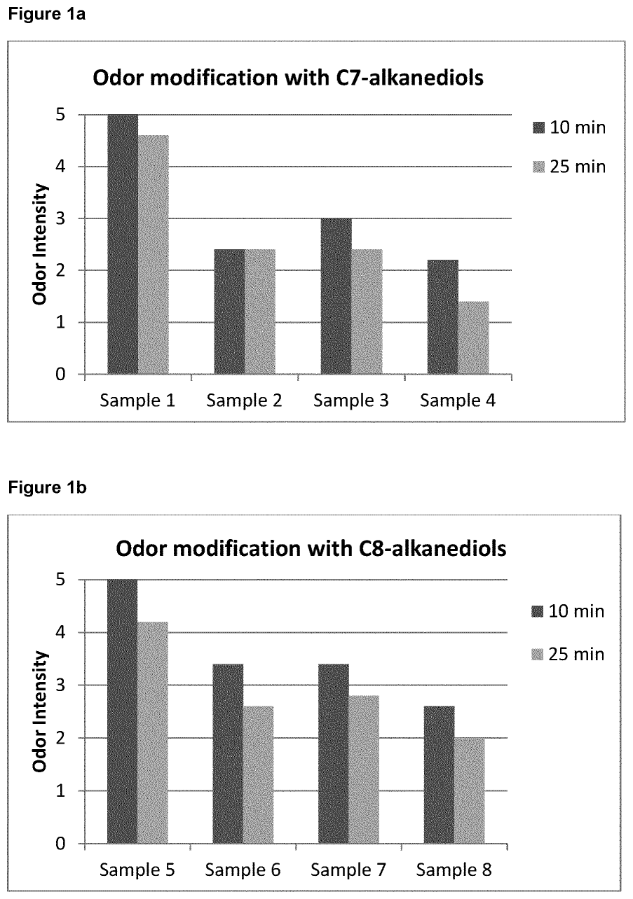

The results of the evaluation are additionally shown in FIG. 1a.

TABLE 15
Evaluation after 10 minutes (alkanediol: octanediol (C8)).

| | Sample 5 | Sample 6 | Sample 7 | Sample 8 |
|---|---|---|---|---|
| Panelist | (placebo) | (1,2-C8) | (2,3-C8) | (1,2-C8 & 2,3-C8) |
| P1 | 5 | 3 | 3 | 2 |
| P2 | 5 | 4 | 4 | 3 |
| P3 | 5 | 5 | 4 | 3 |
| P4 | 5 | 2 | 3 | 3 |
| P5 | 5 | 3 | 3 | 2 |
| Mean value Ø | 5.0 | 3.4 | 3.4 | 2.6 |

TABLE 16
Evaluation after 25 minutes (alkanediol: octanediol (C8)).

| | Sample 5 | Sample 6 | Sample 7 | Sample 8 |
|---|---|---|---|---|
| Panelist | (placebo) | (1,2-C8) | (2,3-C8) | (1,2-C8 & 2,3-C8) |
| P1 | 4 | 3 | 2 | 2 |
| P2 | 4 | 2 | 3 | 2 |
| P3 | 4 | 3 | 3 | 2 |
| P4 | 5 | 2 | 3 | 2 |
| P5 | 4 | 3 | 3 | 2 |
| Mean value Ø | 4.2 | 2.6 | 2.8 | 2.0 |

The results of the evaluation are additionally shown in FIG. 1b.

A clear reduction of the fragrance intensity was observed after 10 min and 25 min, respectively, by incorporating 1,2-alkanediols, 2,3-alkanediols as well as mixtures of the corresponding 1,2-alkanediols and 2,3-alkanediols. In addition, all panelists identified a reduction of a stingy/sharp notes. Especially, Aldehyd C12 NMA has a strong stingy/sharp note, which is usually hard to suppress or to cover. Therefore, the efficient suppression or coverage of the undesired fragrance note of Aldehyd C12 NMA by incorporating 1,2-alkanediols, 2,3-alkanediols as well as mixtures of the corresponding 1,2-alkanediols and 2,3-alkanediols is remarkable. Efficient improvements were found for the use of 1,2-alkanediols or 2,3-alkanediols as such. The particularly remarkable coverage of the afore-mentioned unpleasant fragrance note was especially efficient for compositions comprising 1,2-heptanediols in combination with 2,3-heptanediol and 1,2-octanediol in combination with 2,3-octanediol. No other effect was found out of pure ethanol as fragrance vehicle, therefore the compositions achieve an excellent malodour coverage effect in different media or media compositions. As can be seen from these results the compositions according to the present invention can be efficiently used for malodour reduction and for body odour control. In particular, compositions comprising 1,2-heptanediol, 2,3-heptanediol, 1,2-octanediol and/or 2,3-octanediol provide excellent effects for malodour reduction and for body odour control.

### Example 6: Malodour Coverage Effect on Malodour and Insect Alluring Compounds

Method

Testing Conditions:

The ingredients (1% w/w) in an aqueous/ethanolic solution (7:3) were sprayed on paper stripes and compared with solutions containing alkanediols directly and after 5 min, respectively.

Tested Substances:

TABLE 17
Tested alkanediols.

| Alkanediol | Abbreviation |
|---|---|
| 1,2-Heptanediol | =1,2-C7 |
| 2,3-Heptanediol | =2,3-C7 |
| 1,2-Octanediol | =1,2-C8 |
| 2,3-Octanediol | =2,3-C8 |

TABLE 18
Malodour/insect alluring compound which is a typical
compound found in human sweat.

| Name | Structure |
|---|---|
| 1-Octen-3-ol | 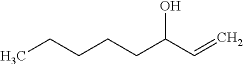 |

TABLE 19
Tested ethanolic/aqueous solution.

| | w/w % | | |
|---|---|---|---|
| | Sample 1 | | |
| Substance | (placebo) | Sample 2 | Sample 3 |
| Ethanol pa | 69.0 | 67.0 | 67.0 |
| Water, dist. | 30.0 | 30.0 | 30.0 |
| 1-Octen-3-ol | 1.0 | 1.0 | 1.0 |
| 1,2-Heptanediol | — | 1.0 | — |
| 2,3-Heptanedol | — | 1.0 | — |
| 1,2-Octanediol | — | — | 1.0 |
| 2,3-Octanedol | — | — | 1.0 |

Preparation of the Fragrance-Solutions:

The 1-octen-3ol was pre-solved in the alkanediol mixtures as specified in Table 19, ethanol added and the solutions vortexed for 15 seconds. Subsequently, water was added and the resulting solutions vortexed again for further 15 seconds.

Results

TABLE 20
Evaluation after 0 minutes (1-octen-3-ol).

| Panelist | Sample 1 | Sample 2 | Sample 3 |
|---|---|---|---|
| P1 | 5 | 1 | 2 |
| P2 | 5 | 4 | 2 |
| P3 | 4 | 3 | 2 |
| P4 | 5 | 3 | 2 |
| P5 | 5 | 4 | 2 |
| P6 | 4 | 3 | 3 |
| Mean value Ø | 4.7 | 3.0 | 2.2 |

TABLE 21
Evaluation after 5 minutes (1-octen-3-ol).

| Panelist | Sample 1 | Sample 2 | Sample 3 |
|---|---|---|---|
| P1 | 4 | 1 | 2 |
| P2 | 4 | 2 | 1 |
| P3 | 3 | 2 | 2 |
| P4 | 4 | 2 | 1 |
| P5 | 4 | 3 | 2 |
| P6 | 4 | 3 | 2 |
| Mean value Ø | 3.8 | 2.2 | 1.7 |

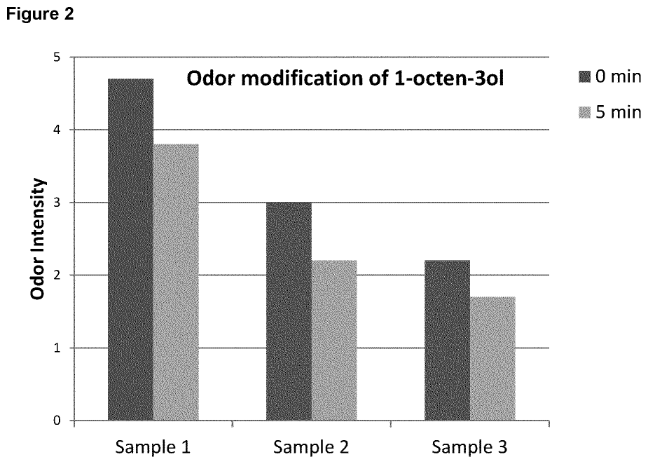

The results of the evaluation are additionally shown in FIG. 2.

A clear reduction of compound intensity was observed directly and after 5 min, respectively, by adding the alkanediol mixtures specified in Table 19. All panelists (P1, P2, P3, P4, P5, P6) identified a significant reduction of the unpleasant earthy/rotten note. Therefore, a strong impact on the spot was observed for the reduction of the malodour of 1-octen-3-ol. The excellent suppression of the malodour of 1-octen-3-ol achieved by the compositions according to the present invention demonstrates that the compositions of the present invention are useful for efficiently masking insect alluring odours and sweat odours. No other effect was found out of pure ethanol as compound vehicle, therefore, the compositions achieve an excellent malodour coverage effect and effect for masking insect alluring odours in different media or media compositions. These results demonstrate that the compositions according to the present invention can be used for pronounced malodour reduction, for body odour control and for masking insect alluring odours.

### Example 7: Boosting/Masking Effect of Alkanediols on Fragrance Ingredients

Testing Conditions:

Paper stripes were dunked in the fragrance solution (1% w/w; aqueous/ethanolic (7:3); see Example 5) and compared with solutions containing alkanediols after 10 and 30 minutes, respectively.

For evaluating the sensorial behaviour of the alkanediols in combination with different odorous compounds, the odour compounds were blended with the alkanediol (mixture) an analysed according to the method described in Example 5 in comparison to the corresponding samples which were prepared without the addition of alkanediol(s) (placebo sample).

The test conditions as well as the FRA-Blend correspond to those used in Example 5 (Tables 10 and 11).

Preparation of the Fragrance-Solutions:

The FRA-Blend specified above (according to Table 11) was pre-solved in the alkanediol (mixture) according to Table 22 and ethanol added. The solution was stirred for 3 min with a magnetic stirrer. Subsequently, water was added, and the resulting solution stirred again for further 3 min.

For evaluating the sensorial behaviour of alkanediols in combination with different odorous compounds, the odour compounds were blended with the alkanediol (mixture) and dissolved in a EtOH/water-solution and assessed by untrained panelists (P1, P2, P3, P4, P5, P6, P7) in comparison to the corresponding samples which were prepared without the addition of alkanediol(s) (placebo sample). The odour intensity was accorded a corresponding value between 1 and 5 (Ranking: 1=weak odour; 5=strong odour/high odour intensity).

TABLE 22
Tested ethanolic/aqueous solutions.

| | w/w % | | | |
|---|---|---|---|---|
| | Sample | | | |
| Substance | A (placebo) | B | C | D |
| Ethanol pa. | 69.0 | 67.0 | 67.0 | 67.0 |
| Water, dist. | 30.0 | 30.0 | 30.0 | 30.0 |
| FRA-Blend | 1.0 | 1.0 | 1.0 | 1.0 |
| 1,2-Heptanediol (1,2-C7) | — | 2.0 | — | 1.0 |
| 2,3-Octanediol (2,3-C8) | — | — | 2.0 | 1.0 |

The odour intensity was accorded a corresponding value between 1 and 5 (Ranking: 1=weak odour; 5=strong odour/high odour intensity).

Results

TABLE 23
Evaluation after 10 and 30 minutes, respectively.

| | Samples after 10 min | | | | Samples after 30 min | | | |
|---|---|---|---|---|---|---|---|---|
| | | | | 1,2-C7 | | | | 1,2-C7 |
| | | | | and | | | | and |
| | | | | 2,3-C8 | | | | 2,3-C8 |
| | Placebo | 1,2-C7 | 2,3-C8 | (1:1) | Placebo | 1,2-C7 | 2,3-C8 | (1:1) |
| | A | B | C | D | A | B | C | D |
| Panelist | | | | | | | | |
| P1 | 5 | 2 | 4 | 3 | 5 | 4 | 3 | 4 |
| P2 | 5 | 2 | 3 | 3 | 5 | 2 | 3 | 2 |
| P3 | 5 | 3 | 4 | 3 | 5 | 3 | 4 | 3 |
| P4 | 5 | 2 | 3 | 2 | 4 | 2 | 3 | 2 |
| P5 | 5 | 3 | 3 | 4 | 4 | 2 | 2 | 3 |
| P6 | 5 | 3 | 3 | 2 | 4 | 2 | 3 | 2 |
| P7 | 5 | 2 | 3 | 5 | 5 | 2 | 3 | 4 |
| Mean | 5.0 | 2.4 | 3.3 | 3.1 | 4.6 | 2.4 | 3.1 | 2.6 |
| value Ø | | | | | | | | |

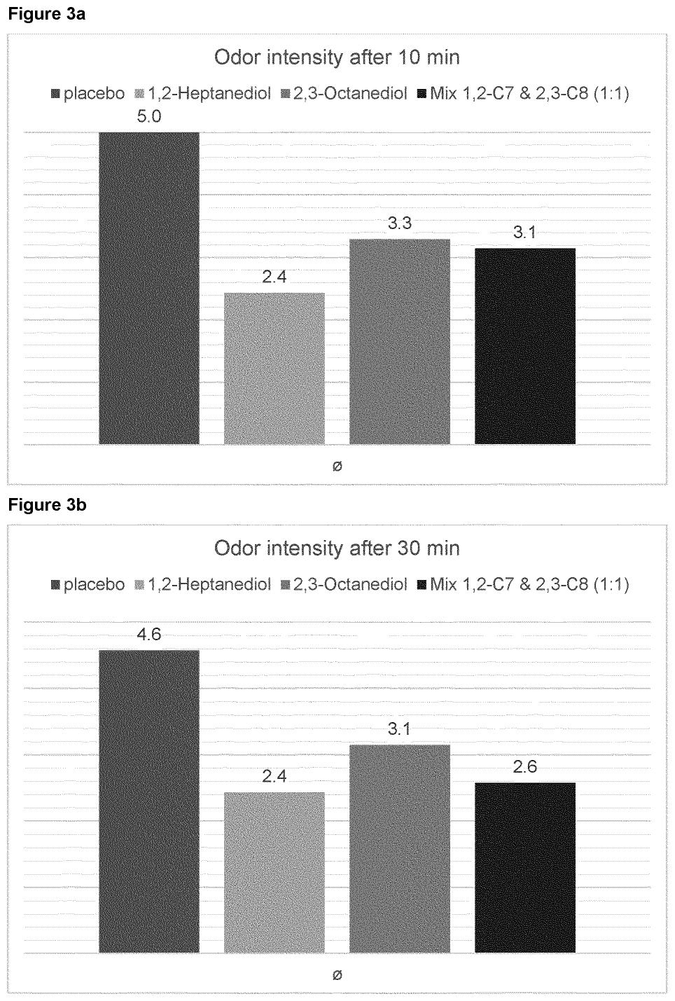

The results of the evaluation are additionally shown in FIGS. 3a and 3b, respectively.

TABLE 24
Evaluation after 10 and 30 minutes, respectively—
sorted results based on masking effect.
10 min

| | Placebo | | 2,3-C8 | | Blend of 1,2-C7 and | | 1,2-C7 |
|---|---|---|---|---|---|---|---|
| Sample | (A) | > | (C) | > | 2,3-C8 (1:1) (D) | > | (B) |
| Fragrance | 5.0 | | 3.3 | | 3.1 | | 2.4 |
| intensity | | | | | | | |

30 min

| | Placebo | | 2,3-C8 | | Blend of 1,2-C7 and | | 1,2-C7 |
|---|---|---|---|---|---|---|---|
| Sample | (A) | > | (C) | > | 2,3-C8 (1:1) (D) | > | (B) |
| Fragrance | 4.6 | | 3.1 | | 2.6 | | 2.4 |
| intensity | | | | | | | |

Based on the above results it can be concluded that both the alkanediols as such as well as mixtures of the 1,2-alkanediols with 2,3-alkanediols efficiently mask the fragrance blend. Thereby, 1,2-heptanediol masks the fragrance the most, followed by the mixture of the two diols differing in their chain lengths (hetero combination). 2,3-Octandiol alone shows the lowest masking effect in the present trial.

### D) Formulation Examples

The formulation examples below relate to the following applications:
Deodorant Spray
Deodorant Spray with AcH
Deodorant Roll-on
Cool Relief Deo Plus
Sport Fit Deo
Antiperspirant/deodorant roll-on
Deodorant formulation in the form of a roll-on-gel
Clear deo anti-perspirant roll-on
Deodorant Aerosol
Deodorant Stick
Antiperspirant Stick
Intimate Sensi-Gel
Mild feminine wash
Intimate Rescue Gel
After-Sun Refreshing Gel
Cool & Hot Firming GEL with Thermolat & Frescolat
Men After-Shave Fluid
After-Shave
Frosty Facial Foam
Fresh Hair Antidandruff Shampoo
Anti-Oil Shampoo
Detox Refreshing Spray for Hair
Scalp soothing hair conditioner with UV-B/UV-A protection, rinse off
Hair Soap SHAMPOO Bar
Hair Refreshener Mist
Refreshing conditioning Foam
Virgin Mojito Pre-Shampoo
Anti-itch hair conditioner, leave on
Low-Cost Refreshing Body Lotion
Intensive Care, Detox & Repair Hand Cream 2 in1
Hygiene & Care Hand Cream
Refreshing and Soothig After Sun Cream
Daily Well Aging with SPF 30, UVA/UVB balanced w/o Octocrylene
Mosquito & tick repellent Spray
Insect Repellent Spray, aqueous/ethanolic
Repellent solution for wipes
Anti-Sweat Sunscreen Lotion
HH-Cream Haute Homme with exp. PF15 UVA/UVB
BB Cream Dark Tone SPF-20 (Expected)
Cool Sebum Control Cleanser
Refreshing Lip Gloss
Refresh your Breath Mouthspray
Syndet antimicrobial soap bar
Syndet soap bar
Ice Crystal Soap
Antimicrobial toilet soap bar
Air freshener (NF spray water based)

The perfume oils PO1, PO2, PO3, PO4, or PO5 from the below examples were worked separately in each case into the formulations presented.

TABLE 25
Composition of perfume oil 1 (PO1, amounts in ‰ b.w.)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| BENZYLACETATE | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2,4 10% IPM | 2 |
| FLOROSA | 5 |
| FRAMBINON ® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

TABLE 26
Composition of perfume oil 2 (PO2, amounts in ‰ b.w.)

| Ingredients | Amount |
|---|---|
| Acetophenone, 10% in DPG | 10 |
| n-Undecanal | 5 |
| Aldehyde C14, so-called (peach aldehyde) | 15 |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyloxide | 5 |
| Eucalyptol | 10 |
| Geraniol | 40 |
| Nerol | 20 |
| Geranium oil | 15 |
| Hexenol cis-3, 10% in DPG | 5 |
| Hexenyl salicylate cis-3 | 20 |
| Indole, 10% in DPG | 10 |
| Alpha-ionone | 15 |
| Beta-ionone | 5 |
| Lilial ® (2-methyl-3-(4-tert-butyl-phenyl)propanal) | 60 |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |
| Total: | 1000 |

TABLE 27
Composition of perfume oil 3 (PO3, amounts in ‰ b.w.)

| Ingredients | Amount |
|---|---|
| Benzyl acetate | 60 |
| Citronellyl acetate | 60 |
| Cyclamenaldehyde (2-methyl-3-(4-isopropyl- | 20 |
| phenyl)propanal | |
| Dipropylene glycol (DPG) | 60 |
| Ethyllinalool | 40 |
| Florol (2-isobutyl-4-methyltetrahydro-2H-pyran-4-ol) | 30 |
| Globanone ® [(E/Z)-8-cyclohexadecen-1-one] | 180 |
| Hedione ® (methyldihydrojasmonate) | 140 |
| Hexenyl salicylate, cis-3 | 10 |
| Vertocitral (2,4-dimethyl-3- | 5 |
| cyclohexenecarboxaldehyde) | |
| Hydratropaldehyde, 10% in DPG | 5 |
| Isodamascone (1-(2,4,4-trimethyl-2-cyclohexen-1- | 5 |
| yl)-2-buten-1-one, 10% in DPG | |
| Isomuscone (cyclohexadecanone) | 40 |
| Jacinthaflor (2-methyl-4-phenyl-1,3-dioxolane) | 10 |
| Cis-jasmone, 10% in DPG | 20 |
| Linalool | 50 |
| Linalyl acetate | 30 |
| Methyl benzoate, 10% in DPG | 25 |
| para-Methyl cresol, 10% in DPG | 10 |
| Nerol | 20 |
| Phenylpropylaldehyde | 5 |
| 2-Phenylethyl alcohol | 82 |
| Tetrahydrogeraniol | 13 |
| 2,2-Dimethyl-3-cyclohexyl-1-propanol | 80 |
| Total: | 1000 |

TABLE 28
Composition of perfume oil 4 (PO4, amounts in ‰ b.w.)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL, bergapten-free | 60 |
| CALONE ® 1951 10% in DPG | 15 |
| COUMARIN | 5 |
| CYCLOGALBANATER 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDER (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL | 1000 |

TABLE 29
Composition of perfume oil 5 (PO5, amounts in ‰ b.w.)

| Ingredients | Amount |
|---|---|
| AMAROCITE ® | 10 |
| AMBROCENIDE ® 10% in DPG | 5 |
| AMBROXIDE | 15 |
| AURELIONE ® (7/8-Cyclohexadecenone) (MACRO) | 70 |
| BERGAMOT OIL, bergapten-free | 90 |
| CALONE ® 1951 10% in DPG | 20 |
| CARAWAY OIL | 10 |
| CITRAL | 20 |
| COUMARIN | 10 |
| ALPHA-DAMASCONE 1% in DPG | 15 |
| DIHYDROMYRCENOL | 70 |
| ESTRAGON OIL | 10 |
| ETHYL LINALOOL | 100 |
| ETHYL LINALYLACETATE | 90 |
| EUGENOL | 10 |
| EVERNYL ® | 5 |
| FRUCTATE ® | 5 |
| GERANIUM OIL | 5 |
| HEDIONE ® HC/30 | 100 |
| HELIONAL ® | 10 |
| INDOLE 10% in DPG | 5 |
| ISO E SUPER ® | 100 |
| KEPHALIS ® | 5 |
| LAVENDER OIL | 40 |
| CITRUS OIL | 80 |
| LILIAL ® | 30 |
| MANDARIN OIL | 20 |
| MUSCENONE (MACRO) | 5 |
| SANDRANOL ® | 10 |
| VANILLIN 10% in DPG | 5 |
| DIPROPYLENE GLYCOL | 30 |
| TOTAL | 1000 |

The perfume oils PO1, PO2, PO3, PO4, or PO5 from the above examples were worked separately in each case into the formulations presented below.

Cosmetic formulations (compositions)—amounts are indicated as % by weight for all formulations:

TABLE 30
Deodorant Spray

| Ingredients | EU INCI | Amount |
|---|---|---|
| Aqua/Water | Aqua | ad 100 |
| Volarest ™ FL | Acrylates/Beheneth-25 | 1.2 |
| | Methacrylate copolymer | |
| Dracorin ® GOC | Glyceryl oleate citrate; | 2.0 |
| | Caprylic/Capric triglyceride | |
| SymDeo ® Plus | Lauryl alcohol, Phenoxyethanol; 2- | 0.5 |
| | Benzylheptanol; Decylene Glycol | |
| SymOcide ® PS | Phenoxyethanol; Decylene Glycol; | 0.8 |
| | 1,2-Exanediol | |
| Dow Corning ® 245 Fluid | Cyclomethicone | 5.0 |
| Isopropyl Myristate | Isopropyl myristate | 2.0 |
| Farnesol | Farnesol | 0.25 |
| Ethylhexylglycerin | Ethylhexylglycerin | 0.2 |
| SymDeo ® B125 | 2-Methyl 5-Cycloheylpentanol | 0.15 |
| Tego ® Cosmo P 813MB | Polyglyceryl-3 Caprylate | 0.25 |
| Perfume oil PO1, PO2, | Parfum | 0.8 |
| PO3, PO4, or PO5 | | |
| Frescolat ® ML nat. | Menthyl Lactate | 0.2 |
| Menthol | Menthol | 0.1 |
| Sodium Hydroxide, | Aqua; Sodium hydroxide | 0.6 |
| 10% aqueous | | |
| 1,2-Heptanediol | 1,2-Heptanediol | 0.5 |
| 2,3-Heptanediol | 2,3-Heptanediol | 0.02 |
| 1,2-Nonanediol | 1,2-Honanediol | 0.2 |
| 2,3-Nonandiol | 2,3-Nonanediol | 0.01 |

TABLE 31
Deodorant Spray with AcH

| Ingredients | EU INCI | Amount |
|---|---|---|
| Triethylcitrate | Triethylcitrate | 3.0 |
| Menthol | Menthol | 0.1 |
| Solubilizer ® | PEG-40 Hydrogenated | 1.0 |
| | Castor Oil, Trideceth-9, | |
| | Propylene Glycol, Aqua | |
| Ethanol 96% | Ethanol | 30.0 |
| 1,2-Nonanediol | 1,2-Nonanediol | 0.5 |
| 2,3-Nonanediol | 2,3-Nonanediol | 0.05 |
| 2,3-Octanediol | 2,3-Octanediol | 0.1 |
| Ethylhexylglycerin | Ethylhexylglycerin | 0.3 |
| SymDeo ® B125 | 2-Methyl 5-Cycloheylpentanol | 0.25 |
| SymClariol ® | Decylene Glycol | 0.25 |
| Frescolat ® ML | Menthyl Lactate | 0.6 |
| Tego ® Cosmo P 813MB | Polyglyceryl-3 Caprylate | 0.5 |
| Locron ® L (50%) | Aqua, Aluminum Chlorohydrate | 25.0 |
| Perfume oil PO1, PO2, | Parfum | 0.5 |
| PO3, PO4, or PO5 | | |
| Water, demin. | Aqua | ad 100 |

TABLE 32
Deodorant Roll-on

| Ingredients | EU INCI | Amount |
|---|---|---|
| Aqua/Water | Aqua | ad 100 |
| Hydrolite ® CG | Caprylyl Glycol | 0.3 |
| 1,2-Heptanediol | 1,2-Heptanediol | 1.0 |
| 2,3-Heptanediol | 2,3-Heptanediol | 0.05 |
| 1,2-Nonanediol | 1,2-Nonanediol | 0.1 |
| 2,3-Nonandiol | 2,3-Nonanediol | 0.05 |
| Menthol | Menthol | 0.1 |
| Frescolat ® ML | Menthyl Lactate | 1.0 |
| SymSave ® H | Hydroxyacetophenone | 0.5 |
| EDTA NA2 | Disodium EDTA | 0.1 |
| Dracorin ® GOC | Glyceryl oleate citrate; | 4.0 |
| | Caprylic/Capric triglyceride | |
| Dragoxat ® 89 | Ethylhexyl isononanoate | 5.0 |
| SymMollient ® S | Cetearyl Nonanoate | 1.0 |
| Carbopol ® Ultrez | Acrylates/C10-30 Alkyl | 0.2 |
| 20 Polymer | acrylate crosspolymer | |
| Pemulen ™ TR-2 | Acrylates/C10-30 Alkyl | 0.2 |
| Polymeric Emulsifier | acrylate crosspolymer | |
| Farnesol | Farnesol | 0.3 |
| Sym Guard ® CD | Phenylpropanol, o-Cymen- | 0.3 |
| | 5-ol, Decylene Glycol | |
| Perfume oil PO1, PO2, | Parfum | 0.5 |
| PO3, PO4, or PO5 | | |
| Sodium Hydroxide, | Aqua; Sodium hydroxide | 0.2 |
| 50% solution | | |

TABLE 33
Cool Relief Deo Plus

| Ingredients | INCI | Amount |
|---|---|---|
| Aqua/Water | Aqua | ad 100 |
| Veegum ® HS Granules | Magnesium Aluminium Silicate | 1.0 |
| Glycerin | Glycerin | 5.0 |
| EDTA NA2 | Disodium EDTA | 0.1 |
| Dracorin ® 100 SEP | Glyceryl Stearate, PEG-100 | 6.0 |
| | Stearate | |
| Lanette 16 | Cetyl alcohol | 2.0 |
| Eumulgin ® B2 | Ceteareth-20 | 6.0 |
| Eutanol ® G | Octyldodecanol | 8.0 |
| Dow Cornng FZ- | Caprylyl Methicone | 4.0 |
| 3196 Volatile | | |
| Tween 20 | Polysorbate20 | 0.3 |
| SymDeo ® B125 | 2-Methyl 5-Cyclohexylpentanol | 0.3 |
| Dragosantol ® 100 | Bisabolol | 0.05 |
| Aqua/Water | Aqua | 15.0 |
| Locron ® P | Aluminium Chlorohydrate | 15.0 |
| 1,2-Nonanediol/2,3- | 1,2-Nonanediol/2,3-Monanediol | 1.0 |
| Nonanediol (95:5 w/w %) | | |
| SymOcide ® PS | Phenoxyethanol, Decylene | 1.0 |
| | Glycol, 1,2-Hexanediol | |
| Frescolat ® ML | Menthyl Lactate | 0.5 |
| Vitacel ® CS020 | Cellulose | 1.0 |
| Extrapone ® Rose | Aqua, Glycerin, Xanthan Gum, | 1.0 |
| Quartz GW | Quartz Powder | |
| Perfume oil PO1, PO2, | Parfum | 1.0 |
| PO3, PO4, or PO5 | | |
| Sodium hydroxide | Aqua, Sodium hydroxide | 0.5 |
| 10% solution | | |

TABLE 34
Sport Fit Deo

| Ingredients | INCI | Amount |
|---|---|---|
| SymLite ® G8 | Glyceryl caprylate | 0.15 |
| Menthol | Menthol | 0.05 |
| Ethanol | Alcohol denat | ad 100 |
| SymClariol ® | Decylene Glycol | 0.25 |
| 1,2-Heptanediol, 2,3- | 1,2-Heptanediol, | 0.5 |
| Heptanediol (95:5 w/w %) | 2,3-Heptanediol | |
| SymDeo ® B125 | 2-Methyl 5-Cyclohexylpentanol | 0.25 |
| Frescolat ® ML | Menthyl Lactate | 0.5 |
| Extrapone ® Ginseng | Propylene Glycol, Aqua, Panax | 0.1 |
| | ginseng root extract, Lactic acid | |
| Extrapone ® Peppermint | Aqua, Propylene Glycol, Mentha | 0.1 |
| | Piperita Leaf extract | |
| Ethylhexyglycerin | Ethylhexylglycerin | 0.2 |
| Perfume oil PO1, PO2, | Parfum | 1.0 |
| PO3, PO4, or PO5 | | |

TABLE 35
Antiperspirant/deodorant roll-on

| Ingredients | INCI | Amount |
|---|---|---|
| SymLite ® G8 | Glyceryl caprylate | 0.15 |
| Dragosantol ® 100 | Bisabolol | 0.1 |
| 1,2-Heptanediol | 1,2-Heptanediol | 0.1 |
| 2,3-Heptanediol | 2,3-Heptanediol | 0.005 |
| 1,2-Nonanediol | 1,2-Nonanediol | 0.15 |
| 2,3-Nonandiol | 2,3-Nonanediol | 0.01 |
| Ethanol 96% | Ethanol | 30.0 |
| Farnesol | Farnesol | 0.5 |
| Perfume oil PO1, PO2, | Parfum | 1.5 |
| PO3, PO4, or PO5 | | |
| Frescolat ® ML cryst. | Menthyl Lactate | 0.2 |
| Irgasan ® DP 300 | Triclosan | 0.3 |
| Natrosol ® 250 HHR | Hydroxyethyl-cellulose | 0.3 |
| Solubilizer 611674 | PEG-40 Hydrogenated Castor | 2.0 |
| | Oil, Trideceth-9, Water (Aqua) | |
| SymDeo ® B125 | 2-Methyl 5-Cyclohexylpentanol | 0.5 |
| Water (demineralized) | Water (Aqua) | ad 100 |
| Zirkonal L 450 | Aluminium Zirconium | 37.0 |
| | Pentachloro-hydrate (40% | |
| | aqueous solution) | |

TABLE 36
Deodorant formulation in the form of a roll-on gel

| Ingredients | INCI | Amount |
|---|---|---|
| 1,3-Butylene glycol | 1,3-Butylene glycol | 2.0 |
| Cremophor ® CO 40 | PEG-40-hydrogenated | 2.0 |
| Surfactant | castor oil | |
| Hydroxyethylcellulose | Hydroxyethylcellulose | 0.5 |
| Perfume oil PO1, PO2, | Parfum | 0.3 |
| PO3, PO4, or PO5 | | |
| 1,3-Propanediol | 1,3-Propanediol | 0.5 |
| Sym Guard ® CD | 3-Phenylpropanol, | 0.4 |
| | o-cymen-3-ol, | |
| | Decylene glycol | |
| Ethylhexylglycerin | Ethylhexylglycerin | 0.1 |
| 1,2-Heptanediol | 1,2-Heptanediol | 0.5 |
| 1,2-Nonanediol, 2,3- | 1,2-Nonanediol, | 0.5 |
| Nonanediol (95:5 w/w %) | 2,3-Nonanediol | |
| SymLite ® G8 | Glyceryl Caprylate | 0.15 |
| Frescolat ® ML cryst. | Menthyl Lactate | 0.4 |
| Water | Aqua | ad 100 |

TABLE 37
Clear deo anti-perspirant roll-on

| Ingredients | INCI | Amount |
|---|---|---|
| Methocel ™ E4M Premium | Hydroxypropyl | 0.5 |
| | Methylcellulose | |
| Water | Water (Aqua) | ad 100 |
| Neo-PCL Water Soluble N | Trideceth-9, PEG-5 | 1.0 |
| | Ethylhexanoate, | |
| | Water (Aqua) | |
| Solubilizer | PEG-40 Hydrogenated | 3.0 |
| | Castor Oil, Trideceth-9, | |
| | Propylene Glycol, | |
| | Water (Aqua) | |
| Deolite | Dimethyl Phenylpropanol, | 0.5 |
| | Pentylene Glycol | |
| Locron ® LW | Aluminium Chlorohydrate | 25.0 |
| Aloe Vera Gel Concentrate 10/1 | Aloe Barbadensis Leaf Juice | 1.0 |
| 1,2-Propylene Glycol 99 P GC | Propylene Glycol | 4.0 |
| Ethanol 96% | Alcohol Denat. | 30.0 |
| Frescolat ® ML | Menthyl Lactate | 1.5 |
| Perfume oil PO1, PO2, PO3, | Parfum | 1.0 |
| PO4, or PO5 | | |
| 1,2-Heptanediol, 2,3-Heptanediol | 1,2-Heptanediol, | 1.0 |
| (w/w % 95:5) | 2,3-Heptanediol | |

TABLE 38
Deodorant Aerosol

| Ingredients | EU INCI | Amount |
|---|---|---|
| Belsil ® DM 0.65 | Disiloxane | ad 100 |
| Isoadipate | Diisopropyl adipate | 5.0 |
| Tegosoft ® TN | C12-15 Alkyl benzoate | 10.0 |
| Vitamin E Acetate | Tocopheryl acetate | 0.5 |
| Farnesol | Farnesol | 0.3 |
| Ethylhexylglycerin | Ethylhexylglycerin | 0.2 |
| 2,3-Heptanediol | 2,3-Heptanediol | 0.1 |
| 1,2-Nonanediol | 1,2-Nonanediol | 0.2 |
| SymDeo ® MPP | Dimethyl Phenylbutanol | 0.2 |
| Frescolat ® ML nat. | Menthyl Lactate | 0.25 |
| Perfume oil PO1, PO2, PO3, PO4, or | Parfum | 0.6 |
| PO5 | | |

TABLE 39
Deodorant stick

| Ingredients | INCI | Amount |
|---|---|---|
| Sodium stearate | Sodium stearate | 8.0 |
| PPG-3 Myristyl ether | PPG-3 Myristyl ether | 70.0 |
| 1,2-Propylene glycol | 1,2-Propylene glycol | 10.0 |
| 1,1-Dimethyl-3- | 1,1-Dimethyl-3- | 0.2 |
| phenylpropanol | phenylpropanol | |
| 2-Butyloctanoic acid | 2-Butyloctanoic acid | 0.2 |
| Perfume oil PO1, PO2, PO3, | Parfum | 0.6 |
| PO4, or PO5 | | |
| Water | Aqua | ad 100 |
| Menthol | Menthol | 0.01 |
| SymDeo ® Plus | Lauryl Alcohol, | 0.5 |
| | Phenoxyethanol, | |
| | 2-Benzylheptanol, | |
| | Decylene Glycol | |
| 3-((−)-Menthoxy)propane- | 3-((−)-Menthoxy)propane- | 0.1 |
| 1,2-diol | 1,2-diol | |
| N-Ethyl-p-menthane-3- | Ethyl Menthane Carboxamide | 0.3 |
| carboxamide | | |
| (Cooling Agent WS-3) | | |
| SymLite ® G8 | Glyceryl Caprylate | 0.15 |
| 1,2-Heptanediol | 1,2-Heptanediol | 0.50 |

TABLE 40
Antiperspirant stick

| Ingredients | INCI | Amount |
|---|---|---|
| PCL-Liquid ® 100 | Cetearyl ethylhexanonate | ad 100 |
| Silicone Fluid 345 | Cyclomethicone | 10.0 |
| CRODACOL ™ C90 | Cetyl Alcohol | 8.0 |
| SYNCROWAX ™ HGLC | C18-36 Triglyceride | 8.0 |
| CRODAMOL ™ PTC | Pentaerythritol | 5.0 |
| | Tetracaprylate/Caprate | |
| SYNCROWAX ™ HRC | Tribehenin | 4.0 |
| Brij ™ 05 | Oleth-5 | 1.0 |
| Titanium Dioxide | Titanium Dioxide | 1.0 |
| Menthol | Menthol | 0.05 |
| Frescolat ® ML Nat | Menthyl Lactate | 0.75 |
| Rezal 36GP | Aluminium | 20.0 |
| | Tetrachlorohydrex GLY | |
| Dry-Flo ® C | Aluminium Starch Octenyl | 22.5 |
| | Succinate | |
| N-Ethyl-p-menthane-3- | Ethyl Menthane | 0.5 |
| carboxamide | Carboxamide | |
| (Cooling Agent WS-3) | | |
| Frescolat ® MGA | Menthone Glycerin Acetal | 0.6 |
| Preservative | Phenoxyethanol | 0.8 |
| Perfume oil PO1, PO2, PO3, | Parfum | 0.6 |
| PO4, or PO5 | | |
| 1,2-Heptanediol, 2,3-Heptanediol | 1,2-Heptanediol, | 0.5 |
| (95:5 w/w %) | 2,3-Heptanediol | |
| 2,3-Nonanediol | 2,3-Nonanediol | 0.05 |

TABLE 41
Intimate Sensi-Gel

| Ingredients | EU INCI | Amount |
|---|---|---|
| Aqua/Water | Aqua | ad 100 |
| Sodium Benzoate | Sodium benzoate | 0.3 |
| Keltrol ® CG-T | Xanthan Gum | 0.2 |
| Cyamopsis Tetragonolobus | Cyamopsis Tetragonolobus | 0.1 |
| Gum | Gum | |
| Glycerin | Glycerin | 4.0 |
| Tego ® Betain F50 | Cocamidopropyl betaine | 14.0 |
| Solubilizer ® | PEG-40 Hydrogenated castor oil, | 3.0 |
| | Trideceth-9, Propylene Glycol, | |
| | Aqua | |
| 1,2-Heptanediol | 1,2-Heptanediol | 0.5 |
| Hydrolite ® 6 | 1,2-Hexanediol | 1.0 |
| SymGuard ® CD | Phenylpropanol, o-cymen-5-ol, | 0.3 |
| | Decylene Glycol | |
| SymRelief ® 100 | Bisabolol, Zingiber officinale | 0.1 |
| | root extract | |
| Perfume oil PO1, PO2, | Parfum | 0.2 |
| PO3, PO4, or PO5 | | |
| Frescolat ® X-cool | Menthyl ethylamido oxalate | 0.4 |
| Frescolat ® Plus | Menthol, Menthyl Lactate | 0.3 |
| Plantacare 2000 UP | Decyl Glucoside | 2.0 |
| Lactic acid 90% Nat. | Lactic Acid, Aqua | 0.3 |

TABLE 42
Mild feminine wash

| Ingredients | EU INCI | Amount |
|---|---|---|
| Aqua/Water | Aqua | ad 100 |
| SymSave ® H | Hydroxyacetophenone | 0.5 |
| Keltrol CG-T | Xanthan Gum | 0.5 |
| Glycerin | Glycerin | 2.0 |
| Propylene Glycol | Propylene Glycol | 3.0 |
| 1,2-Heptanediol | 1,2-Heptanediol | 0.5 |
| 2,3-Heptanediol | 2,3-Heptanediol | 0.03 |
| Tego Betain F50 | Cocamidopropyl betaine | 14.0 |
| SymGuard ® CD | Phenylpropanol, o-cymen-5-ol, | 0.3 |
| | Decylene Glycol | |
| Frescolat ® ML nat. | Menthyl Lactate | 0.2 |
| Plantacare ® 2000 UP | Decyl Glucoside | 2.0 |
| Extrapone ® Witch | Glycerin, Aqua, Hamamelis | 1.0 |
| Hazel GW | Virginiana Bark/-Leaf/ | |
| | Twig Extract | |
| Solubilizer | PEG-40 Hydrogenated castor oil, | 1.5 |
| | Trideceth-9, Propylene Glycol, | |
| | Aqua | |
| Perfume oil PO1, PO2, | Parfum | 0.2 |
| PO3, PO4, or PO5 | | |
| SymCalmin ® | Pentylene Glycol, Butylene Blycol, | 1.0 |
| | Hydroxyphenyl propamidobenzoic | |
| | acid | |
| Lactic acid 90% Nat. | Lactic Acid, Aqua | 0.32 |

TABLE 43
Intimate Rescue Gel

| Ingredients | EU INCI | Amount |
|---|---|---|
| Aqua/Water | Aqua | ad 100 |
| SymSave ® H | Hydroxyacetophenone | 0.3 |
| Keltrol CG-T | Xanthan Gum | 0.3 |
| Genuvisco ® CG-131 | Carrageenan | 0.2 |
| Glycerin | Glycerin | 4.0 |
| Tego ® Betain F50 | Cocamidopropyl betaine | 14.0 |
| Sodium Hydroxide 10% | Aqua, Sodium hydroxide | 1.0 |
| solution | | |
| Solubilizer | PEG-40 Hydrogenated | 3.0 |
| | castor oil, Trideceth-9, | |
| | Propylene Glycol, | |
| | Aqua | |
| Hydrolite ® 6 | 1,2-Hexanediol | 1.0 |
| 1,2-Heptanediol, | 1,2-Heptanediol, | 0.475 |
| 2,3-Heptanediol | 2,3-Heptanediol | |
| (95:5 w/w %) | | |
| 2,3-Nonanediol | 2,3-Nonanediol | 0.025 |
| SymGuard ® CD | Phenylpropanol, o-cymen-5-ol, | 0.3 |
| | Decylene Glycol | |
| SymCalmin ® | Pentylene Glycol, Butylene Blycol, | 1.0 |
| | Hydroxyphenyl propamidobenzoic | |
| | acid | |
| Perfume oil PO1, PO2, | Parfum | 0.2 |
| PO3, PO4, or PO5 | | |
| Frescolat ® MGA Plus | Menthone Glycerin Acetal, | 0.5 |
| | Menthol | |
| Plantacare 2000 UP | Decyl Glucoside | 2.0 |

TABLE 44
After-Sun Refreshing Gel

| Ingredients | EU INCI | Amount |
|---|---|---|
| Aqua/Water | Aqua | ad 100 |
| Hydrolite ® 5 green | Pentylene Glycol | 2.5 |
| SymSave ® H | Hydroxyacetophenone | 0.5 |
| Panthenol 75W | Panthenol | 0.5 |
| 1,3-Butylene Glycol | Butylene Glycol | 3.0 |
| Glycerin 99.5% | Glycerin | 2.0 |
| DragoCalm ® | Aqua, Glycerin, Avena sativa | 1.5 |
| | kernel extract | |
| SymGlucan ® | Aqua, Glycerin, 1,2-Hexanediol, | 5.0 |
| | Caprylyl Glycol, Beta-Glucan | |
| Dragosine ® | Carnosine | 0.1 |
| Nativacare ™ 5600 | Zea (Mays) Corn Starch | 2.0 |
| Guar Gum | Guar Gum | 0.1 |
| Rheocare ® XGN | Xanthan Gum | 0.1 |
| Solubilizer ® | PEG-40 Hydrogenated castor oil, | 1.5 |
| | Trideceth-9, Propylene Glycol, | |
| | Aqua | |
| SymMollient ® W/S | Trideceth-9, PEG-5 | 1.0 |
| | Isononanoate, Aqua | |
| SymBright ® 2036 | Sclareolide | 0.1 |
| SymSitive ® 1609 | Pentylene Glycol, 4-t- | 1.0 |
| | Butylcyclohexanol | |
| SymUrban ® | Benzylidene | 0.15 |
| | Dimethoxydimethylidanone | |
| 1,2-Heptanediol, | 1,2-Heptanediol, | 1.0 |
| 2,3-Heptanediol | 2,3-Heptanediol | |
| (w/w % 95:5) | | |
| Frescolat ® ML Cryst. | Menthyl Lactate | 0.3 |
| N-Ethyl-p-menthane-3- | Ethyl Menthane Carboxamide | 0.2 |
| carboxamide | | |
| (cooling agent WS-3) | | |
| Perfume oil PO1, PO2, | Parfum | 0.05 |
| PO3, PO4, or PO5 | | |
| Sodium Hydroxide 10% | Aqua, Sodium hydroxide | 0.8 |
| solution | | |
| Extrapone ® Cucumber | Aqua, Propylene Glycol, cucumis | 1.0 |
| CL | sativus (Cucumber) Juice | |
| SymFinity ® 1298 | Echinacea Purpurea Extract | 0.06 |

TABLE 45
Cold & Hot Firming GEL with Thermolat & Frescolat

| Ingredients | EU INCI | Amount |
|---|---|---|
| Aqua/Water | Aqua | ad 100 |
| Actipone ® Orange | Aqua, Propylene Glycol, Citrus | 0.5 |
| Flower | aurantium amara flower extract | |
| Cosmedia ® SP | Sodium Polyacrylate | 1.2 |
| PCL-Solid | Stearyl Heptanoate, Stearyl | 3.5 |
| | Caprylate | |
| 1,2-Heptanediol, | 1,2-Heptanediol, | 1.0 |
| 2,3-Heptanediol | 2,3-Heptanediol | |
| (w/w % 95:5) | | |
| 1,2-Nonanediol | 1,2-Nonanediol | 0.2 |
| Isodragol | Triisononanoin | 5.0 |
| Caprylic/Capric | Caprylic/Capric Triglyceride | 1.5 |
| Triglyceride | | |
| Jojoba Oil | Simmondsia chinensis seed oil | 1.5 |
| SymFit ® 1617 | Trimethylcyclohexy | 0.2 |
| | Butylcarbamate | |
| Tocopheryl Acetate | Tocopheryl Acetatet | 0.3 |
| Thermolat ® | Vanillyl Butyl Ether, 1,2- | 1.0 |
| | Hexanediol, Caprylyl Glycol | |
| Frescolat ® X-Cool | Menthyl Ethylamido oxalate | 2.0 |
| 3-((−)-Menthoxy)propane- | 3-((−)-Menthoxy)propane-1,2-diol | 0.1 |
| 1,2-diol | | |
| SymOcide ® PS | Phenoxyethanol, Decylene | 1.0 |
| | Glycol, 1,2-Hexanediol | |
| Perfume oil PO1, PO2, | Parfum | 0.5 |
| PO3, PO4, or PO5 | | |
| Citric Acid 30% sol. | Aqua, Citric acid | 0.15 |

TABLE 46
Men After-Shave Fluid

| Ingredients | EU INCI | Amount |
|---|---|---|
| Water demin. | Aqua | ad 100 |
| Glycerin | Glycerin | 3.00 |
| 1,2-Heptanediol, | 1,2-Heptanediol, | 0.5 |
| 2,3-Heptanediol | 2,3-Heptanediol | |
| (w/w % 95:5) | | |
| Dermosoft ® 1388 | Water, Glycerin, Sodium | 3.00 |
| | Levulinate, | |
| | Sodium Anisate | |
| Tego ® Care CG90 | Cetearyl Glucoside | 1.00 |
| Covathick 2009 | Microcrystalline Cellulose, | 0.50 |
| | Cellulose Gum | |
| Cutina ® GMS V | Glyceryl Stearate | 1.00 |
| Coconut Oil refined, organic | Cocos Nucifera (Coconut) Oil | 10.00 |
| Dermofeel ® BGC | Butylene Glycol | 5.00 |
| | Dicaprylate/Dicarate | |
| Keltrol ® CG-SFT | Xanthan Gum | 0.30 |
| Genuvisco ® CG-131 | Carrageenan | 0.30 |
| Citric acid solution 10% | Water, citric acid | 1.70 |
| Ethanol | Alcohol | 5.00 |
| Perfume oil PO1, PO2, | Parfum | 0.50 |
| PO3, PO4, or PO5 | | |
| Dragosantol ® 100 | Bisabolol | 0.2 |
| Frescolat ® X-cool | Menthyl Ethylamido oxalate | 1.0 |
| SymCalmin ® Green | Butylene Glycol, Pentylene | 2.0 |
| | Glycol, Hydroxyphenyl | |
| | Propamidobenzoic acid | |

TABLE 47
After-Shave

| Ingredients | EU INCI | Amount |
|---|---|---|
| Dracorin ® GOC | Glycery Oleate Citrate, | 1.0 |
| | Caprylic/Capric Triglyceride | |
| Dragoxat ® 89 | Ethylhexyl Isononanoate | 5.0 |
| SymMollient ® S | Cetearyl Nonanoate | 1.0 |
| Tocopheryl Acetate | Tocopheryl Acette | 0.25 |
| Aqua/Water | Aqua | ad 100 |
| Pemulen ™ TR-2 | Acrylates/C10-30 Alkyl Acrylate | 0.3 |
| | Crosspolymer | |
| Aloe Vera Gel concentrate | Aloe Barbadensis leaf juice | 0.5 |
| 10/1 | | |
| SymCare ® W2 | PEG-40 Hydrogenated castor oil, | 1.0 |
| | Trideceth-9, Pentylene Glycol, 4-t- | |
| | Butylcyclohexanol, Propylene | |
| | Glycol, Aqua, Hydroxyphenyl | |
| | propamidobenzoic acid | |
| Glycerin | Glycerin | 3.0 |
| SymDiol ® 68 | 1,2-Hexanediol, Caprylyl Glycol | 0.5 |
| SynSave ® H | Hydroxyacetophenone | 0.5 |
| SymGlucan ® | Aqua, Glycerin, 1,2-Hexanediol, | 0.2 |
| | Caprylyl Glycol, Beta-Glucan | |
| Extrapone ® Glacier Water | Glycerin, Aqua | 1.0 |
| GW Allantoin | Allantoin | 0.15 |
| Sodium Hydroxide 10% | Aqua, Sodium Hydroxide | 0.54 |
| solution | | |
| Perfume oil PO1, PO2, | Parfum | 1.0 |
| PO3, PO4, or PO5 | | |
| Frescolat ® ML cryst | Menthyl Lactate | 0.5 |
| N-Ethyl-p-menthane-3- | Ethyl Menthane Carboxamide | 0.2 |
| carboxamide | | |
| (Cooling Agent WS-3) | | |
| 1,2-Heptanediol, | 1,2-Heptanediol, | 0.5 |
| 2,3-Heptanediol | 2,3-Heptanediol | |
| (w/w % 95:5) | | |
| 2,3-Nonanediol | 2,3-Nonanediol | 0.05 |

TABLE 48
Frosty Facial Foam

| Ingredients | EU INCI | Amount |
|---|---|---|
| Aqua/Water | Aqua | ad 100 |
| Edeta B Powder | Tetrasodium EDTA | 0.1 |
| Glycerin | Glycerin | 3.0 |
| Potassium Hydroxide, | Potassium hydroxide | 3.5 |
| 85% Pellets | | |
| Myristic Acid 99% | Myristic acid | 6.0 |
| Lauric Acid, 99% | Lauric acid | 6.0 |
| Stearic Acid, 98% | Stearic acid | 11.0 |
| Miranol ® C2M CONC NP | Disodium Cocoamphodiacetate | 4.0 |
| SymMollient ® W/S | Trideceth-9, PEG-5 | 2.0 |
| | Isononanoate, Aqua | |
| Perfume oil PO1, PO2, | Parfum | 0.3 |
| PO3, PO4, or PO5 | | |
| Frescolat ® MGA Plus | Menthone Glycerin acetal, | 0.5 |
| | Menthol | |
| 1,2-Heptanediol/ | 1,2-Heptanediol/2,3-Heptanediol | 1.0 |
| 2,3-Heptanediol | | |
| (w/w % 95:5) | | |
| 1,2-Nonanediol | 1,2-Nonanediol | 0.3 |
| SymOcide ® PS | Phenoxyethanol, | 0.8 |
| | Decylene Glycol, | |
| | 1,2-Hexanediol | |
| Colour, 0.1% aq. | CI42090 | 0.5 |

TABLE 49
Fresh Hair Antidandruff shampoo

| Ingredients | EU INCI | Amount |
|---|---|---|
| Aqua | Aqua | ad 100 |
| EDTA B Powder | Disodium EDTA | 0.1 |
| Carbopol ® Aqua SF-1 | Acrylates copolymer | 11.0 |
| Polymer | | |
| Texapon ® NSO UP | Sodium Laureth sulfate | 35.0 |
| Dehyton ® K | Cocamidopropyl betaine | 8.0 |
| Plantacare ® 2000 UP | Decyl Glucoside | 4.0 |
| Perfume oil PO1, PO2, | Parfum | 1.0 |
| PO3, PO4, or PO5 | | |
| SymSave ® H | Hydroxyacetophenone | 0.5 |
| Hydrolite ® 6 | 1,2-Hexanediol | 1.0 |
| 1,2-Heptanediol | 1,2-Heptanediol | 0.95 |
| 2,3-Heptanediol | 2,3-Heptanediol | 0.05 |
| SymClariol ® | Decylene Glycol | 0.5 |
| Frescolat ® ML cryst. | Menthyl Lactate | 1.0 |
| SymCalmin ® | Pentylene Glycol, Butylene Glycol, | 0.2 |
| | Hydroxyphenyl propamidobenzoic | |
| | acid | |
| Crinipan ® AD | Climbazole | 0.3 |
| Crinipan ® PMC Green | Propanediol Caprylate | 1.0 |
| Propylene Glycol | Propylene Glycol | 2.0 |
| SymHair ® Shield | Pentylene Glycol, Aqua, Glycerin, | 1.0 |
| | Triticum vulgare bran extract, 1,2- | |
| | Hexanediol, Capryly glycol | |
| Sodium Hydroxide 10% | Aqua, Sodium hydroxide | 2.2 |
| solution | | |
| Actipone ® Alpha-Pulp | Aqua, Butylene Glycol, Malic acid, | 0.2 |
| | Actinidia chinensis fruit extract, | |
| | citrus aurantium dulcis juice, citrus | |
| | paradise juice, pyrus malus juice | |

TABLE 50
Anti-Oil Shampoo

| Ingredients | INCI | Amount |
|---|---|---|
| Aqua/Water | Aqua | ad 100 |
| SymSave ® H | Hydroxyacetophenone | 0.5 |
| Ucare ™ Polymer JR-400 | Polyquaternium-10 | 0.25 |
| Dehyton ® K | Cocamidopropyl Betaine | 10.0 |
| SymDiol ® 68 | 1,2-Hexanediol, Caprylyl Glycol | 0.5 |
| 1,2-Heptanediol | 1,2-Heptanediol | 0.4 |
| 2,3-Heptanediol | 2,3-Heptanediol | 0.02 |
| 1,2-Nonanediol | 1,2-Nonanediol | 0.3 |
| 2,3-Nonandiol | 2,3-Nonanediol | 0.01 |
| Texapon HBN | Sodium Laureth Sulfate | 30.0 |
| Amille GCK-12H | Potassium cocoyl Glycinate (and) | 6.0 |
| | Potassium cocoate (and) Aqua | |
| Rewopal ® PEG 6000DS | PEG-150 Distearate | 3.0 |
| Frescolat ® ML | Menthyl Lactate | 0.2 |
| SymControl ® Scalp | Water (Aqua), Glycerin, Mannitol, | 2.0 |
| | Tetraselmis Suecica Extract | |
| SymMollient ® W/S | Trideceth-9, PEG-5-Isononanoate, | 2.0 |
| | Aqua | |
| Cremophor ® CO 40 | PEG-40 Hydrogenated Castor oil | 1.0 |
| Surfactant | | |
| Crinipan ® PMC Green | Propanediol Caprylate | 0.5 |
| Cosmetic Colour Blue | CI 42090 | 0.33 |
| 1% sol. | | |
| Food Colour Tartrazine | CI 19140 | 0.12 |
| 0.5% sol. | | |
| Perfume oil PO1, PO2, | Parfum | 1.0 |
| PO3, PO4, or PO5 | | |
| Extrapone ® Green | Glycerin, Aqua, Camellia | 0.1 |
| Tea GW | sinensis Leaf Extract | |
| Citric Acid 30% sol. | Aqua, Citric acid | 0.15 |

TABLE 51
Detox Refreshing Spray for Hair

| Ingredients | INCI | Amount |
|---|---|---|
| Frescolat ® ML | Menthyl Lactate | 0.5 |
| N-Ethyl-p-menthane-3- | Ethyl Menthane Carboxamide | 0.4 |
| carboxamide | | |
| (Cooling Agent WS-3) | | |
| 1,2-Nonanediol, | 1,2-Nonanediol, 2,3-Nonanediol | 1.0 |
| 2,3-Nonanediol | | |
| (95:5 w/w %) | | |
| Perfume oil PO1, PO2, | Parfum | 1.0 |
| PO3, PO4, or PO5 | | |
| Solubilizer | PEG-40 Hydrogenated castor oil, | 6.0 |
| | Trideceth.9, Propylene Glycol, | |
| | Aqua | |
| Aqua/Water | Aqua | ad 100 |
| SymOcide ® PS | Phenoxyethanol, Decylene Glycol, | 1.0 |
| | 1,2-Hexanediol | |
| Hydrovition ® PLUS | Aqua, Pentylene Glycol, Glycerin, | 2.0 |
| 2290 | Fructose, Urea, citric Acid, sodium | |
| | hydroxide, Maltose, Sodium PCA, | |
| | Sodium chloride, Sodium Lactate, | |
| | Trehalose, Allantoin, Sodium | |
| | Hyaluronate, Glucose | |
| Dragosine ® | Carnosine | 0.2 |
| SymHair ® Shield | Pentylene Glycol, Aqua, Glycerin, | 1.0 |
| | Triticum vulgare bran extract, 1,2- | |
| | Hexanediol, Caprylyl Glycol | |
| Citric Acid 30% sol. | Aqua, Citric Acid | 0.1 |

TABLE 52
Scalp soothing hair conditioner with
UV-B/UV-A protection, rinse off

| Ingredients | INCI | Amount |
|---|---|---|
| SymLite ® G8 | Glyceryl caprylate | 0.2 |
| Frescolat ® Plus | Menthol, Menthyl Lactate | 0.3 |
| Crinipan ® PMC green | Propanediol caprylate | 0.5 |
| 1,2-Heptanediol | 1,2-Heptanediol | 0.5 |
| Abil ® 350 | Dimethicone | 0.1 |
| Dehyquart ® A CA | Cetrimonium Chloride | 0.5 |
| Dehyquart ® SP | Quaternium-52 | 4.0 |
| Dracorin ® CE | Glyceryl Stearate Citrate | 1.0 |
| EDETA BD | Disodium EDTA | 0.1 |
| Extrapone ® Green | Glycerin, Water (Aqua), Camellia | 0.7 |
| Tea GW | Sinensis Leaf Extract | |
| Perfume oil PO1, PO2, | Parfum | 0.5 |
| PO3, PO4, or PO5 | | |
| Lara Care ® A-200 | Galactoarabinan | 0.5 |
| Neutral Oil | Caprylic/Capric Triglyceride | 1.0 |
| PCL-Liquid ® 100 | Cetearyl Ethylhexoate | 0.3 |
| PCL Solid | Stearyl Heptanoate, Stearyl | 3.0 |
| | Caprylate | |
| SymOcide ® PS | Phenoxyethanol, Decylene | 1.0 |
| | Glycol, 1,2-Hexanediol | |
| Water (demineralized) | Water (Aqua) | ad 100 |

TABLE 53
Hair Soap SHAMPOO Bar

| Ingredients | EU INCI | Amount |
|---|---|---|
| Talco | Talc | ad 100 |
| Frescolat ® ML | Menthyl Lactate | 0.7 |
| Texapon ® ZACD | Sodium Lauryl Sulfate | 10.00 |
| SymOleo ® Vita7 | Glycine soja oil, Gossypium | 2.00 |
| | herbaceum seed oil, mangifera | |
| | idica seed butter, olea europaea | |
| | fruitoil, persea gratissima oil, | |
| | prununs amygdalus dulcis (sweet | |
| | almond) oil, Theobroma cacao | |
| | seed butter | |
| Cetiol ® SB 45 | Butyrospermum parkii butter | 3.00 |
| Mackamide ® CMA | Cocamide Mea | 4.00 |
| SymSol ® PF-3 | Aqua, Pentylene Glycol, Sodium | 5.00 |
| | Lauryl sulfoacetate, sodium oleoyl | |
| | sarcosinate, Sodium Chloride, | |
| | Sodium Oleate | |
| Food Colour Beta Carotene | Helianthus annuus (Sunflower) | 0.45 |
| E160A OilSoluble | seed oil, Beta-Carotene (CI40800) | |
| 1,2-Nonanediol | 1,2-Nonanediol | 0.3 |
| 2,3-Nonanediol | 2,3-Nonanediol | 0.05 |
| SymOcide ® PH | Phenoxyethanol, | 1.00 |
| | Hydroxyacetophenone, Caprylyl | |
| | Glycol, Aqua | |
| SymHair ® Restore | Glycerin, Triticum vulgare | 1.00 |
| | protein, Aqua | |
| Perfume oil PO1, PO2, | Parfum | 1.50 |
| PO3, PO4, or PO5 | | |

TABLE 54
Hair Refresher Mist

| Ingredients | EU INCI | Amount |
|---|---|---|
| SymDeo ® Plus | Lauryl alcohol, Phenoxyethanol, | 0.5 |
| | 2-Benzyleptanol, Decylene Glycol | |
| Perfume oil PO1, PO2, | Parfum | 0.1 |
| PO3, PO4, or PO5 | | |
| Frescolat ® ML nat. | Mentyl Lactate | 0.3 |
| Crinipan ® PMC green | Propanediol Caprylate | 0.3 |
| Solubilizer | PEG-40 Hydrogenated Castor oil, | 5.0 |
| | Trideceth-9, Propylene glycol, | |
| | Aqua | |
| SymCalmin ® | Pentylene Glycol, Butylene Glycol, | 0.2 |
| | Hydroxyphenyl Propamidobenzoic | |
| | acid | |
| SymControl ® Scalp | Water (Aqua), Glycerin, Mannitol, | 2.0 |
| | Tetraselmis Suecica Extract | |
| Genuvisco ® CG-131 | Carrageenan | 0.2 |
| 1,2-Heptanediol, | 1,2-Heptanediol, 2,3-Heptanediol | 0.5 |
| 2,3-Heptanediol | | |
| 2,3-Nonanediol | 2,3-Nonanediol | 0.025 |
| Propylene Glycol | Propylene Glycol | 3.0 |
| Butylene Glycol | Butylene Glycol | 3.0 |
| Aqua | Aqua | ad 100 |
| Edeta ® BD | Disodium EDTA | 0.1 |
| Sodium Benzoate | Sodium benzoate | 0.4 |
| Colour | | 0.5 |
| Actipone ® Rosemary | Glycerin, Aqua, Rosmarinus | 0.5 |
| GW | officinalis leaf extract | |

TABLE 55
Refreshing conditioning Foam

| Ingredients | EU INCI | Amount |
|---|---|---|
| Aqua/Water | Aqua | ad 100 |
| Edeta BD | Disodium EDTA | 0.05 |
| SymSave ® H | Hydorxyacetophenone | 0.5 |
| Solubilizer | PEG-40 Hydrogenated Castor oil, | 1.5 |
| | Trideceth-9, Propylene Glycol, | |
| | Aqua | |
| Hydrolite ® 5 green | Pentylene Glycol | 0.5 |
| 1,2-Nonanediol, 2,3- | 1,2-Nonanediol, 2,3-Nonanediol | 0.5 |
| Nonanediol (95:5 w/w %) | | |
| Frescolat ® Plus | Menthol, Menthyl Lactate | 0.5 |
| 3-((—)-Menthoxy) | 3-((—)-Menthoxy) | 0.1 |
| propane-1,2-diol | propane-1,2-diol | |
| SymSol ® PF-3 | Aqua, Pentylene Glycoll, Sodium | 5.0 |
| | Lauryl Sulfoacetate, Sodium | |
| | Oleoyl Sarcosinate, Sodium | |
| | Chloride, Sodium Oleate | |
| Perfume oil PO1, PO2, | Parfum | 1.0 |
| PO3, PO4, or PO5 | | |
| SymCalmin ® | Pentylene Glycol, Butylene Glycol, | 2.0 |
| | Hydroxyphenyl Propamidobenzoic | |
| | acid | |
| Citric Acid 50 % sol. | Aqua, Citric Acid | 0.05 |

TABLE 56
Virgin Mojito Pre-Shampoo

| Ingredients | EU INCI | Amount |
|---|---|---|
| Aqua/Water | Aqua | ad 100 |
| Nativacare ™ 5600 | Zea (Mays) Corn Starch | 1.0 |
| Keltrol ®-CG- SFT | Xanthan Gum | 0.5 |
| Glycerin | Glycerin | 2.0 |
| SymSave ® H | Hydroxyacetophenone | 0.5 |
| Hydrolite ® 5 green | Pentylene Glycol | 3.0 |
| Solubilizer | PEG-40 Hydrogenated castor oil, | 3.0 |
| | Trideceth-9, Propylene Glycol, | |
| | Aqua | |
| Oxynex ® ST Liquid | Diethylhexyl | 0.1 |
| | Syringylidenemalonate, | |
| | Caprylic/capric triglyeride | |
| Frescolat ® Plus | Menthol, Menthyl Lactate | 1.0 |
| Frescolat ® MGA | Menthone Glycerin Acetal | 0.6 |
| Perfume oil PO1, PO2, | Parfum | 0.5 |
| PO3, PO4, or PO5 | | |
| 1,2-Heptanediol, 2,3- | 1,2-Heptanediol, | 0.3 |
| Heptanediol (w/w % 95:5) | 2,3-Heptanediol | |
| 2,3-Nonanediol | 2,3-Nonanediol | 0.025 |
| SymReboot ™ L19 | Maltodextrin, Lactobacillus | 0.5 |
| | ferment | |
| Extrapone ® Lime | Propylene Glycol, Aqua, Citrus | 1.0 |
| | aurantifolia juice | |
| Colour I: FD&C Yellow | Aqua, CI19140, Sodium | 0.8 |
| no. 5 −0.01% solution | Chloride, Sodium Sulfate | |
| Colour II: FD&C Blue | Aqua, CI42090, Sodium | 0.4 |
| no. 1 −0.01% solution | Chloride, Sodium Sulfate | |
| Sodium Hydroxide | Aqua, Sodium Hydroxide | 0.2 |
| 10% solution | | |

TABLE 57
Anti-itch hair conditioner, leave on

| Ingredients | INCI | Amount |
|---|---|---|
| SymLite ® G8 | Glyceryl caprylate | 0.15 |
| 1,2-Heptanediol | 1,2-Heptanediol | 0.5 |
| 1,2-Nonanediol | 1,2-Nonanediol | 0.05 |
| Frescolat ® X-cool | Menthyl Ethylamido Oxalate | 0.6 |
| Dragosantol ® 100 | Bisabolol | 0.1 |
| Dehyquart ® A CA | Cetrimonium Chloride | 0.5 |
| Dehyquart ® SP | Quaternium-52 | 4.0 |
| Dracorin ® CE | Glyceryl Stearate Citrate | 1.0 |
| Drago-Oat-Active | Water (Aqua), Butylene Glycol, | 2.0 |
| | Avena Sativa (Oat) Kernel Extract | |
| Perfume oil PO1, PO2, | Parfum | 0.1 |
| PO3, PO4, or PO5 | | |
| Lara Care ® A-200 | Galactoarabinan | 1.5 |
| Neutral Oil | Caprylic/Capric Triglyceride | 1.0 |
| PCL Liquid ® 100 | Cetearyl Ethylhexoate | 0.3 |
| Polymer JR 400 | Polyquaternium-10 | 0.1 |
| Propylene Glycol | Propylene Glycol | 0.8 |
| SymGlucan ® | Aqua, Glycerin, 1,2-Hexandiol, | 5 |
| | Caprylyl Glycol, Beta-Glucan | |
| SymMollient ® W/S | Trideceth-9, PEG-5 Isononanoate, | 2.0 |
| | Water (Aqua) | |
| SymOcide ® PH | Phenoxyethanol, | 1.2 |
| | Hydroxyacetophenone, Caprylyl | |
| | Glycol, Aqua | |
| SymSol ®PF3 | Water, Pentylene Glycol, Sodium | 1.5 |
| | Lauryl Sulfoacetate, Sodium | |
| | Oleoyl Sarcosinate, Sodium | |
| | Chloride, Disodium Sulfoacetate, | |
| | Sodium Oleate, Sodium Sulfate | |
| Water, demineralized | Water (Aqua) | ad 100 |

TABLE 58
Antidandruff Shampoo

| Ingredients | INCI | Amount |
|---|---|---|
| Water, distilled | Water (Aqua) | ad 100 |
| Polymer JR 400 | Polyquaternium-10 | 0.4 |
| Texapon ® NSO IS | Sodium Laureth Sulfate | 40.0 |
| Farnesol | Farnesol | 0.1 |
| Dehyton K | Cocamidopropyl Betaine | 8.0 |
| Plantacare 2000 UP | Decyl Glucoside | 2.0 |
| Sodium Methylparaben | Sodium Methylparaben | 0.3 |
| Citric Acid 10% solution | Citric Acid | 0.8 |
| Frescolat ® X-cool | Menthyl Ethylamido Oxalate | 0.5 |
| Sym Triol ® | Caprylyl Glycol, 1,2-Hexanediol, | 0.8 |
| | Methylbenzyl Alcohol | |
| Crinipan ® AD | Climbazole | 0.5 |
| SymMollient ® W/S | Trideceth-9, PEG-5 | 2.0 |
| | Isononanoate, Water (Aqua) | |
| Tinogard ® Q | Tri(tetramethylhydroxy- | 0.02 |
| | piperodonol) Citrate | |
| Sodium Chloride | Sodium Chloride | 0.5 |
| Colour I | Colour | 0.3 |
| Colour II | Colour | 0.1 |
| Perfume oil PO1, PO2, | Perfume | 0.4 |
| PO3, PO4 or PO5 | | |
| Octopirox | Octopirox | 0.1 |
| 1,2-Heptanediol | 1,2-Heptanediol | 0.4 |
| 2,3-Heptanediol | 2,3-Heptanediol | 0.05 |
| 1,2-Octanediol | 1,2-Octanediol | 0.2 |
| 2,3-Octanediol | 2,3-Octanediol | 0.01 |

TABLE 59
Low Cost-Refreshing Body Lotion

| Ingredients | EU INCI | Amount |
|---|---|---|
| Aqua/Water | Aqua | ad 100 |
| Hydrovition ® | Aqua, Pentylene Glycol, Glycerin, | 2.0 |
| PLUS 2290 | Fructose, Urea, Citric acid, | |
| | Sodium Hydroxide, Maltose, | |
| | Sodium PCA, Sodium chloride, | |
| | Sodium Lactate, Trehalose, | |
| | Allantoin, Sodium Hyaluronate, | |
| | Glucose | |
| Extrapone ® | Aqua, Propylene Glycol, Butylene | 1.0 |
| Camomile | Glycol, Chamomilla recutita, | |
| | (Matricaria) Flower extract, | |
| | Bisabolol | |
| Amaze ® XT | Dehydroxanthan Gum | 0.7 |
| Avicel ® PC591 | Cellulose, Cellulose Gum | 0.3 |
| Caprylic/Capric | Caprylic/Capric Triglyceride | 5.0 |
| Triglyceride | | |
| Dragoxat ® 89 | Ethylhexyl Isononanoate | 3.0 |
| Dracorin ® GOC | Glyceryl Oleate Citrate, | 0.3 |
| | CaprylicCapric Triglyceride | |
| Ethanol 96 % | Alcohol denat. | 5.0 |
| Frescolat ® ML | Menthyl Lactate | 0.3 |
| Perfume oil PO1, PO2, | Parfum | 0.2 |
| PO3, PO4, or PO5 | | |
| Sodium Hydroxide | Aqua, Sodium hydroxide | 0.1 |
| 10% solution | | |
| 1,2-Heptanediol | 1,2-Heptanediol | 1.5 |
| 2,3-Heptanediol | 2,3-Heptanediol | 0.05 |
| 1,2-Nonanediol | 1,2-Nonanediol | 0.2 |
| 2,3-Nonandiol | 2,3-Nonanediol | 0.01 |
| SymOcide PS | Phenoxyethanol, Decylene Glycol, | 1.0 |
| | 1,2-Hexanediol | |

TABLE 60
Intensive Care, Detox& Repair Hand Cream 2-in-1

| Ingredients | EU INCI | Amount |
|---|---|---|
| Emulsiphos ® | Potassium cetyl phosphate, | 1.50 |
| | hydrogenated palm glycerides | |
| Lanette ® O | Cetearyl alcohol | 1.50 |
| Beeswax | Beeswax | 1.00 |
| PCL-Liquid ®100 | Cetearyl Ethylhexanoate | 3.00 |
| Softisan ®100 | Hydrogenated coco-glycerides | 5.00 |
| PCL-Solid | Stearyl Heptanoate, Stearyl | 5.00 |
| | Caprylate | |
| Caprylic/Capric | Caprylic/Capric Triglyceride | 1.50 |
| Triglyceride | | |
| Tegosoft ® MM | Myristyl myristate | 2.50 |
| SymDetox ® 1711 | 3,3,5-Trimethylcyclohexyl | 0.50 |
| | succinate dimethylamide | |
| SymRepair ® 100 | Hexyldecanol, Bisabolol, | 1.00 |
| | Cethylhydroxyproline palmitamide, | |
| | stearic acid, Brassica campestris | |
| | (Rapeseed) sterols | |
| SymDecanox ™ HA | Caprylic/Capric Triglyceride, | 1.00 |
| | Hydroxymethoxyphenyl decanone | |
| Cosmedia ® SP | Sodium Polyacrylate | 0.60 |
| Aristoflex ® AVC | Ammonium | 0.60 |
| | Acryloyoldimethyltaurate/VP | |
| | Copolymer | |
| Aqua/Water | Aqua | ad 100 |
| Edeta ® BD | Disodium Edta | 0.10 |
| Frescolat ® X-cool | Menthyl Ethylamido Oxalate | 0.30 |
| SymSave ® H | Hydroxyacetophenone | 0.50 |
| Hydroviton ® | Aqua, Pentylene Glycol, Glycerin, | 2.00 |
| PLUS 2290 | Fructose, Urea, Citric acid, | |
| | Sodium hydroxide, Maltose, | |
| | Sodium PCA, Sodium chloride, | |
| | Sodium Lactate, Trehalose, | |
| | Allantoin, Sodium Hyaluronate, | |
| | Glucose | |
| SymEssence ® | Chamomilla recutita (Matricaria) | 1.00 |
| Camomile CC | Flower/Leaf/Stem water | |
| Colour | | 0.80 |
| Tinogard ® Q | Tris(tetramethylhydroxy- | 0.05 |
| | piperidino) citrate | |
| Perfume oil PO1, PO2, | Parfum | 0.40 |
| PO3, PO4, or PO5 | | |
| Sensocel ® 10 | Cellulose | 2.00 |
| Dipropylene glycol | Dipropylene Glycol | 0.80 |
| Retinyl Palmitate | Retinyl Palmitate | 0.10 |
| 1,2-Heptanediol, | 1,2-Heptanediol, 2,3-Heptanediol | 1.0 |
| 2,3-Heptanediol | | |
| (w/w % 95:5) | | |
| 1,2-Nonanediol | 1,2-Nonanediol | 0.1 |
| SymOcide ® C | o-Cymen-5-ol | 0.10 |

TABLE 61
Hygiene & Care Hand Cream

| Ingredients | EU INCI | Amount |
|---|---|---|
| Emulsiphos ® | Potassium Cetyl Phosphate, | 1.5 |
| | Hydrogenated Palm Glycerides | |
| PCL-Solid | Stearyl Heptanoate, Stearyl | 2.5 |
| | Caprylate | |
| Dragoxat ® 89 | Ethylhexyl Isononanoate | 1.5 |
| Caprylic/Capric | Caprylic/Capric Triglyceride | 2.0 |
| Triglyceride | | |
| Tegosoft ® MM | Myristyl Myristate | 2.5 |
| SymDecanox ® HA | Caprylic/Capric Triglyceride, | 1.0 |
| | Hydroxymethoxyphenyl decanone | |
| Frescolat ® X-Cool | Menthyl ethylamido oxalate | 0.5 |
| Frescolat ® Plus | Menthol, Menthyl Lactate | 0.6 |
| SymCare ® O | Hexyldecanol, Pentylene Glycol, | 2.0 |
| | 4-t-Butylcylohexanol, Bisabolol, | |
| | Cetylhydroxyproline Palmitamide, | |
| | Hydroxyphenyl Propamidobenzoic | |
| | acid, Stearic acid, Brassica | |
| | campestris (Rapeseed)Sterols, | |
| | Zingiber officinale (Ginger)Root | |
| | extract | |
| Beeswax | Beeswax | 1.0 |
| Cosmedia ® SP | Sodium Polyacrylate | 0.8 |
| Aristoflex ® AVC | Ammonium | 0.6 |
| | Acryloydimethyltaurate/VP | |
| | Copolymer | |
| Aqua/Water | Aqua | ad 100 |
| Edeta BD | Disodium EDTA | 0.1 |
| SymSave ® H | Hydroxyacetophenone | 0.5 |
| Hydroviton ® PLUS 2290 | Aqua, Pentylene Glycol, Glycerin, | 2.0 |
| | Fructose, Urea, Citric acid, | |
| | Sodium hydroxide, Maltose, | |
| | Sodium PCA, Sodium chloride, | |
| | Sodium Lactate, Trehalose, | |
| | Allantoin, Sodium Hyaluronate, | |
| | Glucose | |
| Actipone Stinging | Glycerin, Urtica Dioica Juice, | 1.0 |
| Nettle Juice | Aqua | |
| SymEssence ® | Chamomilla Recuitta (Matricaria), | 1.0 |
| Camomile CC | Flower/Leaf/Stem Water | |
| Colour | | 0.9 |
| Tinogard ® Q | Tris(Tetramethylhydroxy- | 0.05 |
| | piperidinol) Citrate | |
| 1,2-Heptanedil, | 1,2-Heptanediol, 2,3-Heptanediol | 0.6 |
| 2,3-Heptanediol | | |
| (95:5 w/w %) | | |
| 1,2-Nonanediol | 1,2-Nonanediol | 0.2 |
| Perfume oil P1, P2, | Parfum | 0.4 |
| P3, P4 or P5 | | |
| SymOcide ® C | o-Cymen-5-ol | 0.1 |
| SymDeo ® Plus | Lauryl alcohol, Phenoxyethanol, 2- | 0.5 |
| | Benzylheptanol, Decylene Glycol | |

TABLE 62
Refreshing and Soothing After Sun Cream

| Ingredients | EU INCI | Amount |
|---|---|---|
| Dracorin ® CE | Glyceryl Stearate Citrate | 2.5 |
| Cutina ® GMS V | Glyceryl Stearate | 2.5 |
| Coconut Oil | Cocos Nucifera Oil | 3.0 |
| SymMollient ® PDCC | Propanediol Dicaprylate/Caprate | 8.0 |
| Plantoil Triglyceride | Caprylic/Capric Triglyceride | 4.0 |
| 1,2-Heptandiol, | 1,2-Heptanediol, 2,3-Heptanediol | 1.0 |
| 2,3-Heptanediol | | |
| (w/w % 95:5) | | |
| 1,2-Nonanediol | 1,2-Nonanediol | 0.1 |
| Tocopherol | Tocopherol | 0.05 |
| Demin. Water | Aqua | ad100 |
| Glycerin | Glycerin | 2.0 |
| SymOcide ® PS | Phenoxyethanol, Decylene Glycol, | 1.0 |
| | 1,2-Hexanediol | |
| Nativacare ™ 8600 | Oryza Sativa (Rice) Starch | 1.0 |
| Keltrol ® CG-SFT | Xanthan Gum | 0.4 |
| Sirhamnose | Silanetriol (and) Rhamnose | 3.0 |
| D-Panthenol 75 L | Panthenol | 1.0 |
| Frescolat ® ML nat. | Menthyl Lactate | 1.5 |
| Perfume oil PO1, PO2, | Parfum | 0.3 |
| PO3, PO4, or PO5 | | |

TABLE 63
Daily Well Aging, with SPF 30, UVA/UVB balanced, w/o Octocrylene

| Raw material | INCI | Amount |
|---|---|---|
| Lanette ® O | Cetearyl alcohol | 1.5 |
| Emulsiphos ® | Potassium cetyl phosphate | 2.5 |
| | Hydrogenated palm glycerides | |
| SymUrban ® | Benzylidene | 0.3 |
| | dimethoxydimethylindanone | |
| Neo Heliopan ® 357 | Butyl methoxydibenzoylmethane | 4.0 |
| Neo Heliopan ® OS | Ethylhexyl salicylate | 5.0 |
| Neo Heliopan ® BMT | Bis-ethylhexyloxyphenol | 3.0 |
| | methoxyphenyl triazine | |
| Uvasorb ® HEB | Diethylhexyl butamido triazone | 2.0 |
| SymMollient ® S | Cetearyl nonanoate | 1.0 |
| Cetiol ® Sensoft | Propylheptyl caprylate | 4.0 |
| Tegosoft ® TN | C12-15 alkyl benzoate | 11.0 |
| SymDecanox ™ HA | Caprylic/capric triglyceride | 1.0 |
| | Hydroxymethoxyphenyl decanone | |
| Isoadipate | Diisopropyl adipate | 7.5 |
| EDTA ® BD | Disodium edta | 0.1 |
| Carbopol ® ETD | Carbomer | 0.3 |
| 2050 Polymer | | |
| Keltrol ® CG-T | Xanthan gum | 0.2 |
| Aqua/Water | Aqua | ad 100 |
| 1,2-Heptanediol | 1,2-Heptanediol | 0.7 |
| 2,3-Heptanediol | 2,3-Heptanediol | 0.3 |
| Glycerin 99.5% | Glycerin, Aqua | 1.0 |
| Lanette ® E | Sodium cetearyl sulfate | 0.8 |
| Neo Heliopan ® Hydro | Phenylbenzimidazole sulfonic acid | 3.0 |
| Biotive ® L-Arginine | Arginine | 1.5 |
| Propylene Glycol | Propylene glycol | 3.0 |
| Sodium Hydroxide | Aqua, Sodium hydroxide | 2.0 |
| 10% solution | | |
| SymSave ® H | Hydroxyacetophenone | 0.6 |
| Phenoxyethanol | Phenoxyethanol | 0.5 |
| Dragosine ® | Carnosine | 0.2 |
| SymVital ® AR | Zingiber officinale (ginger) root | 0.2 |
| | extract | |
| Frescolat ® ML Cryst. | Menthyl lactate | 0.5 |
| Perfume oil PO1; PO2; | Parfum | 1.0 |
| PO3; PO4; or PO5 | | |
| Tapioca Pure | Tapioca starch | 2.0 |

TABLE 64
Mosquito & tick repellent Spray

| Tradename | INCI | w/w % |
|---|---|---|
| Ethanol | Ethanol | ad 100 |
| DEET | Diethyl Toluamide | 20.0 |
| Citriodiol | Cyclized Eucalyptus Citriodora | 3.0 |
| | Leaf/Twig Oil | |
| Perfume Oil PO1, PO2, | Fragrance | 0.2 |
| PO3, PO4, or PO5 | | |
| 1,2-Heptanediol | 1,2-Heptanediol | 1.5 |
| 2,3-Heptanediol | 2,3-Heptanediol | 0.02 |
| 1,2-Octanediol | Caprylyl Glycol | 0.2 |
| 2,3-Octanediol | 2,3-oOtanediol | 0.01 |

TABLE 65
Insect Repellent Spray. aqueous/ethanolic

| Tradename | INCI | w/w % |
|---|---|---|
| Ethanol | Ethanol | 60.0 |
| Water | Water (Aqua) | 20.0 |
| Perfume Oil PO1, PO2, | Fragrance | 0.3 |
| PO3, PO4, or PO5 | | |
| Icaridin | Hydroxyethyl Isobutyl Piperidine | 20.0 |
| | Carboxylate | |
| Eucalyptus Oil | Eucalyptus essential oil | 0.3 |
| | Eucalyptus Oil | |
| SymSol PF-3 | Water (Aqua), Sodium Lauryl | 1.5 |
| | Sulfoacetate, Pentylene Glycol, | |
| | Sodium Oleoyl Sarcosinate, | |
| | Sodium Chloride, Sodium Oleate | |
| 1,2-Heptanediol | 1,2-Heptanediol | 1.0 |
| 2,3-Heptanediol | 2,3-Heptanediol | 0.01 |
| 1,2-Octanediol | Caprylyl Glycol | 0.3 |
| 2,3-Octanediol | 2,3-Octanediol | 0.05 |

TABLE 66
Insect Repellent Solution for Wipes

| Tradename | INCI | w/w % |
|---|---|---|
| Arlamol E | PPG-15 Stearylether | 3.0 |
| Polyethylenglycol 400 | PEG-8 | 5.0 |
| Ethanol | Ethanol | 35.0 |
| Glycerin | Glycerin | 4.0 |
| IR3535 | Etyhl Butylacetylamino | 20.0 |
| | propionate | |
| Citronellol | Citronellol | 0.5 |
| Perfume Oil PO1, PO2, | Fragrance | 0.3 |
| PO3, PO4, or PO5 | | |
| Solubilizer | Trideceth-9 PEG 40, | 2.5 |
| | Hydrogenated Castor Oil | |
| Water | Water (Aqua) | ad 100 |
| 1,2-Heptanediol | 1,2-Heptanediol | 1.0 |
| 2,3-Heptanediol | 2,3-Heptanediol | 0.01 |
| 1,2-Octanediol | Caprylyl Glycol | 0.3 |
| 2,3-Octanediol | 2,3-Octanediol | 0.05 |

TABLE 67
Anti-Sweat Sunscreen Lotion with repellent

| Raw material | INCI | Amount |
|---|---|---|
| Aqua/Water | Aqua | ad 100 |
| 1,2-Heptanediol | 1,2-Heptanediol | 1.0 |
| 2,3-Heptanediol | 2,3-Heptanediol | 0.01 |
| 1,2-Octanediol | Caprylyl Glycol | 0.3 |
| 2,3-Octanediol | 2,3-Octanediol | 0.05 |
| Citric Acid 30% Sol. | Aqua, Citric acid | 0.6 |
| Aristoflex ® Velvet | Polyacrylate crosspolymer 11 | 0.6 |
| Glycerin 99.5% | Glycerin, Aqua | 2.0 |
| SymSol ® PF-3 | Aqua, Pentylene glycol, Sodium | 1.0 |
| | lauryl sulfoacetate, Sodium oleoyl | |
| | sarcosinate, Sodium chloride, | |
| | Sodium oleate | |
| SymSave ® H | Hydroxyacetophenone | 0.3 |
| Neo Heliopan ® 357 | Butyl methoxydibenzoylmethane | 3.0 |
| Neo Heliopan ® 303 | Octocrylene | 10.0 |
| Neo Heliopan ® OS | Ethylhexyl salicylate | 5.0 |
| Neo Heliopan ® HMS | Homosalate | 10.0 |
| Neo Heliopan ® E1000 | Isoamyl p-methoxycinnamate | 2.0 |
| Neo Heliopan ® BMT | Bis-ethylhexyloxyphenol | 3.0 |
| | methoxyphenyl triazine | |
| DEET | Diethyl Toluamide | 5.0 |
| Cpoconut Oil | Coconut Oil | 2.0 |
| Dragoxat ® 89 | Ethylhexyl isononanoate | 6.0 |
| SymMollient ® S | Cetearyl nonanoate | 1.0 |
| EDTA ® BD | Disodium edta | 0.1 |
| SymDecanox™ HA | Caprylic/capric triglyceride | 1.0 |
| | Hydroxymethoxyphenyl decanone | |
| Frescolat ® Plus | Menthol, Menthyl lactate | 0.3 |
| Citriodiol | Cyclized Eucalyptus Citriodora | 3.0 |
| | Leaf/Twig Oil | |
| N-Ethyl-p-menthane-3-carboxamide | Ethyl Menthane Carboxamide | 0.2 |
| (Cooling Agent WS-3) | | |
| SymDeo ® Plus | Lauryl alcohol, Phenoxyethanol, 2- | 1.0 |
| | benzylheptanol, Decylene glycol | |
| Hydroviton ® PLUS 2290 | Aqua, Pentylene glycol, Glycerin, | 4.0 |
| | Fructose, Urea, Citric acid, Sodium | |
| | hydroxide, Maltose, Sodium pca, | |
| | Sodium chloride, Sodium lactate, | |
| | Trehalose, Allantoin, Sodium | |
| | hyaluronate, Glucose | |
| Sensocel ® 10 | Cellulose | 2.0 |
| Perfume Oil PO1, PO2, PO3, PO4, or PO5 | Fragrance | 0.5 |

TABLE 68
Anti-Sweat Sunscreen Lotion

| Raw material | INCI | Amount |
|---|---|---|
| Aqua/Water | Aqua | ad 100 |
| 2,3-Nonanediol | 2,3-Nonanediol | 0.05 |
| 1,2-Nonanediol | 1,2-Nonanediol | 0.2 |
| 1,2-Heptanediol, 2,3-Heptanediol | 1,2-Heptanediol, 2,3-Heptanediol | 1.5 |
| (w/w % 97:3) | | |
| Citric Acid 30% Sol. | Aqua, Citric acid | 0.6 |
| Aristoflex ® Velvet | Polyacrylate crosspolymer 11 | 0.6 |
| Glycerin 99.5% | Glycerin, Aqua | 2.0 |
| SymSol ® PF-3 | Aqua, Pentylene glycol, Sodium | 1.0 |
| | lauryl sulfoacetate, Sodium oleoyl | |
| | sarcosinate, Sodium chloride, | |
| | Sodium oleate | |
| SymSave ® H | Hydroxyacetophenone | 0.3 |
| Neo Heliopan ® 357 | Butyl methoxydibenzoylmethane | 3.0 |
| Neo Heliopan ® 303 | Octocrylene | 10.0 |
| Neo Heliopan ® OS | Ethylhexyl salicylate | 5.0 |
| Neo Heliopan ® HMS | Homosalate | 10.0 |
| Neo Heliopan ® E1000 | Isoamyl p-methoxycinnamate | 2.0 |
| Neo Heliopan ® BMT | Bis-ethylhexyloxyphenol | 3.0 |
| | methoxyphenyl triazine | |
| Floraesters K-100 ® Jojoba | Hydrolyzed jojoba esters | 2.0 |
| | Jojoba esters | |
| | Aqua | |
| Dragoxat ® 89 | Ethylhexyl isononanoate | 6.0 |
| SymMollient ® S | Cetearyl nonanoate | 1.0 |
| EDTA ® BD | Disodium edta | 0.1 |
| SymDecanox ™ HA | Caprylic/capric triglyceride | 1.0 |
| | Hydroxymethoxyphenyl decanone | |
| Frescolat ® Plus | Menthol, Menthyl lactate | 0.3 |
| N-Ethyl-p-menthane-3-carboxamide | Ethyl Menthane Carboxamide | 0.2 |
| (Cooling Agent WS-3) | | |
| SymDeo ® Plus | Lauryl alcohol, Phenoxyethanol, | 1.0 |
| | 2-benzylheptanol, Decylene glycol | |
| Hydroviton ® PLUS 2290 | Aqua, Pentylene glycol, Glycerin, | 4.0 |
| | Fructose, Urea, Citric acid, | |
| | Sodium hydroxide, Maltose, | |
| | Sodium pca, Sodium chloride, | |
| | Sodium lactate, Trehalose, | |
| | Allantoin, Sodium hyaluronate, | |
| | Glucose | |
| Sensocel ® 10 | Cellulose | 2.0 |

TABLE 69
HH-Cream Haute Homme, with exp. SPF 15, UVA/UVB

| Raw material | INCI | Amount |
|---|---|---|
| Aqua/Water | Aqua | ad 100 |
| 2,3-Heptanediol | 2,3-Heptanediol | 0.5 |
| 1,2-Nonanediol, 2,3-Nonanediol | 1,2-Nonanediol, 2,3-Nonanediol | 0.5 |
| (95:5 w/w %) | | |
| SymSol ® PF-3 | Aqua, Pentylene glycol, Sodium | 2.0 |
| | lauryl sulfoacetate, Sodium oleoyl | |
| | sarcosinate, Sodium chloride, | |
| | Sodium oleate | |
| Neo Heliopan ® Hydro | Phenylbenzimidazole sulfonic acid | 2.5 |
| Biotive ® L-Arginine | Arginine | 1.0 |
| Sodium Hydroxide 10% solution | Aqua, Sodium hydroxide | 1.3 |
| Aqua Keep 10SH-NFC | Sodium acrylates crosspolymer-2 | 2.0 |
| SymSave ® H | Hydroxyacetophenone | 0.5 |
| Dragosine ® | Carnosine | 0.1 |
| Hydrolite ® 5 green | Pentylene glycol | 2.5 |
| Neo Heliopan ® OS | Ethylhexyl salicylate | 3.0 |
| Neo Heliopan ® 303 | Octocrylene | 3.0 |
| Neo Heliopan ® HMS | Homosalate | 3.0 |
| SymMollient ® S | Cetearyl nonanoate | 1.0 |
| Isoadipate | Diisopropyl adipate | 2.5 |
| SymRelief ® 100 | Bisabolol | 0.2 |
| | Zingiber officinale root extract | |
| SymDecanox ™ HA | Caprylic/capric triglyceride | 1.5 |
| | Hydroxymethoxyphenyl decanone | |
| Sym Detox ® | 3,3,5-Trimethylcyclohexyl | 0.5 |
| | succinate dimethylamide | |
| SymUrban ® | Benzylidene | 0.5 |
| | dimethoxydimethylindanone | |
| Edeta ® BD | Disodium edta | 0.1 |
| Frescolat ® X-Cool | Menthyl ethylamido oxalate | 0.5 |
| 3-((−)-Menthoxy)propane-1,2-diol | 3-((−)-Menthoxy)propane-1,2-diol | 0.3 |
| SymBright ™ 2036 | Sclareolide | 0.1 |
| SymWhite ® PLUS | Caprylic/capric triglyceride, | 1.3 |
| | Pentylene glycol | |
| | Phenylethyl resorcinol, Bisabolol, | |
| | Butyl methoxydibenzoylmethane | |
| Keltrol ® CG-BT | Xanthan gum | 0.2 |
| FOOD COLOUR BROWN | Titanium dioxides (CI 77891), Iron | 2.0 |
| E172 + E171 POWDER | oxides (CI 77492), Iron oxides | |
| | (CI77491), Iron oxides (CI 77499) | |
| Perfume oil PO1; PO2; PO3; PO4; or PO5 | Parfum | 0.5 |
| SymGlucan ® (151719) | Aqua, Glycerin, 1,2-Hexanediol, | 5.0 |
| | Caprylyl glycol, Beta-glucan | |

TABLE 70
BB Cream Dark Tone Spf-20 (Expected)

| Raw material | INCI | Amount |
|---|---|---|
| Emulsiphos ® | Potassium cetyl phosphate | 2.5 |
| | Hydrogenated palm glycerides | |
| Eumulgin ® SG | Sodium stearoyl glutamate | 0.5 |
| PCL-Liquid ® 100 | Cetearyl ethylhexanoate | 2.0 |
| Lanette ® O | Cetearyl alcohol | 1.0 |
| Dragoxat ® 89 | Ethylhexyl isononanoate | 1.0 |
| Cutina ® PES | Pentaerythrityl distearate | 1.0 |
| SymMollient ® S | Cetearyl nonanoate | 1.0 |
| Xiameter ® PMX-0245 | Cyclopentasiloxane | 2.0 |
| Cyclopentasiloxane | | |
| Xiameter ® PMX-200 Silicone Fluid 50 | Dimethicone | 1.0 |
| cs | | |
| Neo Heliopan ® 357 | Butyl methoxydibenzoylmethane | 3.0 |
| Neo Heliopan ® 303 | Octocrylene | 7.0 |
| Neo Heliopan ® HMS | Homosalate | 7.0 |
| Neo Heliopan ® OS | Ethylhexyl salicylate | 5.0 |
| SymDecanox ™ HA | Caprylic/capric triglyceride | 2.0 |
| | Hydroxymethoxyphenyl decanone | |
| Talcum | Talc | 2.0 |
| Food Colour Titanium Dioxide Powder | CI 77891 | 0.8 |
| E171 | | |
| Keltrol ® CG-SFT | Xanthan gum | 0.4 |
| Carbopol ® Ultrez 20 Polymer | Acrylates/c10-30 alkyl acrylate | 0.3 |
| | crosspolymer | |
| EDTA NA2 | Disodium EDTA | 0.2 |
| Aqua/Water | Aqua | ad 100 |
| Neo Heliopan ® Hydro | Phenylbenzimidazole sulfonic acid | 1.0 |
| Biotive ® L-Arginine | Arginine | 1.0 |
| Sodium Hydroxide 10% solution | Aqua | 0.7 |
| | Sodium hydroxide | |
| COSMETIC COLOUR BROWN | CI 77891 | 1.5 |
| POWDER | CI 77492 | |
| | CI 77491 | |
| | CI 77499 | |
| Food Colour Iron Oxide Yellow E172 | CI 77492 | 0.3 |
| Glycerin | Glycerin | 3.0 |
| Hydrolite ® 5 green | Pentylene glycol | 1.5 |
| Hydrolite ® CG | Caprylyl glycol | 0.2 |
| SymSave ® H | Hydroxyacetophenone | 0.5 |
| 1,2-Nonanediol | 1,2-Nonanediol | 0.1 |
| 1,2-Heptanediol, 2,3-Heptanediol | 1,2-Heptanediol, 2,3-Heptanediol | 0.5 |
| (w/w % 95:5) | | |
| Hydroviton ® PLUS 2290 | Aqua, Pentylene glycol, Glycerin, | 2.0 |
| | Fructose, Urea, Citric acid, | |
| | Sodium hydroxide, Maltose, | |
| | Sodium pca, Sodium chloride, | |
| | Sodium lactate, Trehalose, | |
| | Allantoin, Sodium hyaluronate, | |
| | Glucose | |
| Frescolat ® ML | Mentyl Lactate | 0.4 |
| Perfume oil PO1; PO2; PO3; PO4; or PO5 | Parfum | 0.5 |

TABLE 71
Refreshing Lip Gloss

| Raw material | INCI | Amount |
|---|---|---|
| Covaclear | Isononyl Isononanoate, | 19.7 |
| | Polybutenen, Pentaerythrityl | |
| | Tetraisostearate, Isostearyl | |
| | alcohol | |
| PCL-Solid | Stearyl Heptanoate, Stearyl | 8.0 |
| | Caprylate | |
| Isodragol | Triisononanoin | 7.0 |
| Dragoxat ® 89 | Ethylhexyl Isononanoate | 5.0 |
| Rewopal ® PIB1000 | Polyisobutene | 8.0 |
| Covapate Unired LC3703 | Ricinus Communis Seed oil, | 0.006 |
| | CI15850 | |
| Versagel ® ME750 | Hydrogenated polyisobutene, | ad 100 |
| | Ethylene/Propylene/Styrene | |
| | copolymer, | |
| | Butylene/Ethylene/Styrene | |
| | Copolymer | |
| Tocoperyl Acetate | Tocopheryl Acetate | 0.3 |
| Frescolat ® X-Cool | Menthyl Ethaylamido oxalate | 1.0 |
| Frescolat ® ML | Mentyl Lactate | 0.5 |
| 1,2-Heptanediol, | 1,2-Heptanediol, 2,3-Heptanediol | 0.6 |
| 2,3-Heptanediol | | |
| (95:5 w/w %) | | |
| 1,2-Nonanediol | 1,2-Nonanediol | 0.05 |
| SymRelief ® 100 | Bisabolol, Zingbier officinale root | 0.1 |
| | extract | |
| Aroma | Flavour | 0.1 |

TABLE 72
Cool Sebum Control Cleanser

| Raw material | INCI | Amount |
|---|---|---|
| Proteol ™ APL | Sodium cocoyl apple amino acids | 5.0 |
| Aqua/Water | Aqua | ad 100 |
| Neo-PCL Water Soluble N | Trideceth-9, PEG-5 | 15.0 |
| | Ethylhexanoate, Aqua | |
| Xiameter ® XM OFX-0193 Fluid | PEG-12 Dimethicone | 5.0 |
| Glycerin | Glyceirn | 8.0 |
| Perfume oil PO1; PO2; PO3; PO4; or | Parfum | 0.5 |
| PO5 | | |
| Hydrolite ® 5 green | Pentylene Glycol | 3.0 |
| Sym Triol | Caprylyl Glycol, 1,2-Hexanediol, | 0.5 |
| | Methylbenzyl alcohol | |
| 1,2-Nonanediol | 1,2-Nonanediol | 0.2 |
| 1,2-Heptanediol/2,3-Heptanediol | 1,2-Heptanediol, 2,3-Heptanediol | 0.5 |
| (w/w % 95:5) | | |
| Frescolat ® ML | Mentyl Lactate | 1.0 |
| Frescolat MGA | Menthone Glycerin Acetal | 0.4 |
| Actipone ® Camu Camu GW | Aqua, Glycerin, Myrciaria dubia | 0.5 |
| | fruit juice | |
| SymRelief ® S | Bisabolol, Hydroxymethoxyphenyl | 0.05 |
| | Decanone | |
| SymRepair ® 100 | Hexyldecanol, Bisabolol, | 1.0 |
| | Cetylhydroxyproline Palmitamide, | |
| | Stearic Acid, Brassica Campestris | |
| | (Rapeseed), Sterols | |

TABLE 73
Refresh your Breath Mouthspray

| Raw material | INCI | Amount |
|---|---|---|
| Aqua/Water | Aqua | ad 100 |
| Sorbitol Liq. EP 70% | Sorbitol | 10.0 |
| Sodium Saccharin | Sodium Saccharin | 0.1 |
| Cremophor ® CO 455 | PEG-40 Hydrogenated castor oil, | 5.0 |
| | Propylene Glycol | |
| Flavour | Aroma | 0.8 |
| Frescolat ® ML Crst | Menthyl Lactate | 2.0 |
| Propylene Glycol | Propylene Glycol | 10.0 |
| SymOcide ® C | o-Cymen-5-ol | 0.1 |
| SymSave ® H | Hydroxyacetophenone | 0.25 |
| SymDiol ® 68 | 1,2-Hexanediol, Caprylyl Glyol | 0.5 |
| 1,2-Nonanediol | 1,2-Nonanediol | 0.1 |
| 1,2-Heptanediol/ | 1,2-Heptanediol, 2,3-Heptanediol | 0.6 |
| 2,3-Heptanediol | | |
| (w/w % 95:5) | | |
| Extrapone ® Peppermint | Aqua, Propylene Glycol, Mentha | 2.0 |
| | piperita leaf extract | |
| Colour | | 0.2 |

TABLE 74
Sheet mask—Ultra Calming

| Raw material | INCI | Amount |
|---|---|---|
| Aqua/Water | Aqua | ad 100 |
| SymSave ® H | Hydroxyacetophenone | 0.5 |
| EDTA NA2 | Disodium EDTA | 0.1 |
| Glycerin | Glycerin | 6.0 |
| Hydrolite ® 5 green | Pentylene Glycol | 3.0 |
| Sepimax ™ ZEN | Polyacrylate Crosspolymer-6 | 0.5 |
| Jojoba Oil | Simmondsia chinensis seed oil | 4.0 |
| Caprylic/Capric Triglyceride | Caprylic/Capric Triglyceride | 8.0 |
| Dracorin ® GOC | Glyceryl Oleate citrate, | 2.0 |
| | Caprylic/Capric Triglyceride | |
| Frescolat ® ML Cryst | Menthyl Lactate | 0.2 |
| N-Ethyl-p-menthane-3-carboxamide | Ethyl Menthane Carboxamide | 0.6 |
| (Cooling substance WS-3) | | |
| 2,3-Nonanediol | 2,3-Nonanediol | 0.1 |
| 1,2-Heptanediol/2,3-Heptanediol | 1,2-Heptanediol, 2,3-Heptanediol | 0.6 |
| (w/w % 95:5) | | |
| SymDecanox ™ HA | Caprylic/Capric Triglyceride, | 1.0 |
| | Hydroxymethoxyphenyl Decanone | |
| SymSitive ® 1609 | Pentylene Glycol, 4-t- | 1.2 |
| | Butylcyclohexanol | |
| Simulgel NS | Hydroxyethyl acrylate/Sodium | 0.7 |
| | acryloyldimethyl taurate | |
| | copolymer, Sqalane, Poylsorbate | |
| | 60 | |
| SymCalmin ® green | Pentylene Glycol, Butylene | 2.0 |
| | Glycol, Hydroxyphenyl | |
| | Propamidobenzoic acid | |
| SymRelief ® 100 | Bisabolol, Zingiber officinale Root | 0.05 |
| | extract | |
| Extraone ® Watermin P | Aqua, Propylene Glycol, Glucose, | 1.0 |
| | Mentha Aquatica Leaf extract | |
| Perfume oil PO1; PO2; PO3; PO4; or PO5 | Parfum | 0.15 |
| Colour | | 0.4 |

TABLE 75
Watermelon Face Mask & Scrub

| Raw material | INCI | Amount |
|---|---|---|
| Aqua/Water | Aqua | ad 100 |
| SymSave ® H | Hydroxyacetophenone | 0.5 |
| Hydroviton ® PLUS 2290 | Aqua, Pentylene Glycol, Glycerin, | 2.0 |
| | Fructose, Urea, Citric Acid, | |
| | Sodium Hydroxide, Maltose, | |
| | Sodium PCA, Sodium Chloride, | |
| | Sodium Lactate, Trehalose, | |
| | Allantoin, Sodium Hyaluronate, | |
| | Glucose | |
| Edeta BD | Disodium EDTA | 0.1 |
| Extrapone ® Watermelon GW | Aqua, Glycerin, Citrullus Lanatus | 1.0 |
| | (Watermelon) Fruit Juice | |
| Colour | | 0.3 |
| SymSol ® PF-3 | Aqua, Pentylene Glycol, Sodium | 3.0 |
| | Lauryl Sulfoacetate, Sodium | |
| | Oleoyl Sarcosinate, Sodium | |
| | Chloride, Soium Oleate | |
| SymRelief 100 | Bisabolol, Zingiber officinale root | 0.1 |
| | extract | |
| Perfume oil PO1; PO2; PO3; PO4; or PO5 | Parfum | 0.2 |
| Frescolt ® Plus | Menthol, Menthyl Lactate | 0.2 |
| 1,2-Nonanediol | 1,2-Nonanediol | 0.5 |
| 1,2-Heptanediol/2,3-Heptanediol | 1,2-Heptanediol, 2,3-Heptanediol | 1.0 |
| (w/w % 95:5) | | |
| Hydrolite ® 5 green | Pentylene Glycol | 3.0 |
| Aqua Keep 10SH-NFC | Sodium Acrylates Crosspolymer-2 | 1.5 |
| Keltrol ® CG-T | Xanthan Gum | 0.3 |
| Tinogard Q | Tris(Tetramethylhydroxy- | 0.05 |
| | piperidinol) Citrate | |
| Kiwi Exfoliator 2000 | Actinidia chinensis (Kiwi) Seed | 1.0 |

TABLE 76
Illuminating Snow MASK with Glacier Water

| Raw material | INCI | Amount |
|---|---|---|
| Aqua/Water | Aqua | ad 100 |
| SymSave ® H | Hydroxyacetophenone | 0.5 |
| EDTA NA2 | Disodium EDTA | 0.1 |
| Emulsiphos ® | Potassium Cetyl Phosphate, | 4.0 |
| | Hydrogenated Palm Glycerides | |
| Dragosine ® | Carnosine | 0.2 |
| Lanette ® O | Cetearyl Alcohol | 2.0 |
| Lanette ® 22 | Beheny Alcohol | 1.0 |
| Nat Rice Bran Wax | Oryza Sativa Bran Cera | 0.5 |
| White Beeswax BP | Cera Alba | 1.5 |
| Murumuru Butter | Astrocaryum Murumuru Seed | 2.0 |
| | Butter, Helianthus Annuus Seed | |
| | oil, Tocopherol | |
| PCL-Solid | Stearyl Heptanoate, Stearyl | 2.0 |
| | Caprylate | |
| PCL-Liquid ® | Cetearyl Ethylhexanoate, | 4.0 |
| | Isopropyl Myristate | |
| Sweet Almond Oil (Organic) | Prunus amygdalus dulcis (Sweet | 3.0 |
| | almond) oil | |
| SymDecanox ™ HA | Caprylic/Capric Triglyceride, | 1.0 |
| | Hydroxymethoxyphenyl Decanone | |
| SymBright ® 2036 | Sclareolide | 0.1 |
| Frescolat ® X-Cool | Menthyl ethylamido oxalate | 0.3 |
| Aqua Keep 10SH-NFC | Sodium acrylates Crosspolymer-2 | 2.0 |
| Covacryl MV60 | Sodium Polyacrylate | 1.0 |
| Hydrolite ® 5 green | Pentylene Glycol | 3.0 |
| 1,2-Heptanediol/2,3-Heptanediol | 1,2-Heptanediol, 2,3-Heptanediol | 1.0 |
| (w/w % 95:5) | | |
| 2,3-Nonanediol | 2,3-Nonanediol | 0.05 |
| Extrapone ® Glacier Water GW | Glycerin, Aqua | 1.0 |
| Tapioca Pure | Tapioaca Starch | 1.5 |
| SunShine Glitter Gold C81-3410 | Fluorphlogopite (and) Titanium | 0.4 |
| | dioxide | |
| Perfume oil PO1; PO2; PO3; PO4; or PO5 | Parfum | 0.3 |

TABLE 77
Syndet antimicrobial soap bar

| Ingredients | INCI | Amount |
|---|---|---|
| Zetesap 813 A | Disodium Lauryl Sulfosuccinate, | ad 100 |
| | Sodium Lauryl Sulfate, Corn | |
| | Starch, Cetearyl Alcohol, | |
| | Paraffin, Titanium Dioxide | |
| Amphotensid GB 2009 | Disodium Cocoamphodiacetate | 6.0 |
| Allantoin | Allantoin | 1.0 |
| Perfume oil PO1; PO2; | Fragrance | 1.0 |
| PO3; PO4 or PO5 | | |
| SymOcide C | o-Cymen-5-ol | 0.1 |
| 1,2-Heptanediol/ | 1,2-Heptanediol/2,3-Heptanediol | 5.0 |
| 2,3-Heptanediol | | |
| blend (95:5 w/w %) | blend (95:5 w/w %) | |

TABLE 78
Syndet soap bar

| Ingredients | INCI | Amount |
|---|---|---|
| Fenopon AC-78 | Sodium Cocoyl Isethionate | 20.0 |
| Natriumlaurylsulfoacetate | Sodium Lauryl Sulfoacetate | 16.0 |
| Paraffin | Paraffin | 19.0 |
| Wax, microcrystalline | Microcrystalline Wax | 1.0 |
| Corn Starch | Corn Starch | 8.0 |
| Coconut acid | Coconut acid | 2.0 |
| Lauric acid diethanol amide | Lauramide DEA | 2.0 |
| Dextrin | Dextrin | 21.0 |
| Lactic acid, 88% | Lactic Acid | 1.0 |
| Sym Guard CD | 3-Phenylpropanol, o-Cymen- | 0.3 |
| | 5-ol, Decylene glycol | |
| Thymol | Thymol | 0.05 |
| Water | Water | ad 100 |
| Perfume oil PO1; PO2; | Fragrance | 1.0 |
| PO3; PO4 or PO5 | | |
| 1,2-Heptanediol/ | 1,2-Heptanediol/2,3- | 3.0 |
| 2,3-Heptanediol | Heptanediol blend | |
| blend (95:5 w/w %) | (95:5 w/w %) | |

TABLE 79
Antimicrobial toilet soap bar

| Ingredients | Amount |
|---|---|
| Sodium soap from tallow | 60.0 |
| Sodium soap from palm oil | 27.0 |
| Glycerol | 2.0 |
| Sodium Chloride | 0.5 |
| 1-Hydroxyethane-1,1-diphosphonic acid, tetrasodium salt | 0.3 |
| Alpha-Tocopherol | 0.1 |
| Pigment Yellow 1 | 0.02 |
| Water | ad 100 |
| Perfume oil PO1; PO2; PO3; PO4 or PO5 | 3.0 |
| Farnesol | 0.2 |
| 2,3-Heptanediol | 1.0 |
| 2,3-Octanediol | 1.0 |

TABLE 80
Ice Crystal Soap

| Raw material | INCI | Amount |
|---|---|---|
| Crystal SLEA/SLS Free | Aqua, Glycerin, Sorbitol, Sodium | ad 100 |
| | stearate, Sodium Laurate, | |
| | propylene Glycol, Sodium Oleate, | |
| | Sodium Myristate, Sodium | |
| | chloride, Glyceryl Laurate, | |
| | Cocaidopropyl Betaine, Cocos | |
| | Nucifera oil, Sodium Thiosulfate, | |
| | Sodium Citrate, Citric Acid, | |
| | Tetrasodium Iminodisuccinate, | |
| | Tetrasodium Etidronate | |
| Frescolat MGA | Menthone Glycerin Acetale | 2.0 |
| 1,2-Heptanediol | 1,2-Heptanediol | 0.5 |
| 2,3-Nonanediol | 2,3-Nonanediol | 0.05 |
| Menthol | Menthol | 0.05 |
| Extrapone Glacie | Glycerin, Aqua | 1.0 |
| Water GW | | |
| Colour I | | 5.0 |
| Colour II | | 3.5 |

TABLE 81
Air freshener (A/F spray water based)

| No | Ingredients | Amount |
|---|---|---|
| 1 | Fragrance | 3.8 |
| 2 | Menthol | 0.2 |
| 3 | Ethanol | 8 |
| 4 | PEG-40 Hydrogenated Castor Oil | 5 |
| 5 | 1,2-heptanediol | 1 |
| 6 | Aqua | add to 100 |

Colorless liquid, pH neutral

1. A homecare product composition or cosmetic or pharmaceutical composition comprising:
(a1) 1,2-nonanediol;
(b 1) at least one cooling agent;
(c1) optionally at least one antimicrobial compound; and
(d1) optionally at least one active substance for a cosmetic or pharmaceutical composition or a homecare product composition and/or additive;
or
(a2) 2,3-nonanediol;
(b2) at least one cooling agent;
(c2) optionally at least one antimicrobial compound; and
(d2) optionally at least one active substance for a cosmetic or pharmaceutical composition or a homecare product composition and/or additive;
or
(a3) a mixture comprising 1,2-nonanediol and 2,3-nonanediol;
(b3) at least one cooling agent;
(c3) optionally at least one antimicrobial compound; and
(d3) optionally at least one active substance for a cosmetic or pharmaceutical composition or a homecare product composition and/or additive.

2. A homecare product composition or cosmetic or pharmaceutical composition comprising:
(a) at least one linear alkanediol having a carbon chain of 5 to 14 carbon atoms or a mixture of at least one first linear alkanediol having a carbon chain of 5 to 14 carbon atoms with one or more second linear alkanediol(s) having a carbon chain of 5 to 14 carbon atoms which is different to the first linear alkanediol;
(a) at least one cooling agent;
(b) optionally at least one antimicrobial compound; and
(c) optionally at least one active substance for a cosmetic or pharmaceutical composition or a homecare product composition and/or additive.

3. The homecare product composition or cosmetic or pharmaceutical composition according to claim 2, wherein the at least one linear alkanediol or the at least one first linear alkanediol is selected from the group consisting of pentanediol, hexanediol, heptanediol, octanediol, nonanediol, decanediol, undecanediol, dodecanediol, and tridecanediol.

4. (canceled)

5. The homecare product composition or cosmetic or pharmaceutical composition according to claim 2, wherein the one or more second linear alkanediol(s) is selected from the group consisting of pentanediol, hexanediol, heptanediol, octanediol, nonanediol, decanediol, undecanediol, dodecanediol, and tridecanediol.

6. The homecare product composition or cosmetic or pharmaceutical composition according to claim 2, wherein the at least one linear alkanediol, the at least one first linear alkanediol and/or the one or more second linear alkanediol(s) is a (x,x+1)-constitutional isomer, wherein x stands for the number of the carbon atom in the alkanediol chain to which the OH groups of the alkanediol are chemically bonded.

7. The homecare product composition or cosmetic or pharmaceutical composition according to claim 2, wherein the at least one linear alkanediol or the at least one first linear alkanediol, is selected from the group consisting of:
1,2-pentanediol, 2,3-pentanediol,
1,2-hexanediol, 2,3-hexanediol,
1,2-heptanediol, 2,3-heptanediol,
1,2-octanediol, 2,3-octanediol,
1,2-nonanediol, 2,3-nonanediol,
1,2-decanediol, 2,3-decanediol,
1,2-undecanediol, 2,3-undecanediol,
1,2-dodecanediol, 2,3-dodecanediol,
1,2-tridecanediol, 2,3-tridecanediol, and mixtures thereof;
and/or
wherein the one or more second linear alkanediol(s) is selected from the group consisting of:
1,2-pentanediol, 2,3-pentanediol,
1,2-hexanediol, 2,3-hexanediol,
1,2-heptanediol, 2,3-heptanediol,
1,2-octanediol, 2,3-octanediol,
1,2-nonanediol, 2,3-nonanediol,
1,2-decanediol, 2,3-decanediol,
1,2-undecanediol, 2,3-undecanediol,
1,2-dodecanediol, 2,3-dodecanediol,
1,2-tridecanediol, 2,3-tridecanediol, and mixtures thereof.

8. The homecare product composition or cosmetic or pharmaceutical composition according to claim 7, wherein the mixture of at least one first linear alkanediol with one or more second linear alkanediol(s) is selected from the group consisting of
a mixture comprising 1,2-pentanediol and 2,3-pentanediol;
a mixture comprising 1,2-hexanediol and 2,3-hexanediol;
a mixture comprising 1,2-heptanediol and 2,3-heptanediol;
a mixture comprising 1,2-heptanediol and 2,3-octanediol;
a mixture comprising 1,2-heptanediol and 1,2-nonanediol;
a mixture comprising 1,2-heptanediol and 2,3-nonanediol;
a mixture comprising 1,2-heptanediol and 1,2-undecanediol;
a mixture comprising 1,2-heptanediol and 2,3-undecanediol;
a mixture comprising 2,3-heptanediol and 1,2-nonanediol;
a mixture comprising 2,3-heptanediol and 2,3-nonanediol;
a mixture comprising 2,3-heptanediol and 1,2-undecanediol;
a mixture comprising 2,3-heptanediol and 2,3-undecanediol;
a mixture comprising 1,2-octanediol and 2,3-octanediol;
a mixture comprising 1,2-nonanediol and 2,3-nonanediol;
a mixture comprising 1,2-nonanediol and 1,2-undecanediol;
a mixture comprising 1,2-nonanediol and 2,3-undecanediol;
a mixture comprising 2,3-nonanediol and 1,2-undecanediol;
a mixture comprising 2,3-nonanediol and 2,3-undecanediol;
a mixture comprising 1,2-decanediol and 2,3-decanediol;
a mixture comprising 1,2-undecanediol and 2,3-undecanediol;
a mixture comprising 1,2-dodecanediol and 2,3-dodecanediol;
a mixture comprising 1,2-tridecanediol and 2,3-tridecanediol;
a mixture comprising 1,2-hexanediol and 2,3-octanediol;
a mixture comprising 1,2-octanediol and 2,3-hexanediol;
a mixture comprising 1,2-octanediol and 2,3-heptanediol;
a mixture comprising 1,2-pentanediol and 2,3-hexanediol;
a mixture comprising 1,2-pentanediol and 1,2-heptanediol;
a mixture comprising 1,2-pentanediol and 2,3-heptanediol;
a mixture comprising 1,2-pentanediol and 2,3-octanediol;
a mixture comprising 1,2-pentanediol and 1,2-nonanediol;
a mixture comprising 1,2-pentanediol and 2,3-nonanediol;
a mixture comprising 1,2-heptanediol and 1,2-octanediol.

9-11. (canceled)

12. The homecare product composition or cosmetic or pharmaceutical composition according to claim 7, wherein the at least one linear alkanediol is selected from the group consisting of 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol and mixtures thereof.

13. The homecare product composition or cosmetic or pharmaceutical composition according to claim 2,
wherein the mixture of at least one first linear alkanediol and one or more second linear alkanediol(s) comprises the first linear alkanediol and the second alkanediol in a ratio in a range of 50:50 to 99.9:0.1.

14. The homecare product composition or cosmetic or pharmaceutical composition according to claim 2, comprising a mixture of:
1,2-heptanediol and 1,2-octanediol; or
1,2-heptanediol and 2,3-heptanediol; or
1,2-octanediol and 2,3-octanediol; or
1,2-nonanediol and 2,3-nonanediol.

15. The homecare product composition or cosmetic or pharmaceutical composition according to claim 2, wherein the at least one cooling agent is selected from the group consisting of menthol, menthol derivatives, cyclohexanol derivatives, cyclic secondary and tertiary alcohols, alkylmethanols, trialkylphosphine oxides, cyclic and acyclic sulfoxides, cyclic and acyclic sulfones, cyclic amides, acyclic amides, menthol-derived amides and carboxamides.

16. The homecare product composition or cosmetic or pharmaceutical composition according to claim 2, wherein the at least one cooling agent is selected from the group consisting of menthol, menthol derivatives and menthol-derived amides.

17. The homecare product composition or cosmetic or pharmaceutical composition according to claim 2, wherein the at least one antimicrobial compound is selected from the group consisting of ethylhexylglycerin, triethyl citrate, polyglyceryl-3 caprylate, farnesol, and 2-methyl 5-cyclohexylpentanol.

18. The homecare product composition or cosmetic or pharmaceutical composition according to claim 2, wherein the at least one linear alkanediol, the at least one first linear alkanediol and the one or more second linear alkanediol(s) are selected from the group consisting of 1,2-heptanediol, 2,3-heptanediol, 1,2-nonanediol, 2,3-nonanediol, 1,2-undecanediol and 2,3-undecanediol;
wherein the at least one cooling agent is selected from the group consisting of menthol, D-menthol, L-menthol, (−)-menthyl lactate, 3-((−)-menthoxy)propane-1,2-diol, (−)-menthone 1,2-glycerol ketal, N-ethyl-p-menthane-3-carboxamide and Menthyl ethylamido oxalat; and
wherein the composition comprises an antimicrobial compound which is selected from the group consisting of ethylhexylglycerin, triethyl citrate, polyglyceryl-3 caprylate, farnesol and 2-methyl 5-cyclohexylpentanol.

19. The homecare product composition or cosmetic or pharmaceutical composition according to claim 2, wherein the at least one active substance for a cosmetic or pharmaceutical composition or a homecare product composition and/or additive is selected from the group consisting of agents against ageing of the skin, antioxidants, chelating agents, emulsifiers, preservatives, green and synthetic polymers, rheology additives, oils, fragrances or perfume oils, polyols and mixtures of two or more of the afore-mentioned substances.

20. The homecare product composition or cosmetic or pharmaceutical composition according to claim 1, comprising the 1,2-nonanediol and/or the 2,3-nonanediol or the at least one linear alkanediol or the mixture of at least one first linear alkanediol with one or more second linear alkanediol(s) in an amount of 0.001 to 15.0% by weight based on the total weight of the composition or a final homecare product or cosmetic or pharmaceutical preparation.

21. The homecare product composition or cosmetic or pharmaceutical composition according to claim 2, comprising the at least one cooling agent in an amount of 0.0001 to 10.0% by weight based on the total weight of the composition.

22. The homecare product composition or cosmetic or pharmaceutical composition according to claim 1, comprising
the 2,3-nonanediol or the 2,3-nonanediol of the mixture comprising 1,2-nonadiol and 2,3-nonanediol in an amount of 0.001 to 15.0% by weight, based on the total weight of the composition or a final cosmetic or pharmaceutical preparation or homecare product.

23. A method of reducing malodour, inhibiting malodour development, controlling body odour, or masking insect alluring odours comprising administering a homecare product composition or cosmetic or pharmaceutical composition according to claim 2.

24. A cosmetic product comprising a cosmetic composition according to claim 2, wherein the product is a deodorant and/or antiperspirant, an aerosol-based deo spray, deo pump spray, deo stick, deo roll on, deo cream, deo wipes, deo crystals, pickering emulsions, hydrodispersion gels, skin balms, shampoo, shower gel, foam bath, micellar water, facial cleansing solutions, cleansing wipes, intimate spray, intimate cream, intimate wash lotion, intimate wipes, aerosol-based foot spray, foot pump spray, foot bath, foot balm, soap, liquid washing, shower and bath preparation, bath product, bath capsule, bath oil, bath tablet, bath salt, bath soap, effervescent preparation, mouth wash concentrate, mouth wash ready to use, repellent, anti-insect application, human-smell masking application, perfume composition or tooth paste.

25. (canceled)
